(19) United States
(12) Patent Application Publication
(10) Pub. No.: US 2021/0239678 A1
(43) Pub. Date: Aug. 5, 2021

(54) LEUKOCYTE RECRUITMENT IN INFECTIOUS DISEASE

(71) Applicant: Stickycell Pty Ltd, Aspley (AU)

(72) Inventors: Qiang Cheng, Aspley (AU); Anqi Li, Heathmont (AU); Karina Islas Rios, Notting Hill (AU); Erick Vargas-Ordaz, Notting Hill (AU)

(21) Appl. No.: 17/048,656

(22) PCT Filed: Apr. 18, 2019
(86) PCT No.: PCT/AU2019/050354

§ 371 Date: Oct. 19, 2020

(30) Foreign Application Priority Data
Apr. 19, 2018 (AU) 2018901305

(51) Int. Cl. G01N 33/49

(52) U.S. Cl. CPC G01N 33/49; G01N 2496/00; G01N 2800/26; G01N 2333/5421; G01N 2333/47; G01N 2333/70564; G01N 2333/70525; G01N 2800/52

(57) ABSTRACT

The present disclosure relates to assays, including but not limited to, leukocyte adhesive function assays (LAFA), devices and/or methods of using such assays. The present disclosure also relates to the uses of the disclosed embodiment in diagnostic, analytic and/or prognostic applications, particularly for diagnostic, analytic and/or prognostic applications in relation to diseases associated with abnormal host immune responses.

### TECHNICAL FIELD

The present disclosure relates to assays, including but not limited to, leukocyte adhesive function assays (LAFA), devices and/or methods of using such assays. The present disclosure also relates to the uses of the disclosed embodiment in diagnostic, analytic and/or prognostic applications, particularly for diagnostic, analytic and/or prognostic applications in relation to diseases associated with abnormal host immune responses.

### BACKGROUND

Systemic Inflammatory Response Syndrome (SIRS) is a complex inflammatory whole-body response to non-infectious or infectious foreign insults. Non-infectious causes include major surgery, trauma, burns and severe tissue injury; whereas infectious causes, such as bacterial, fungal or viral, clinically define the sepsis syndromes. In clinic, infectious and non-infectious SIRS share an almost identical pathophysiology.

Due to different aetiology, the treatments for non-infectious and infectious causes of systemic inflammation differ dramatically. Antibiotic therapy and source control are the first line of treatment for sepsis, whilst antibiotic therapy is not indicated for non-infectious causes. For most of the patients admitted to ICU with systemic inflammation, however, it is difficult to rapidly and accurately determine disease pathogenesis, due to a great similarity between these two diseases. The current practice is that patients will be given antibiotics once potential infection is suspected, frequently leading to overuse of antibiotics for patients with non-infectious causes. Antibiotics are continued until the treating clinician is satisfied that the cause of the systemic inflammation is not sepsis. This inappropriate use of antibiotics in patients with non-infectious causes may result in the emergence of resistant pathogens, as well as other avoidable side effects. Thus, in clinic, it is useful to accurately and rapidly distinguish sepsis from non-infectious SIRS, so that more targeted therapies may be applied early to improve survival rate and avoid treatment side effects.

To differentiate sepsis from non-infectious SIRS, most work focused on the determination and characterisation of potential pathogens in patient blood and/or tissues. Currently, blood culture is the standard for the identification of infectious pathogen in SIRS patients. However, the result turn-around for blood culture usually requires 12-72 hours, whereas the guidelines recommend antibiotic administration within 1 hour of infection suspicion. More recently, other molecular tests have become commercially available for the detection of a number of micro-organisms, including Prove-it™ assay, Verigene®, FilmArray® and PNA-FISH. However, the lack of rapidity, low sensitivity and specificity have been preventing the widespread use of these techniques in clinical setting. Despite being a useful indication for septic state, it was found that up to 50% of the sepsis patients had negative blood culture results and the culture positivity is not mandatory for the diagnosis of sepsis. Thus, the disease conditions do not only present the infection itself, but the reaction of the host immune system in response to the infection. There has been work done on the identification of biomarkers in the host immune system, aiming to determine the status of host immune response and assist the diagnosis of sepsis. These markers include precalcitonin (PCT), C-reactive protein (CRP), triggering receptor expression on myeloid cells 1 (TREM-1) and decoy receptor 3 (DCR3). The lack of rapidity and specificity when assaying for these markers remains a significant drawback of these techniques.

Thus, there remains a need for methods, systems and/or to devices to distinguish between sepsis and non-infectious inflammatory conditions, and to predict a subject's potential response to treatment for systemic inflammation having an infectious cause versus a non-infectious cause.

### SUMMARY

In some embodiments, the present disclosure provides a method to discriminate between an infectious and non-infectious inflammatory immune response in a subject, the method comprising:

subjecting a blood sample from the subject to at least one leukocyte function assay (LAFA), wherein the LAFA assesses leukocyte recruitment, adhesion and/or migration to at least one endothelial cell molecule; and based at least in part on one or more results of the at least one LAFA, determine whether the subject has an infectious inflammatory immune response or a non-infectious inflammatory immune response.

In one embodiment, the at least one LAFA quantitatively and/or semi-quantitatively assesses leukocyte recruitment, adhesion and/or migration.

In one embodiment, the method comprises obtaining a blood sample from the subject.

In one embodiment, the at least one endothelial cell molecule is selected from VCAM-1, MadCAM-1, IL-8, SDF-1α, E-Selectin, P-Selectin and ICAM-1.

In an embodiment, the at least one endothelial cell molecule comprises two or more of VCAM-1, MadCAM-1, IL-8, SDF-1α, E-Selectin, P-Selectin and ICAM-1.

In one embodiment, the at least one LAFA measures one or more of the following parameters: a quantification of rolling leukocyte cells detected, a quantification of adhesion leukocyte cells detected, a quantification of crawling cells detected, an average speed of individual leukocyte cells detected, an average straightness of individual leukocyte cells detected, an average displacement of individual leukocyte cells detected and an average dwell time of individual cells detected.

In one embodiment, the results of the at least one LAFA from the blood sample from the subject is used as a reference level for generating one or more parameters that are used for generating one or more indexes.

In one embodiment, the results of the at least one LAFA from at least one healthy blood sample is used as a reference level for generating one or more parameters that are used of generating one or more indexes.

In one embodiment, an activation potential ratio of the subject's blood is generated based on the results of at least one LAFA from the blood of the subject divided by the results of at least one LAFA from a Mn2+ treated blood sample of the subject.

In one embodiment, the method further comprises detecting one or more leukocyte cell surface markers.

In one embodiment, the one or more leukocyte cell markers are selected from CD4, CD8, CD14, CD15, CD16, CD19 and CD25.

In one embodiment, the subject has, or is suspected of having, systemic inflammatory response syndrome (SIRS).

In one embodiment, the method comprises comparing leukocyte recruitment, adhesion, and/or migration to a reference level of leukocyte recruitment, adhesion, and/or migration.

In one embodiment, the reference level of leukocyte recruitment, adhesion, and/or migration is derived from an established data set.

In one embodiment, the established data set comprises measurements of leukocyte recruitment, adhesion, and/or migration for a population of subjects known to have an infectious inflammatory immune response and/or a population of subjects known to have a non-infectious inflammatory immune response.

In one embodiment, the population of subjects known to have an infectious inflammatory immune response are known to have sepsis.

In one embodiment, the population of subjects known to have a non-infectious inflammatory immune response are known to have SIRS.

In one embodiment, a LAFA result comprises:

i) a higher or lower level of recruited and/or adhesive leukocytes;

ii) a higher or lower percentage of recruited and/or adhesive neutrophils; and/or

iii) a higher or lower level of recruited and/or adhesive monocytes,

as compared to the reference level is indicative of sepsis, wherein the reference level is derived from a population of subjects known to have non-infectious SIRS.

In one embodiment, the method comprises determining that the subject has an infectious inflammatory immune response and administering an antimicrobial or antiviral composition to the subject.

In another embodiment, the method comprises determining that the subject has a non-infectious inflammatory immune response and administering an anti-inflammatory composition to the subject.

In one embodiment, the method comprises determining that the subject has a non-infectious inflammatory response and administering to the subject a drug capable of altering leukocyte recruitment, adhesion and/or migration.

In one embodiment, for example, the drug may be an antibody that interferes with the binding of a leukocyte adhesion molecule to an endothelial cell molecule.

In certain embodiments, the drug may be an antibody that interferes with the binding between α4 integrin and its endothelial molecule. The drug may be an anti-human α4 integrin antibody. In certain embodiments, the drug is Natalizumab.

In certain embodiments, the drug may be an antibody that interferes with the binding between α4β7 integrin and MAdCAM-1. The drug may be, for example, Vedolizumab.

In certain embodiments the drug may be an antibody that interferes with the binding between CD11α (αL) and ICAM-1. The drug may be, for example, Efalizumab or Odulimomab.

In certain embodiments, the drug may be an antibody that interferes with the binding between CD11b (αM) and ICAM-1. The drug may be, for example, UK279, or UK276.

In certain embodiments, the drug may be an antibody that interferes with the binding between β2 integrin and its endothelial molecule. The drug may be, for example, Erlizumab or Roverlizumab.

In certain embodiments the drug may be an antibody that interferes with the binding between β7 integrin its endothelial molecule. The drug may be, for example, Etrolizumab.

In some embodiments, there is provided a method of treating an infectious inflammatory immune response in a subject, the method comprising performing the method as described herein and determining that the subject has an inflammatory immune response, and treating the subject for the inflammatory immune response.

In one embodiment, the subject has sepsis.

In one embodiment, treating the subject for sepsis comprises treating the patient with one or more of an antibiotic, vasopressor and corticosteroid.

In some embodiments, there is provided a method to assess a subject's response, or potential response, to a drug suitable for treating an infectious disease, the method comprising:

subjecting a blood sample from the subject to at least one leukocyte function assay (LAFA), wherein the LAFA assesses leukocyte recruitment, adhesion and/or migration to at least one endothelial cell molecule; and

based at least in part on one or more results of the at least one LAFA, assess a patient's response, or potential response, to the drug for treating the infectious disease.

In some embodiments, there is provided a method of detecting a subset of leukocytes in a subject having an inflammatory immune response, the method comprising subjecting a blood sample from the subject to at least one leukocyte function assay (LAFA), wherein the LAFA assesses leukocyte recruitment, adhesion and/or migration to at least one endothelial cell molecule;

detecting one or more leukocyte cell surface markers, and

based at least in part on one or more results of the at least one LAFA and detection of one or more leukocyte cell surface markers, determining a subset of leukocytes associated with the inflammatory immune response.

In one embodiment, the method comprises detecting multiple leukocyte cell surface markers and/or detecting multiple subsets of leukocytes.

In one embodiment, the subject has an inflammatory condition or infectious disease.

In one embodiment, the subject has, or is suspected of having, SIRS.

In one embodiment, the subject has sepsis.

In some embodiments, there is provided a method for determining a cause of inflammation in a subject, the method comprising subjecting a blood sample from the subject to at least one leukocyte function assay (LAFA), wherein the LAFA assesses leukocyte recruitment, adhesion and/or migration to at least one endothelial cell molecule; and

based at least in part on one or more results of the at least one LAFA, determine the cause of inflammation in the subject.

In one embodiment, the method further comprises detecting one or more leukocyte cell surface markers.

In one embodiment, the one or more leukocyte cell markers are selected from CD4, CD8, CD14, CD15, CD16, CD19 and CD25.

In one embodiment, the method comprises detecting multiple leukocyte cell surface markers and/or detecting multiple subsets of leukocytes.

In one embodiment, the cause of inflammation in the subject is determined to be an infectious cause of inflammation.

In one embodiment, the infectious cause of inflammation is a bacterial, viral or parasitic infection.

In one particular embodiment, the bacterial infection is selected from an infection caused by one or more of an enteric bacterium, Serratia sp., Pseudomonas sp., E. coli, and Staphylococcus aureus.

In one embodiment, the cause of inflammation in the subject is determined to be a non-infectious cause of inflammation.

In one embodiment, the non-infectious cause of inflammation is selected from myocardial infarction, asthma, haemorrhage, aneurysm and/or pneumonitis.

In some embodiments, there is provided a system for performing the at least one LAFA based on the method as described herein

In some embodiments, there is provided a device for performing the at least one LAFA based on the methods as described herein.

In some embodiments, there is provided a method to discriminate between an infectious and non-infectious inflammatory immune response in a subject, the method comprising:

subjecting a blood sample from the subject to at least one leukocyte function assay (LAFA), wherein the LAFA captures video data of leukocyte recruitment, adhesion and/or migration to at least one endothelial cell molecule; and

applying machine learning to the video data to determine whether the subject has an infectious inflammatory immune response or a non-infectious inflammatory immune response.

In one embodiment, the video data comprises multiple images and applying machine learning to the video data comprises:

combining the multiple images into a single image; and a

applying machine learning to the single image.

In one embodiment, combining the multiple images comprises performing maximum intensity projection to combine the multiple images into the single image.

In one embodiment, applying machine learning comprises applying a convolutional neural network to the single image.

In one embodiment, applying the convolutional neural network to the single image comprises training the convolutional neural network using a single training image for each of multiple training samples with infectious and non-infectious inflammatory immune response and applying the trained convolutional neural network to the single image for the subject under examination.

In one embodiment, the method further comprises:

performing cell tracking to determine cell tracking parameter values; and

applying machine learning to the cell tracking parameter values.

In one embodiment, the method comprises applying machine learning to the cell tracking parameter values comprises applying a random forest to the cell tracking parameters.

In one embodiment, the cell tracking parameters are represented by nodes of trees in the random forest.

In on embodiment, applying the random forest to the single image comprises training the random forest using a single training image for each of multiple training samples with infectious and non-infectious inflammatory immune response and applying the trained random forest to the single image for the subject under examination.

In some embodiments, there is provided a method to determine the cause of inflammation in a subject, the method comprising:

subjecting a blood sample from the subject to at least one leukocyte function assay (LAFA), wherein the LAFA captures video data of leukocyte recruitment, adhesion and/or migration to at least one endothelial cell molecule; and applying machine learning to the video data to determine the cause of inflammation in the subject.

In some embodiments, there is provided a method for pre-symptomatic detection of infection in a subject, the method comprising:

subjecting a blood sample from the subject to at least one leukocyte function assay (LAFA), wherein the LAFA assesses leukocyte recruitment, adhesion and/or migration to at least one endothelial cell molecule; and

based at least in part on one or more results of the at least one LAFA, determine whether the subject has an infection.

In some embodiments, the infection is a viral infection. For example, in one embodiment the infection may be an influenza infection.

In some embodiments, there is provided a method for the pre-symptomatic detection of SIRS, the method comprising:

subjecting a blood sample from the subject to at least one leukocyte function assay (LAFA), wherein the LAFA assesses leukocyte recruitment, adhesion and/or migration to at least one endothelial cell molecule; and based at least in part on one or more results of the at least one LAFA, determine whether the subject has SIRS.

In some embodiments, the SIRS is infectious SIRS.

In some embodiments, the SIRS is non-infectious SIRS.

In some embodiments, a reduction in one or more parameters selected from speed, diffusion coefficient, and/or straightness is indicative of infection, infectious SIRS, and/or non-infectious SIRS. Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### BRIEF DESCRIPTION OF THE FIGURES

The following figures form part of the present specification and are included to further demonstrate certain aspects of the present disclosure. The disclosure may be better understood by reference to one or more of these figures in combination with the detailed description of specific embodiments presented herein.

FIG. 1: Shows an example of a microfluidic system for leukocyte adhesive function assay (LAFA). Adhesive substrates (e.g. human VCAM-1 protein) was coated on the bottom of the microfluidic channel. Human whole blood was perfused through the channel, allowing the interaction between substrate and human leukocytes, which is detected by a fluorescence microscope. Different human leukocytes were labelled with different fluorophore conjugated antibodies against specific cell membrane markers (e.g. CD4-Alexa488, CD8-PE and CD15-APC, CD19-BV510), allowing a concurrent detection of multiple leukocyte subsets.

FIG. 2. (A) An example flow chart for conventional image and data analysis. Images captured in leukocyte adhesive function assay (LAFA) were processed and analysed using TrackMate from Fiji image analysis software, according to certain exemplary embodiments. The outputs from TrackMate were further analysed by a R program to generate descriptive statistics. The uses of the 5 scripts involved in the image analysis process was also indicated. (B) Illustrates an example flow chart for machine learning analysis based on raw images. Raw images are converted into standard deviation projections over time and used to train the algorithm to distinguish between ‘basal’ and ‘abnormal’ state. Training is usually only required the first time, subsequently, step 2b may be omitted. (C) Illustrates an example flow chart for machine learning analysis based on tracking results. TrackMate results (obtained in step 4a) are used to train the algorithm to distinguish between ‘basal’ and ‘abnormal’ state. Training is usually only required the first time, subsequently, step 2c may be omitted.

FIG. 3. Effects of Mn²⁺ treatments on α4β1 integrin adhesive function determined by LAFA on VCAM-1 substrate. Blood samples were collected from healthy volunteers, then treated with or without 5 mM MnCl₂ before being used for LAFA. Cell density (A), speed (B), diffusion coefficient (C), straightness (D), dwell time (E) and track length (F) were then determined in CD14, CD15+CD16+, CD4, CD8, CD19 and CD4+CD25 cells. Data represent mean±SEM of n=7-13 independent subjects per group. *, p<0.05; **, p<0.01

FIG. 4. Effects of Mn²⁺ treatments on α4β7 integrin adhesive function determined by LAFA on MAdCAM-1 substrate. Blood samples were collected from healthy volunteers, then treated with or without 5 mM MnCl₂ before being used for LAFA. Cell density (A), speed (B), diffusion coefficient (C), straightness (D), dwell time (E) and track length (F) were then determined in CD4, CD8 and CD15+CD16+ cells. Data represent mean±SEM of n=12 independent subjects per group. *, p<0.05; **, p<0.01

FIG. 5. Full blood cell counts in healthy controls and SIRS patients. Blood samples were collected from healthy volunteers and SIRS patients. The full blood cell counts were performed using a Mindray BC5000 Haematology Analyser according to manufacturer's instructions (A). The percentage of each leukocyte sub-populations were then determined (B) *, p<0.05; **, p<0.01.

FIG. 6. Use of LAFA to assess leukocyte adhesive function in healthy controls and SIRS patients using VCAM-1 as a substrate. Blood samples were collected from healthy volunteers and SIRS patients, and then analysed by LAFA on VCAM-1 substrate. The cell density of interacting CD14, CD15+CD16+, CD4, CD8, CD19 and CD4+CD25+ cells were determined (A). The cell density was also normalised by appropriate cell counts (B). Within the interacting cells, the percentage of specific cell sub-populations was determined in panel C. The R factor, defined as (% of cell type)/(% cell type in circulation), was calculated (D). The cell speed (E), diffusion coefficient (F), straightness (G), dwell time (H) and track length (1) of the cell sub-populations were also determined. Data represent mean±SEM of n=13 (healthy) and 14 (SIRS) independent subjects per group. *, p<0.05; **, p<0.01

FIG. 7. Use of LAFA (VCMA-1) to distinguish non-infectious SIRS patient group from infectious SIRS patient group. The 14 SIRS patients were divided into three groups based on their clinical records: 1) Non-infectious SIRS (n=6), 2) Infectious SIRS (n=5) and 3) Unknown (n=3). Blood samples were collected from healthy volunteers (n=13) and SIRS patients, and then analysed by LAFA on VCAM-1 substrate. The full blood cell counts were determined by a Mindray BC5000 Haematology Analyser (A). The blood samples were then analysed by LAFA using VCAM-1 as a substrate. With respect to interacting cells, the percentage of specific cell sub-populations was determined in panel B. The R factor, defined as (% of cell type)/(% cell type in circulation), was calculated (C). The cell density of CD14, CD15+CD16+, CD4, CD8, CD19 and CD4+CD25+ cells were determined in panel D. The cell density was also normalised by appropriate cell counts (E). The cell speed (F), diffusion coefficient (G), straightness (H) and dwell time (I) in healthy and three SIRS groups were also assessed. *, p<0.05; **, p<0.01 related to healthy controls. #, p<0.05 related to non-infectious group.

FIG. 8. A-D Illustrate the use of single cell speed profiles generated by LAFA on VCAM-1 substrate to assess specific immune response in individual SIRS patients. Blood samples were collected from SIRS patients, and then analysed by LAFA on VCAM-1 substrate. The speed of each interacting CD15+CD16+(A), CD4 (B), CD8 (C) and CD19 (D) cell was then determined. Based on the standard microbiological tests and clinical records, the causes of systemic inflammatory response in each SIRS (14 in total) were determined (Table 3). Three healthy subjects were also included as references. Each solid dot on the graphs presents a single cell. Data present mean±95% confidence interval.

FIG. 9. Use of single cell diffusion coefficient profiles generated by LAFA on VCAM-1 substrate to assess specific immune response in individual SIRS patients. Blood samples were collected from SIRS patients, and then analysed by LAFA on VCAM-1 substrate. The diffusion coefficient of each interacting CD15+CD16+(A), CD4 (B), CD8 (C) and CD19 (D) cell was then determined. Based on standard microbiological tests (blood culture tests) and clinical records, the causes of systemic inflammatory response in each SIRS patient (14 in total) were determined (Table 3). Three healthy subjects were also included as references. Each solid dot on the graphs presents a single cell. Data present mean±95% confidence interval.

FIG. 10. Use of single cell straightness profiles generated by LAFA on VCAM-1 substrate to assess specific immune response in individual SIRS patients. Blood samples were collected from SIRS patients, and then analysed by LAFA on VCAM-1 substrate. The straightness of each interacting CD15+CD16+(A), CD4 (B), CD8 (C) and CD19 (D) cell was then determined. Based on the standard microbiological tests and clinical records, the causes of systemic inflammatory response in each SIRS patient (14 in total) were determined (Table 3). Three healthy subjects were also included as references. Each solid dot on the graphs presents a single cell. Data present mean±95% confidence interval.

FIG. 11. A-I illustrate the use of LAFA to assess leukocyte adhesive function in healthy controls and SIRS patients using VCAM-1 plus IL-8 as substrates. Blood samples were collected from healthy volunteers and SIRS patients, and then analysed by LAFA on VCAM-1 plus IL-8 substrates. The cell density of interacting CD14, CD15+CD16+, CD4, CD8, CD19 and CD4+CD25+ cells were determined (A). The cell density was also normalised by appropriate cell counts (B). Within the interacting cells, the percentage of specific cell sub-populations was determined in panel C. The R factor, defined as (% of cell type)/(% cell type in circulation), was calculated (D). The cell speed (E), diffusion coefficient (F), straightness (G), dwell time (H) and track length (I) of the cell sub-populations were also determined. Data represent mean±SEM of n=13 (healthy) and 14 (SIRS) independent subjects per group. *, p<0.05; **, p<0.01.

FIG. 12. A-F illustrate the use of LAFA on VCAM-1 plus IL-8 substrates to distinguish non-infectious SIRS patient group from infectious SIRS patient group. The 14 SIRS patients were divided into three groups based on their clinical records: 1) Non-infectious SIRS (n=6), 2) Infectious SIRS (n=5) and 3) Unknown (n=3). Blood samples were collected from healthy volunteers (n=13) and SIRS patients, and then analysed by LAFA on VCAM-1 plus IL-8 substrates. The cell density of CD14, CD15+CD16+, CD4, CD8, CD19 and CD4+CD25+ cells were determined (A). The cell density was also normalised by appropriate cell counts (B). The cell speed (C), diffusion coefficient (D), straightness (E) and dwell time (I) in healthy and three SIRS groups were also assessed. Data represent mean±SEM. *, p<0.05; **, p<0.01 related to healthy controls.

FIG. 13. A-I illustrate the use of LAFA to assess leukocyte adhesive function in healthy controls and SIRS patients using VCAM-1 plus SDF-1α as substrates. Blood samples were collected from healthy volunteers and SIRS patients, and then analysed by LAFA on VCAM-1 plus SDF-1α substrates. The cell density of interacting CD14, CD15+CD16+, CD4, CD8, CD19 and CD4+CD25+ cells were determined (A). The cell density was also normalised by appropriate cell counts (B). Within the interacting cells, the percentage of specific cell sub-populations was determined in panel C. The R factor, defined as (% of cell type)/(% cell type in circulation), was calculated (D). The cell speed (E), diffusion coefficient (F), straightness (G), dwell time (H) and track length (I) of the cell sub-populations were also determined. Data represent mean±SEM of n=13 (healthy) and 14 (SIRS) independent subjects per group. *, p<0.05; **, p<0.01.

FIG. 14. A-F illustrates the use of LAFA on VCMA-1 plus SDF-1α substrates to distinguish non-infectious SIRS patient group from infectious SIRS patient group. The 14 SIRS patients were divided into three groups based on their clinical records: 1) Non-infectious SIRS (n=6), 2) Infectious SIRS (n=5) and 3) Unknown (n=3). Blood samples were collected from healthy volunteers (n=13) and SIRS patients, and then analysed by LAFA on VCAM-1 plus SDF-1α substrates. The cell density of CD14, CD15+CD16+, CD4, CD8, CD19 and CD4+CD25+ cells were determined (A). The cell density was also normalised by appropriate cell counts (B). The cell speed (C), diffusion coefficient (D), straightness (E) and dwell time (I) in healthy and three SIRS groups were also assessed. Data represent mean±SEM. *, p<0.05; **, p<0.01 related to healthy controls. #, p<0.05; ##, p<0.01 related to non-infectious group.

FIG. 15. A-I illustrate the use of leukocyte adhesive function assay (LAFA) to assess Mn²⁺ effects on leukocyte adhesive function in SIRS patients on VCAM-1 substrate. Blood samples were collected from healthy volunteers and SIRS patients and treated with 5 mM Mn for 5 minutes at room temperature, before being analysed by LAFA on VCAM-1 substrate. The cell density of interacting CD14, CD15+CD16+, CD4, CD8, CD19 and CD4+CD25+ cells were determined (A). The cell density was also normalised by appropriate cell counts (B). Within the interacting cells, the percentage of specific cell sub-populations was determined in panel C. The R factor, defined as (% of cell type)/(% cell type in circulation), was calculated (D). The Speed Activation Potential Ratio (SAPR), Diffusion Coefficient Activation Potential Ratio (DCAPR), Straightness Activation Potential Ratio (STAPR), Dwell Time Activation Potential Ratio (DTAPR) and Track Length Activation Potential Ratio (TLAPR) were then calculated (E-I), as mentioned in Example 11. Data represent mean±SEM of n=13 (healthy) and 14 (SIRS) independent subjects per group. *, p<0.05; **, p<0.01.

FIG. 16. 16A-F Illustrates the use of LAFA on VCMA-1 substrate to distinguish non-infectious SIRS patient group from infectious SIRS patient group in the presence of Mn²⁺. The 14 SIRS patients were divided into three groups based on their clinical records: 1) Non-infectious SIRS (n=6), 2) Infectious SIRS (n=5) and 3) Unknown (n=3). Blood samples were collected from healthy volunteers (n=13) and SIRS patients, and then analysed by LAFA on VCAM-1 substrate. The cell density of CD14, CD15+CD16+, CD4, CD8, CD19 and CD4+CD25+ cells were determined (A). The cell density was also normalised by appropriate cell counts (B). The Speed Activation Potential Ratio (SAPR), Diffusion Coefficient Activation Potential Ratio (DCAPR), Straightness Activation Potential Ratio (STAPR) and Dwell Time Activation Potential Ratio (DTAPR) were then calculated (C-F). Data represent mean±SEM. *, p<0.05; **, p<0.01 related to healthy controls. #, p<0.05; ##, p<0.01 related to non-infectious group.

FIG. 17. A-I illustrate the use of LAFA to assess leukocyte adhesive function in healthy controls and SIRS patients using P-selectin plus E-selectin as substrates. Blood samples were collected from healthy volunteers and SIRS patients, and then analysed by LAFA on P-selectin plus E-selectin substrates. The cell density of interacting CD14, CD15+CD16+, CD4, CD8, CD19 and CD4+CD25+ cells were determined (A). The cell density was also normalised by appropriate cell counts (B). Within the interacting cells, the percentage of specific cell sub-populations was determined in panel C. The R factor, defined as (% of cell type)/(% cell type in circulation), was calculated (D). The cell speed (E), diffusion coefficient (F), straightness (G), dwell time (H) and track length (I) of the cell sub-populations were also determined. Data represent mean±SEM of n=13 (healthy) and 14 (SIRS) independent subjects per group. *, p<0.05; **, p<0.01.

FIG. 18. Illustrates the use of LAFA on P-selectin plus E-selectin substrates to distinguish non-infectious SIRS patient group from infectious SIRS patient group. The 14 SIRS patients were divided into three groups based on their clinical records: 1) Non-infectious SIRS (n=6), 2) Infectious SIRS (n=5) and 3) Unknown (n=3). Blood samples were collected from healthy volunteers (n=13) and SIRS patients, and then analysed by LAFA on P-selectin plus E-selectin substrates. The cell density of CD14, CD15+CD16+, CD4, CD8, CD19 and CD4+CD25+ cells were determined (A). The cell density was also normalised by appropriate cell counts (B). The cell speed (C), diffusion coefficient (D), straightness (E) and dwell time (I) in healthy and three SIRS groups were also assessed. Data represent mean±SEM. *, p<0.05; **, p<0.01 related to healthy controls. #, p<0.05 related to non-infectious group.

FIG. 19. A-D illustrate the use of single cell speed profiles generated by LAFA on P-selectin plus E-selectin substrates to assess specific PSGL-1 adhesive function in individual SIRS patients. Blood samples were collected from SIRS patients, and then analysed by LAFA on P-selectin plus E-selectin substrates. The speed of each interacting CD15+CD16+(A), CD4 (B), CD8 (C) and CD19 (D) cells were then determined. Based on the standard microbiological tests and clinical records, the causes of systemic inflammatory response in each SIRS (14 in total) were determined (Table 3). Three healthy subjects were also included as references. Each solid dot on the graphs presents a single cell. Data present mean±95% confidence interval.

FIG. 20. A-D illustrate the use of single cell diffusion coefficient profiles generated by LAFA on P-selectin plus E-selectin substrates to assess specific PSGL-1 adhesive function in individual SIRS patients. Blood samples were collected from SIRS patients, and then analysed by LAFA on P-selectin plus E-selectin substrates. The diffusion coefficient of each interacting CD15+CD16+(A), CD4 (B), CD8 (C) and CD19 (D) cells were then determined. Based on the standard microbiological tests and clinical records, the causes of systemic inflammatory response in each SIRS (14 in total) were determined (Table 3). Three healthy subjects were also included as references. Each solid dot on the graphs presents a single cell. Data present mean±95% confidence interval.

FIG. 21. A-D illustrate the use of single cell straightness profiles generated by LAFA on P-selectin plus E-selectin substrates to assess specific PSGL-1 adhesive function in individual SIRS patients. Blood samples were collected from SIRS patients, and then analysed by LAFA on P-selectin plus E-selectin substrates. The straightness of each interacting CD15+CD16+ (A), CD4 (B), CD8 (C) and CD19 (D) cells were then determined. Based on the standard microbiological tests and clinical records, the causes of systemic inflammatory response in each SIRS (14 in total) were determined (Table 3). Three healthy subjects were also included as references. Each solid dot on the graphs presents a single cell. Data present mean±95% confidence interval.

FIG. 22. A-C illustrate the effects of Natalizumab on leukocyte recruitment in SIRS patients determined by LAFA on VCAM-1 substrate. Blood samples were collected from healthy subjects and SIRS patients, and treated with or without 30 μg/ml of Natalizumab at room temperature for 5 minutes before being analysed by LAFA on VCAM-1 substrate. The cell density of CD15+CD16+ (A), CD4 (B) and CD8 (C) cells was then determined. The 14 SIRS patients were divided into three groups based on their clinical records: 1) Non-infectious SIRS (n=6), 2) Infectious SIRS (n=5) and 3) Unknown (n=3). Data represent mean±SEM. NC=untreated controls, NAT=Natalizumab treated. *, p<0.05; **, p<0.01.

FIG. 23. A-B illustrate the use of serum C-reactive protein (CRP) to distinguish non-infectious SIRS patient group form infectious SIRS patient group. The 14 SIRS patients were divided into three groups based on their clinical records: 1) Non-infectious SIRS (n=6), 2) Infectious SIRS (n=5) and 3) Unknown (n=3). Blood samples were collected from healthy volunteers (n=13) and SIRS patients, and the serum CRP (A) levels were then determined by ELISA.

FIG. 24. A-F illustrate the use of LAFA to assess leukocyte adhesive function in healthy controls and SIRS patients using MAdCAM-1 as a substrate. Blood samples were collected from healthy volunteers and SIRS patients, and then analysed by LAFA on MAdCAM-1 substrate. The cell density of interacting CD4, CD8 and CD15+CD16+ cells were determined (A). The cell density was also normalised by appropriate cell counts (B). The cell speed (C), diffusion coefficient (D), straightness (E) and dwell time (F) of the cell sub-populations were also determined. Data represent mean±SEM of n=13 (healthy) and 14 (SIRS) independent subjects per group. *, p<0.05; **, p<0.01.

FIG. 25. A-I illustrate the use of LAFA (MAdCAM-1) to distinguish non-infectious SIRS patient group form infectious SIRS patient group. The 14 SIRS patients were divided into three groups based on their clinical records: 1) Non-infectious SIRS (n=6), 2) Infectious SIRS (n=5) and 3) Unknown (n=3). Blood samples were collected from healthy volunteers (n=13) and SIRS patients, and then analysed by LAFA on MAdCAM-1 substrate. The cell density of CD4, CD8 and CD15+CD16+ cells were determined (A). The cell density was also normalised by appropriate cell counts (B). The cell speed (C), diffusion coefficient (D), straightness (E) and dwell time (F) in healthy and three SIRS groups were also assessed. Data represent mean±SEM. *, p<0.05; **, p<0.01 related to healthy controls. #, p<0.05, ##, p<0.01 related to non-infectious group.

FIG. 26. A-I illustrate the use of leukocyte adhesive function assay (LAFA) to assess Mn²⁺ effects on leukocyte adhesive function in SIRS patients on MAdCAM-1 substrate. Blood samples were collected from healthy volunteers and SIRS patients and treated with 5 mM Mn²⁺ for 5 minutes at room temperature, before being analysed by LAFA on MAdCAM-1 substrate. The cell density of interacting CD4, CD8 and CD15+CD16+ cells were determined (A). The cell density was also normalised by appropriate cell counts (B). The cell speed (C), diffusion coefficient (D), straightness (E) and dwell time (F) of the cell sub-populations were also determined. Data represent mean±SEM of n=13 (healthy) and 14 (SIRS) independent subjects per group. *, p<0.05.

FIG. 27. A-F illustrate the use of LAFA on MAdCAM-1 substrate to distinguish non-infectious SIRS patient group from infectious SIRS patient group in the presence of Mn²⁺. The 14 SIRS patients were divided into three groups based on their clinical records: 1) Non-infectious SIRS (n=6), 2) Infectious SIRS (n=5) and 3) Unknown (n=3). Blood samples were collected from healthy volunteers (n=13) and SIRS patients, treated with 5 mM Mn for 5 minutes at room temperature before being analysed by LAFA on MAdCAM-1 substrates. The cell density of CD4, CD8 and CD15+CD16+ cells were determined (A). The cell density was also normalised by appropriate cell counts (B). The cell speed (C), diffusion coefficient (D), straightness (E) and dwell time (F) in healthy and three SIRS groups were also assessed. Data represent mean±SEM. *, p<0.05; **, p<0.01 related to healthy controls. #, p<0.05, ##, p<0.01 related to non-infectious group.

FIG. 28. The effects of suspected viral infection on leukocyte adhesive functions measured by LAFA using P+E selectin substrates. Blood samples were collected at three different stage of the infection: “−”, healthy, “+”, incubation period with no symptom of flu and, “++”, flu period with severe flu symptoms. The blood samples were analysed by LAFA using P+E selectin as substrate. The data represents parameters detected in individual cell tracked from the LAFA assays, which include the speed (A), diffusion coefficient (B), straightness (C), dwell time (D), track length (E) and displacement (F). Each dot presents a cell. *, p<0.05, **, p<0.01.

FIG. 29. The effects of suspected viral infection on leukocyte adhesive functions measured by LAFA using VCAM-1 as substrate. Blood samples were collected at three different stage of the infection: “−”, healthy, “+”, incubation period with no symptom of flu and, “++”, flu period with severe flu symptoms. The blood samples were analysed by LAFA using VCAM-1 as substrate. The data represents parameters detected in individual cell tracked from the LAFA assays, which include the speed (A), diffusion coefficient (B), straightness (C), dwell time (D), track length (E) and displacement (F). Each dot presents a cell. *, p<0.05, **, p<0.01.

FIG. 30. Use of LAFA to assess leukocyte adhesive function in healthy controls and SIRS patients using P+E selectins as substrates. Blood samples were collected from healthy volunteers (n=14) and 28 SIRS patients (including initial 14 plus the additional 14 patients), and then analysed by LAFA on P+E selectin substrates. The SIRS patients were grouped into non-infectious SIRS (n=11), infectious SIRS (n=10) and unknown (n=8) based on patients' clinical records and blood cultures results. The cell density of interacting CD14, CD15+CD16+, CD4, CD8, CD19 and CD4+CD25+ cells were determined (A). The cell density was also normalised by appropriate cell counts (B). The cell speed (C), diffusion coefficient (D), straightness (E), dwell time (F), track length (G) and displacement (H) of each cell sub-populations were determined. The whole white blood cell counts of the blood samples were also shown in panel L Data represent mean±SEM. *, p<0.05; **, p<0.01 related to the healthy group. #, p<0.05 related to non-infectious group. FEB stands for full blood examine.

FIG. 31. Use of single cell profiles generated by LAFA on P+E selectin substrates to assess specific immune response in individual SIRS patients. Blood samples were collected from healthy subjects (n=6) and SIRS patients (n=28), and then analysed by LAFA on selectin substrates. The straightness of each interacting CD4 (A) and CD15+CD16+ (B) cell was then determined. Based on the standard microbiological tests and clinical records, the causes of systemic inflammatory response in each SIRS patient were determined (Table 3 and Table 5). Each solid dot on the graphs presents a single cell. Data present mean±95% confidence interval.

FIG. 32. Use of LAFA to assess leukocyte adhesive function in healthy controls and SIRS patients using VCAM-1 as substrate. Blood samples were collected from healthy volunteers (n=14) and 28 SIRS patients (including old 14 and new 14 patients), and then analysed by LAFA on VCAM-1 substrates. The SIRS patients were grouped into non-infectious SIRS (n=11), infectious SIRS (n=10) and unknown (n=8) based on patients' clinical records and blood culture results. The cell density of interacting CD14, CD15+CD16+, CD4, CD8, CD19 and CD4+CD25+ cells were determined (A). The cell density was also normalised by appropriate cell counts (B). The cell speed (C), diffusion coefficient (D), straightness (E), dwell time (F), track length (G) and displacement (H) of each cell sub-populations were determined. Data represent mean±SEM. *, p<0.05; **, p<0.01 related to the healthy group. #, p<0.05 related to non-infectious group.

FIG. 33. Use of single cell profiles generated by LAFA on VCAM-1 substrates to assess specific immune response in individual SIRS patients. Blood samples were collected from healthy subjects (n=6) and SIRS patients (n=28), and then analysed by LAFA on VCAM-1 substrates. The speed of each interacting CD19 cell (A) and the straightness of each interacting CD15+CD16+ (B) cell in the blood samples were then determined. Based, at least in part, on the standard microbiological tests and clinical records, the causes of systemic inflammatory response in each SIRS patient were determined (Table 3 and Table 5). Each solid dot on the graphs presents a single cell. Data present mean±95% confidence interval.

FIG. 34. Use of LAFA to assess leukocyte adhesive function in healthy controls and SIRS patients using VCAM-1 plus IL-8 as substrates. Blood samples were collected from healthy volunteers (n=14) and all SIRS patients (including old 14 and new 14 patients), and then analysed by LAFA on VCAM-1 plus IL-8 substrates. The SIRS patients were grouped into non-infectious SIRS (n=11), infectious SIRS (n=10) and unknown (n=8) based on patients' clinical records and blood culture results. The cell density of interacting CD15+CD16+, CD4 and CD8 cells were determined (A). The cell density was also normalised by appropriate cell counts (B). The cell speed (C), diffusion coefficient (D), straightness (E), dwell time (F), track length (G) and displacement (H) of each cell sub-populations were determined. Data represent mean±SEM. *, p<0.05; **, p<0.01 related to the healthy group. #, p<0.05 related to non-infectious group.

FIG. 35. Use of LAFA to assess leukocyte adhesive function in healthy controls and SIRS patients using VCAM-1 plus SDF-1α as substrates. Blood samples were collected from healthy volunteers (n=14) and all SIRS patients (including old 14 and new 14 patients), and then analysed by LAFA on VCAM-1 plus SDF-1α substrates. The SIRS patients were grouped into non-infectious SIRS (n=11), infectious SIRS (n=10) and unknown (n=8) based on patients' clinical records and blood culture results. The cell density of interacting CD15+CD16+, CD4 and CD8 cells were determined (A). The cell density was also normalised by appropriate cell counts (B). The cell speed (C), diffusion coefficient (D), straightness (E), dwell time (F), track length (G) and displacement (H) of each cell sub-populations were determined. Data represent mean±SEM. *, p<0.05; **, p<0.01 related to the healthy group. #, p<0.05 related to non-infectious group.

### DETAILED DESCRIPTION

The present disclosure is described in further detail with reference to one or more embodiments, some examples of which are illustrated in the accompanying drawings. The examples and embodiments are provided by way of explanation and are not to be taken as limiting to the scope of the disclosure. Furthermore, features illustrated or described as part of one embodiment may be used by themselves to provide other embodiments and features illustrated or described as part of one embodiment may be used with one or more other embodiments to provide further embodiments. The present disclosure covers these variations and embodiments as well as other variations and/or modifications.

### General Techniques and Definitions

Unless specifically defined otherwise, technical and scientific terms used herein shall be taken to have the same meaning as commonly understood by one of ordinary skill in the art (e.g., in immunology, immunohistochemistry, protein chemistry, cell biology, biochemistry and chemistry).

Unless otherwise indicated, the recombinant protein, cell culture, and immunological techniques utilized in the present disclosure are standard procedures, known to those skilled in the art, such as those described in J. Perbal, A Practical Guide to Molecular Cloning, John Wiley and Sons (1984), J. Sambrook et al., Molecular Cloning: A Laboratory Manual, 3rd edn, Cold Spring Harbour Laboratory Press (2001), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including updates until present).

### Infectious Disease and Host Immune Response

An infectious disease is an illness that may be caused by the invasion of foreign pathogens and the responses from the host immune system in reaction to the invasion. The infectious pathogens include bacteria, virus, fungi, nematodes, arthropods and other macro-parasites. In 2010, an estimated 15 million people died from infectious diseases, majority of which were caused by a small group of known species of micro-organisms.

Following a successful entry into a host body, infectious pathogens grow and produce toxic reagents, leading to cell and tissue damages. The injured or effected host cells may also cause an abnormal production of cytokines and signalling molecules, some of which are released into the circulation, leading to a systemic response. At this stage, if the host immune system fails to restore the homeostasis, the systemic host immune response may be exaggerated, which may lead to more devastating consequences than the damages directly caused by the pathogens alone.

To treat patients with infectious diseases, it is useful to determine and characterise the source of infection. Subsequently, a range of antibacterial, antiviral, antifungal and anti-parasitic agents may then be used to help a patient's immune system to clear the invading pathogens. Due to the risk of uncontrolled host inflammatory response triggered by the infection, however, it is also important to accurately monitor the status of a patient immune response, so that appropriate therapies may be applied to avoid unwanted harm caused by the immune system to the host tissues.

### Leukocyte Recruitment

During the process of leukocyte recruitment, circulating leukocytes tether and roll along the endothelial surface via the interaction between leukocyte-expressed PSGL-1 (P-selectin glycoprotein ligand-1) and its endothelial ligands, P-selectin and E-selectin. Rolling leukocytes subsequently reduce their rolling velocity as a result of chemokine induced cell activation. This allows the interaction between leukocyte 132 and a4 integrins with their endothelial ligands, including intercellular adhesion molecule-1 (ICAM-1) and vascular cell adhesion molecule-1 (VCAM-1), leading to leukocyte firm adhesion on endothelial surface. Adherent leukocytes are able to, for example, use αL integrin (CD11a) and αM integrin (CD11b) to interact with endothelial ICAM-1, allowing leukocytes to crawl on the endothelial surface before finding a site for leukocyte extravasation.

Thus, an assessment of the ability of circulating leukocytes to interact with endothelial cells provides a useful tool to determine the activity of these leukocytes, reflecting the status of the host immune response. Generally, despite its causative and direct role in the disease pathogenesis, leukocyte adhesive function cannot be assessed by existing commercial tests.

### Leukocyte Adhesive Function Assay

The present disclosure provides a leukocyte adhesive function assay (LAFA). This assay, for example, allows an accurate and quantitative assessment of leukocyte adhesive function on a molecular level.

LAFA uses self-contained microfluidic/fluorescent image capture and analysis system that mimics human blood microcirculation in vitro. To study adhesive function of a specific leukocyte adhesion molecule, its corresponding endothelial ligand (also referred to herein as an “endothelial cell molecule”, or “adhesive substrate” when bound to a support or substrate) may be pre-coated on the interior surface of the microfluidic channels. Different leukocytes sub-populations may be labelled with fluorescence conjugated antibodies against specific leukocyte markers in whole blood, so that multiple subsets of leukocytes may be visualised concurrently by a fluorescent microscope. Blood may then be perfused through the microfluidic channels at a defined flow rate and the leukocyte interaction with the pre-coated endothelial molecules may then be recorded. Thus, this assay allows, for example, assessment of leukocyte adhesive function in real-time during perfusion of blood through the microfluidic channels at the defined flow rate. The recorded images may then be subsequently analysed by an algorithm. A number of cell kinetic parameters may then be used to quantitatively characterise adhesive functions of the specific subsets of leukocytes. By simply substituting the pre-coated substrates, for example, adhesive functions of other leukocyte expressing adhesion molecules may also be assessed in a similar fashion.

In the present disclosure, LAFA was employed to identify new markers to assess host immune response in subjects with an inflammatory immune response, for example such as SIRS patients. A range of new markers were generated by LAFA, which may then be used to determine the different inflammatory immune responses in individual patients. The results disclosed herein indicate LAFA may serve as a useful tool to distinguish patients based on specific causes inflammation, facilitating the development of optimal therapies on personal basis.

The leukocyte adhesive function assay may be various suitable types of assay. The method may comprise carrying out more than one leukocyte adhesive function assay, to obtain one or more results. The leukocyte adhesive function assay may include one or more specific tests to provide a collective result.

In certain exemplary embodiments, the leukocyte adhesive function assay results may be semi-quantitative and/or quantitative.

The leukocyte adhesive function assay may achieve one or more of the following: characterising leukocyte cell recruitment; characterising leukocyte cell tracking; and characterising leukocyte cell migratory behaviour—in a semi-quantitative or quantitative manner.

In some embodiments, the leukocyte adhesive function assay may entail quantitatively determining leukocyte migration. This may include detecting, measuring or observing one or more of the following: leukocyte cell tethering, rolling, slow rolling, firm adhesion, crawling and transendothelial migration. In some embodiments, the leukocyte adhesive function assay may entail detecting, measuring or observing one or more of the following: leukocyte cell average speed, displacement, acceleration, deceleration, directionality, dwell time and straightness.

Interacting leukocytes may be characterised by way of velocity distribution. For example, interacting leukocytes may be divided into five interaction types according to cell mean speed (Sₘₑₐₙ): static cells (Sₘₑₐₙ<5 μm/min), crawling cells (Sₘₑₐₙ=5-20 μm/min), slow rolling cells (Sₘₑₐₙ=20-300 μm/min), and rolling cells (Sₘₑₐₙ=300-6000 μm/min). In addition, a histogram may be used to show the distribution of cell velocity.

In certain embodiments, the leukocyte adhesive function assay entails detecting, measuring and/or observing leukocyte migration under realistic physiological conditions.

In some embodiments the assay allows for simultaneous detection of different leukocyte subsets.

In certain embodiments, the leukocyte adhesive function assay involves a flow assay.

As part of the leukocyte adhesive function assay, the blood sample may be premixed, pre-treated or pre-incubated with one or more cell stains, one or more chemicals (e.g. such as manganese which induces a4 integrin activation), one or more drugs (with or without a detectable moiety), one or more antibodies, and/or one or more detectable moieties or other reagents or agents.

In some embodiments, the method may comprise treating subject (human or animal) blood with one or more drugs, reagents or agents in vitro, then carrying out the leukocyte adhesive function assay.

In some embodiments, the leukocyte adhesive function assay may assess leukocyte migration under realistic physiological conditions.

In some embodiments, the leukocyte adhesive function assay may utilise leukocytes labelled with an antibody conjugated to a fluorophore or other detectable moiety. In some embodiments, the assay may entail detecting different subsets of leukocytes with subset-specific antibodies conjugated to different fluorophores. For example, an antibody or antibody cocktail and/or stain may be added to the blood sample. For example, fluorescently labelled antibodies against specific leukocyte membrane markers may be added to the blood sample before performing a flow assay.

The leukocyte adhesive function assay or flow assay may utilise a suitable type of equipment for detecting, measuring or observing leukocyte migration etc, including for detecting, measuring or observing leukocyte migration etc under realistic physiological conditions. Examples of suitable microfluidic assays and/or devices are described in the following documents: U.S. Pat. Nos. 8,940,494; 8,380,443; 7,326,563; WO 92/21746; Vaidyanathan (2014)—the entire contents of each of these references are herein incorporated by reference in their entirety.

A microfluidic device may be used for carrying out a flow assay. In some embodiments, the flow assay entails using a microfluidic device having one, two, three, four, five, six or more microfluidic channels, for example, for detecting different leukocyte subsets and/or adhesion molecules.

In some embodiments, the blood sample may be assayed in a microfluidic device to mimic blood flow in vivo.

In some embodiments, the flow assay entails pulling or pushing the blood sample into one or more microfluidic channels, for example using a syringe pump, such as pulling or pushing the blood sample into one or more microfluidic channels at a shear stress of approximately 0.5 to 300 dyne/cm², including 0.2, 0.5, 1, 2, 3, 4, 5, 6, 7, 8, 9, 10, 15, 20, 25, 30, 40, 50, 60, 70, 80, 100, 150, 200, 300 dyne/cm².

The leukocyte adhesive function assay may allow for visual analysis for characterising leukocyte cell migratory behaviour, characterising leukocyte cell tracking, or characterising leukocyte cell recruitment by the endothelial adhesion molecule. Visual analysis may be carried out in a suitable way. For example, visualisation may be achieved using a microscope and image recorder (e.g. video or time-lapsed photography). Leukocyte migratory behaviour, tracking, recruitment etc may be analysed by way of computer analysis of the images captured by the image recorder. The kinds and numbers of adhesive and/or non-adhesive leukocytes may be determined and their individual velocities/behaviours may be recorded and analysed in a quantitative manner.

In some embodiments, the leukocyte adhesive function assay entails acquiring images at high frame rate over a period of time sufficient to capture leukocyte cell interactions. For example, the assay may entail acquiring images at 2 frames per second for 5 minutes to capture types of cell interactions. In some embodiments, the leukocyte adhesive function assay may entail capturing detailed 3D movement of leukocytes. In some embodiments, the leukocyte adhesive function assay entails recording a fluorescence microscopy time series.

The leukocyte cell kinetic parameters may be derived in the following manner: The recorded image time series provides x, y and z (position) and t (time) coordinates of the detected, or a substantial portion of the detected, interacting leukocyte cells. By linking localizations of the same leukocyte cell between several frames using mathematical algorithms such as ‘nearest neighbour’, cells may be tracked over time and various parameters obtained to characterize cell motion (such as one or more of the following: track direction, length, displacement, duration, straightness, mean speed, acceleration/deceleration, directed and/or confined and/or random motion type). Those parameters may then be used to differentiate motility behaviour of different leukocyte cell subpopulations or changes in motility upon drug treatment.

Alternatively, other methods for detecting leukocyte cells may be used, as described for example in: Nan Sun et al. (2012), the entire contents of which are incorporated herein by reference in their entirety.

The endothelial cell molecule may be in the form of, for example, a recombinant protein bound to a support or substrate. In some embodiments, the assay involves using a plurality of endothelial molecules fixed to a support or substrate (perhaps including a lipid bilayer), and in other embodiments the assay may involve using actual cells expressing such endothelial cell molecules. With regard to endothelial cell molecules immobolised to a support or substrate, a number of techniques are referenced, for example, in Kim and Herr (2013), and is hereby incorporated by reference in its entirety. Also, such molecules are described in the following documents, the entire contents of which are incorporated herein by way of reference: U.S. Pat. Nos. 8,940,494; 8,380,443; 7,326,563; and WO 92/21746.

Endothelial cell molecules that may be used as adhesive substrate (i.e., bound to a support or substrate) in the leukocyte adhesive function assay include, but are not limited to one or more of the following:

1. Adhesion molecules as already described herein;

2. Chemokines as mentioned herein; and

3. Purified antigens and artificial antigen-presenting cell system:

a. Purified antigens: i) alpha, beta and epsilon toxins and ii) antigen CFA/I

b. Artificial antigen-presenting cell systems, such as those disclosed in 1) Thomas et al. (2002) and 2) Turtle et al. (2010), each of which is hereby incorporated by reference in its entirety;

4. Other molecules (including proteins) that may regulate cell-cell interactions; and

5. Chemokine receptors as disclosed herein.

In some embodiments, the leukocyte adhesive function assay may entail detecting, measuring or observing the interaction between leukocyte-expressed PSGL-1 (P-selectin glycoprotein ligand-1) and its endothelial molecule, P-selectin and/or E-selectin.

In some embodiments, the leukocyte adhesive function assay may entail quantitative assessment of a4 integrin adhesion functions.

In some embodiments, the leukocyte adhesive function assay may entail detecting, measuring or observing increased leukocyte a4 integrin expression and activity.

In some embodiments, the leukocyte adhesive function assay may entail measuring, detecting and/or observing the interaction between leukocyte a4 integrin and endothelial VCAM-1.

In some embodiments, the leukocyte adhesive function assay may entail detecting, measuring and/or observing the interaction between CD11α (αL integrin) and ICAM-1.

In some embodiments, the leukocyte adhesive function assay may entail detecting, measuring or observing the interaction between CD11b (αM integrin) and ICAM-1.

In some embodiments, the leukocyte adhesive function assay may entail detecting, measuring and/or observing the interaction between α4β7 integrin and MAdCAM-1.

In some embodiments, the leukocyte adhesive function assay may entail detecting, measuring and/or observing the interaction between intercellular adhesion molecule-1 (ICAM-1) and/or vascular cell adhesion molecule-1 (VCAM-1) and their leukocyte adhesion molecule.

In some embodiments, the leukocyte adhesive function assay may entail detecting, measuring and/or observing the interaction between leukocyte 132 integrin and its endothelial molecule.

The leukocyte adhesive function assay may entail measuring one or more specific subsets of leukocytes, such as CD4, CD8 and CD15 cells.

In some embodiments, the leukocyte adhesive function assay may entail detecting, measuring or observing leukocyte migratory behaviours on cytokine or chemokine (e.g. TNFα and IL-4) activated primary endothelial cells (e.g. HUVEC) or immobilised endothelial cell lines (e.g. human microcirculation endothelial cells (HMEC)).

In some embodiments, the leukocyte adhesive function assay may entail simultaneously detecting, measuring and/or observing different leukocyte subsets by labelling the subsets with specific membrane markers. Such markers may be antibodies conjugated to different fluorophores.

The leukocyte adhesive function assay may include one or more controls. The nature of the controls employed may depend on the nature of the assay and the nature of the method employing the essay. For example, the control may be a blood sample obtained from a healthy individual who does not have a disease or disorder (e.g. an inflammatory or infectious disease). For example, the control may be a blood sample obtained from an individual who is not under medical treatment with drugs (e.g. anti-inflammatory drug). For example, the control may be a blood sample obtained from the subject prior to being administered the drug, prior to receiving drug treatment, or prior to being subjected to a dosage regimen or during a dosage regimen. The control may be a blood sample comprising pooled blood samples from different individuals (cohort).

In some embodiments, the method/leukocyte adhesive function assay may entail carrying out the following steps: 1. Pre-coating a flow channel with an endothelial molecule; or if in endothelial cell models, seed and culture cells in the flow channel, and activate the expression of endothelial adhesion molecules by treating the cells with a reagent or inflammatory cytokines or chemokines, e.g. TNFα; 2. Incubating the flow channel without or with a drug at various doses (e.g. small molecule, antibody etc), which alters endothelial adhesion molecule functions; 3. Collecting blood from a subject; and, 4. Performing leukocyte adhesive function assays at various time points post-drug treatment to determine the drug effects (by comparison to drug-free controls).

### Leukocytes and Adhesion Molecules

Leukocytes include, but are not limited to, one or more of the following: neutrophils, eosinophils, basophils, CD4 T lymphocytes, CD8 T lymphocytes, T regulatory cells, B lymphocytes, dendritic cells, monocytes and natural killer cells.

Leukocyte adhesion molecules or other binding molecules of the leukocyte include one or more of the following: selectins, integrins, chemokines, chemokine receptors and others types of molecules. Thus, leukocyte adhesion molecules include, but are not limited to one or more of the following: PSGL-1, L-selectin, α1 integrin, α2 integrin, α3 integrin, α4 integrin, α5 integrin, α6 integrin, α7 integrin, α8 integrin, α9 integrin, α10 integrin, α11 integrin, αD integrin αE integrin, αV integrin, αX integrin, CD11α (αL integrin), CD11b (αM integrin), β1 integrin, β2 integrin, β4 integrin, β5 integrin, β6 integrin, β7 integrin β8 integrin, CD44, ESL-1, CD43, CD66, CD15s and ALCAM.

### Endothelial Cell Molecules

Endothelial cell molecules include one or more of the following: selectins, cell adhesion molecules (CAMs), chemokines, chemokine receptors and other types of molecules. Thus, endothelial molecules include one or more of the following: E-selectin, P-selectin, VCAM-1, ICAM-1, ICAM-2, MadCAM-1, PECAM, GlyCAM-1, JAM-A, JAM-B, JAM-C, JAM-4, JAM-L, CD34, CD99, VAP-1, L-VAP-2, ESAM, E-LAM, cadherins, and hyaluronic acid.

### Blood Sample

In some embodiments, the leukocyte adhesive function assay requires a volume of whole blood of between about 5 to about 1000 μl, such as 5, 10, 15, 20, 30, 40, 50, 60, 70, 80, 90, 100, 120, 150, 200, 300, 400, 500, 750 and 1,000 μl. The method may comprise subjecting more than one blood sample obtained from the subject to a leukocyte adhesive function assay or more than one leukocyte adhesive function assay.

The method may include the step of isolating the blood sample from the subject. This may be achieved in various suitable ways. For example, blood may be obtained by pricking a finger and collecting the drop/s, or by venepuncture. In certain embodiments a drop of blood may be used for the method. In certain embodiments, less than about 100 μL of blood may be required for the leukocyte adhesive function assay, such as 5, 10, 15, 20, 30, 40, 50, 60, 70, 80, 90, or 100 μL. In certain embodiments, less than about 100 μL of blood may be required for the leukocyte adhesive function assay, such as less than 5, 10, 15, 20, 30, 40, 50, 60, 70, 80, 90, or 100 μL.

In some embodiments, the blood sample may be whole blood, whether processed or not. In some other embodiments the blood sample is a processed sample whereby one or more components of whole blood have been separated from each other. That is, in some embodiments the blood sample may be whole blood, and in other embodiments the blood sample may comprise or one or more white blood cell components of (processed/treated) whole blood. In one embodiment, the blood sample is plasma.

In certain embodiments, blood components are not separated from the whole blood sample so as to mimic blood in vivo. In certain embodiments isolated blood cells, cultured blood cells and/or blood cell lines may be used.

Anticoagulants that may be used to collect and store blood samples may include, but are not limited to, heparin, EDTA, ACD, citrate, Hirudin, sodium polyanethol sulfonate and potassium oxalate/sodium fluoride.

### Subject

The subject may be a mammal or other suitable type of animal. Mammals include humans, primates, livestock and farm animals (e.g. horses, sheep and pigs), companion animals (e.g. dogs and cats), and laboratory test animals (e.g. rats, mice and rabbits). In certain embodiments, the subject is human

### Treating a Subject

The subject may be treated in a conventional way known for that particular disease. In addition, the subject may be treated in non-conventional way. For example, based on the results from LAFA analysis, the suitability of a drug for the treatments of a subject with certain diseases may be projected, even though the drug is usually not used for this particular disease.

In one embodiment, the method comprises determining, based at least in part on at least one LAFA, that the subject has a non-infectious inflammatory immune response, and treating the subject for the non-infectious inflammatory immune response. The treatment may comprise administering an anti-inflammatory composition to the subject. Examples of anti-inflammatory compositions include non-steroidal anti-inflammatory drugs, including Celecoxib, Etoricoxib, Ibuprofen, Ketoprofen, Naproxen, Sulindac and/or combinations thereof, as well as corticosteroids. The anti-inflammatory composition may be administered by a number of routes in accordance with accepted medical practice. Preferred modes include intravenous, intramuscular, subcutaneous and percutaneous administration, using techniques that are known in the art. Other routes of administration may be envisioned. In the case of treatment of acute inflammatory conditions that are localized, non-systemic administration may be preferred in which case the administration of the therapeutic composition is at or around the site of the acute inflammation.

In one embodiment, the method comprises determining, based at least in part on at least one LAFA, that the subject has an infectious inflammatory immune response, and treating the subject for the infectious inflammatory immune response. Treating the subject for the infectious inflammatory immune response may include treating the subject with a suitable anti-infective agent such as an antimicrobial drug or an antiviral drug. Antibiotics for treating bacterial infections are well known in the art and include penicillins, cephalosporins, polymyxins, rifamycins, lipiarmycins, quinolones, sulphonamides, macrolides, lincosamides, tetracyclines and aminoglycosides.

In one embodiment, the method comprises determining that the subject has sepsis (i.e. the presence of infection in a SIRS patient) and treating the subject with one or more of an antibiotic, vasopressor and corticosteroid. Suitable steroids include, but are not limited to, Budesonide, Cortisone, Dexamethasone, Methylprednisolone, Prednisolone, Prednisone and/or combinations thereof.

### Determining the Cause of Inflammation

In patients with infectious diseases, the immune system is highly activated due to the invasion of foreign pathogens, leading to an elevated inflammation and increased leukocyte adhesive functions.

In one embodiment, the method described herein comprises subjecting a blood sample from a subject to at least one leukocyte adhesion function assay (LAFA) and, based at least in part on the results of the at least one LAFA, determining the cause of inflammation in the subject. The cause of inflammation may be determined by analysing cell kinetic parameters in the LAFA assay. In some embodiments, the LAFA assay includes analysing leukocyte cell subsets by detecting leukocyte cell markers.

In one embodiment, the method comprises determining an infectious cause or a non-infectious cause of inflammation. In some embodiments, the method comprises determining that the infectious cause of inflammation is a bacterial, viral or parasitic infection. In one embodiment, the method comprises determining the family, genus or species of bacteria, virus or parasite.

Infectious diseases, include but are not limited to: ICAM-1 mediated infections such as rhinoviral infection, Amoebic meningoencephalitis, Acute rheumatic fever, Anthrax, atypical mycobacterial disease, Avian influenza (Bird Flu), Babesiosis, Bacterial vaginosis, Balanitis, Barmah Forest virus infection, Blastocystis infection, Botulism, Brucella infection, Campylobacter infection, Chickenpox and shingles, Chikungunya virus, Cold sores (herpes simplex type 1), Common cold, Conjunctivitis, Cryptosporidium infection, Cytomegalovirus (CMV) infection, Dengue fever, Giardia infection, Glandular fever, Gonorrhoea, Haemophilus influenzae type b (Hib), Hepatitis, Hand, foot and mouth disease, Hendra virus infection, Hydatid disease, Human papilloma virus (HPV), genital warts & related cancers, Japanese encephalitis, Kunjin/West Nile virus infection, Kunjin/West Nile virus infection, Leprosy, Legionella pneumophila infection, Leptospirosis, Listeria infection, Lyme disease, Measles, Meningococcal infection, Molluscum contagiosum, Mumps, Mycoplasma genitalium infection, Mycoplasma pneumoniae infection, Middle East respiratory syndrome (MERS), Non-specific urethritis (NSU), Norovirus infection, Parvovirus B19 infection, Plague, Pneumococcal infection, Poliovirus infection, Psittacosis, Q fever, Rabies virus and Australian bat lyssavirus, Respiratory syncytial virus (RSV) infection, Rickettsial infections, Roseola, Ross River virus infection, Rotavirus infection, Rubella, Salmonella infection, School sores, Severe acute respiratory syndrome, Shiga toxin producing Escherichia coli (STEC) and haemolytic uraemic syndrome (HUS), Shigella infection, Smallpox, Staphylococcus aureus including methicillin resistant Staphylococcus aureus (MRSA), Streptococcal sore throat, Syphilis, Tetanus, Thrush, Toxic shock syndrome, Toxoplasma infection, Trichomonas infection, Tuberculosis, Tularaemia, Typhoid and paratyphoid, Urinary tract infection, Vibrio parahaemolyticus infection, Viral gastroenteritis, Viral haemorrhagic fevers, Viral meningitis, Viral respiratory infections, Warts, Whooping cough, Worms, Yellow fever, Yersinia infection, Yersinia infection, Zika virus infection, or combinations thereof.

In one particular embodiment, the bacterial infection is selected from an infection caused by one or more of an enteric bacterium, Serrati sp., Pseudomonas sp., E. coli, and Staphylococcus sp.

In another embodiment, the method comprises determining that the non-infectious cause of inflammation is cardiovascular disease, asthma, haemorrhage, aneurism or pneumonitis. Other diseases and clinical correlates of undesirable inflammatory responses including those associated with hemolytic anemia, hemodialysis, blood transfusion, certain hematologic malignancies, pneumonia, post-ischemic myocardial inflammation and necrosis, barotrauma (decompression sickness), ulcerative colitis, inflammatory bowel disease, atherosclerosis, cytokine-induced toxicity, necrotising enterocolitis, granulocyte-transfusion-associated syndromes, Reynaud's syndrome, multiple organ injury syndromes secondary to septicemia or trauma, acute purulent meningitis, other central nervous system inflammatory disorders, or combinations thereof.

### Machine Learning

As mentioned above, the LAFA may provide video data of the cells under examination and machine learning (ML) may be applied to the video data. The video data may be 2048×2048 pixels at a frame rate of 50f/s or may be 682×682 pixels at the same or different framerate. It should be noted, however, that other resolutions, including rectangular set-ups, with different frame rates are suitable. The resolution of each frame may be reduced by downsampling and/or summation of neighbouring pixels, such as 3×3 blocks, to increase intensity and therefore sensitivity while reducing computational complexity.

Maximum intensity projection may be applied to 3D data where the 3D data comprises multiple 2D layers, such as images of slices of o CT or MRI scan. The 3D data is projected onto a 2D space by selecting, for each 2D pixel location, the maximum intensity across the 2D layers for that 2D pixel location. In the method described herein, the third dimension is the time dimension in the sense that each image of the video constitutes one 2D layer. In other words, the frames of the video are overlaid to create one single image and the maximum intensity across other frames is chosen as the intensity for that pixel of the output image. As a result, if a cell does not move because it is activated, the single image will show a bright dot at a constant cell location. Conversely, if the cell moves because it is not activated, the single image will show a line along the path of movement. This may, of course, be a curved line if the movement is non-linear.

If the cell moves slowly relative to the frame rate of the video, the line will be solid because the dots representing the cell at respective times overlap. On the other hand, if the cell moves fast relative to the frame rate of the video, the line will be dotted because the dots representing the cell at respective times are spaced apart from each other as the cell moves by more than one cell diameter between frames.

This projection on a single 2D image is particularly useful when combined with a convolutional neural network (CNN) because CNNs can exploit the structural features in the image. In particular, CNNs work on layers of the image where the first layer may be overall brightness. For activated cells, the brightness would be higher as more cells stick to the substrate. Therefore, the ML can use the brightness as an indicator for cell activation. In the second layer, the CNN may consider line features and if there is a line due to movement of the cells, the ML may take this as an indication that the cells are not activated.

While this description is relatively specific about the structural feature of the single image, it is noted that this does not need to be known in order to perform the method. The training phase of the CNN on samples that have been combined into single images, will automatically adapt the CNN to these features as the best parameters to be used to distinguish between an infectious and non-infectious inflammatory immune response.

In another example, the LAFA video data is used to perform cell tracking by using TrackMate, for example. Each of the cell tracking output parameters, such as cell density (i.e. number of spots), speed, diffusion coefficient, straightness, dwell time (i.e. duration), track length (i.e. displacement) etc. may be used as a machine learning feature. The different features may be combined in a machine learning process, such as a random forest. A random forest comprises multiple trees, which are graph structures with nodes and edges. Each node represents one feature (i.e. cell tracking parameter) connected by edges to define a decision pathway. The trees are created by feature selection during the training phase. During evaluation phase, the output of each tree is combined with the outputs of the other trees to provide a final classifier.

While it is noted that activated and non-activated cells may be classified based on a single parameter, such as speed, this may not be the case with other applications, such as other diseases. The more diseases are to be classified/distinguished or the more similar the diseases are in their feature values, the more parameters/dimensions may be used. This is where the power of the random forests becomes important because it may include a large number of parameters without the problems of overfitting.

In one example, the instantaneous speeds are calculated based on positions of cells in subsequent images. The speed values can then be used to calculate aggregated values, such as mean, median, maximum, minimum, high speeds (e.g. above median), high+ speeds (e.g. above 70%), fluctuation, total fluctuation and positive fluctuation. These values may be calculated only for tracks that are longer than a predefined threshold, such as 10 frames. It is also possible to calculate a logarithm of the speed, such as logarithm to base 10 (or other base), to convert the speed values into negative numbers for speeds less than 1 and positive numbers for speeds greater than 1. The number of times that the speeds in logarithmic scale base 10 change from positive to negative divided by the time that a recorded cell (numbers of frames where it is detected) is then used as the fluctuation feature.

In summary, there are 13 features available: mean, median, maximum, minimum, high speeds, high+ speeds, fluctuation, total fluctuation, positive fluctuation, number of spots, duration, displacement and initial mean. These features can be determined for each of short tracks (10-20 frames), medium tracks (21-50 frames) and long tracks (>50 frames). This results in 39 features. In addition, there are mean, maximum, minimum of all tracks and the number of tracks, resulting in a total of 43 features. This analysis can now be repeated for different cell types, such as CD4, CD8 and CD19, resulting in 43*3=129 features per patient.

Training data can be generated by diagnosing patients and labelling the corresponding features. This training data can then be used to randomly select seed samples to create a random forest. Each tree in the random forest has feature variables depending on the seed for that tree. The random forest can then be trained using the entire training set. That is, the different subsets of the training data are used to select the features in respective Random Forests and the actual training is then performed using the entire training set.

Existing random forest implementations can be used to optimise feature selection, seeding and training such as the sklearn.ensemble.RandomForestClassifier from scikit-learn (cikit-learn.org) or the Java implementation http://wekasourceforge.net/doc.dev/weka/classifiers/trees/RandomForest.html.

### EXAMPLES

### Example 1. Methods and Materials

### Protocol for Leukocyte Adhesive Function Assay (LAFA)

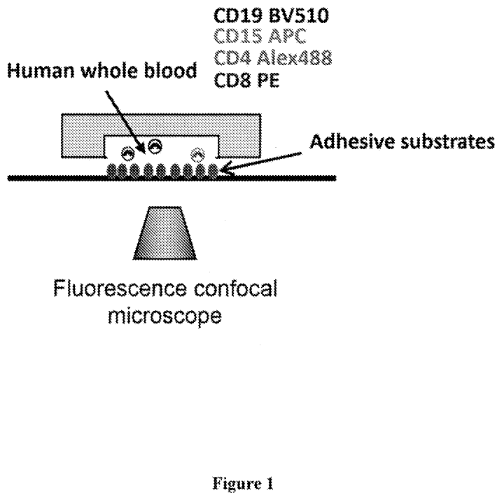

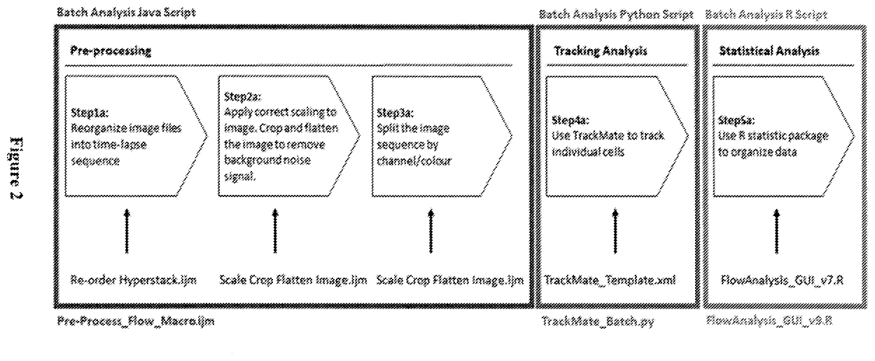

Leukocyte adhesive function assay (LAFA) quantitatively assesses the ability of leukocytes to interact with other proteins and/or molecules (e.g. adhesion molecules, chemokines and/or related peptides, which are referred to adhesive substrate in the assay), under flow conditions. The interacting leukocytes are typically visualised by labelling with fluorescence-conjugated antibodies against specific markers on leukocyte membranes, so that the cells may be detected by a fluorescent microscope (FIG. 1). Examples of antibodies used for the detection of specific leukocyte subsets are listed in Table 1. These antibodies may be used in combination or individually. In some experiments, fluorescence-conjugated antibodies against other membrane proteins may also be used to assess the expression levels of these cell surface proteins.

TABLE 1
Examples of membrane markers used to
identify specific leukocyte subsets.

| Markers | Leukocyte subsets |
|---|---|
| CD4 | CD4+ cells |
| CD8 | CD8+ cells |
| CD15, CD16 | Neutrophils (CD15+CD16+) |
| CD15, CD16 | Eosinophils (CD15+CD16−) |
| CD14, CD16 | CD14+CD16− Classical Monocytes |
| CD14, CD16 | CD14+CD16+ Non-classical Monocytes |
| CD19 | CD19+ B cells |
| CD4, CD25 | CD4+CD25+ lymphocytes |
| CD8, CD25 | CD8+CD25+ lymphocytes |

To mimic blood microcirculation in vitro, a microfluidic system is used, which consists of a microfluidic pump and microfluidic chips/channels. Adhesive substrates are pre-coated on the bottom of microfluidic channels, and leukocytes are then drawn into the channels by a microfluidic pump, allowing leukocytes to interact with pre-coated adhesive substrates (FIG. 1). These interactions are recorded by a fluorescence microscope, and the images analysed using image analysis software. As a result, the cell interaction behaviours may be described using a range of cell kinetic parameters, from which the ability of leukocytes to interact with specific adhesive substrate may be quantitatively assessed. Additionally, to assess the expression of leukocyte membrane proteins, the fluorescence intensity of antibodies against these proteins on the interacting leukocytes may be assessed with the same fluorescent microscope.

### Reagents and Adhesive Substrates

a) The proteins that were used as adhesive substrates for LAFA are listed in Table 2. These substrates may be used in combination or individually. The stocks were stored at −80° C. and discarded after their storage time as suggested by the manufacturers. No repeated freeze-thaw is allowed.

TABLE 2
Adhesive substrates used for LAFA. Coating
and stock concentrations are indicated.

| | Coating | | | | Stock | |
|---|---|---|---|---|---|---|
| | Concen- | | Manufac- | | concen- | |
| Proteins | trations | | turers | Cat# | trations | |
| Hu VCAM-1 | 10 | μg/ml | R&D | ADP5 | 1 | mg/ml |
| Hu MAdCAM-1 | 14 | μg/ml | Origene | TP319060 | Various | |
| Hu IL-8 | 1 | μg/ml | R&D | 208-IL | 0.1 | mg/ml |
| Hu SDF-1α | 1 | μg/ml | R&D | 350-NS | 0.1 | mg/ml |
| Hu E-selectin | 0.5 | μg/ml | R&D | ADP1 | 1 | mg/ml |
| Hu P-selectin | 10 | μg/ml | R&D | ADP3 | 1 | mg/ml |
| Hu ICAM-1 | 10 | μg/ml | PeporTech | 150-05-50 | 1 | mg/ml |

b) Hanks' balanced salt solution (HBSS) (Sigma, Cat #: H1387) One package of HBSS powder is reconstituted into a 1 L water, and stored at 4° C. fridge.

c) Microfluidic chips: (Microfluidic ChipShop, Cat #: 01-0178-0152-01), PMMA, Lid thickness (175 μm), Straight channel chip (16 parallel channels), Mini Luer interface, Width (1,000 μm)/Depth (200 μm)/Length (18 mm).

d) Chip inlets:
i. Mini luer to luer adapter: may hold up to 70 μl
ii. Mini luer to luer plus 500 μl tank; may hold up to 500 μl

e) MnCl₂, (Sigma, Cat #:450995)

Make 0.5 M stock, use 1:100 dilution in whole blood (5 mM final concentration).

f) Fluorescent conjugated antibodies to detect specific leukocyte subsets
i. Anti-CD4-Alexa488 (BD, Cat #: 557695)
ii. Anti-CD8-PE (BD, Cat #: 555635)
iii. Anti-CD15-APC (BD, Cat #: 551376)
iv. Anti-CD19-BV510 (BD, Cat #: 562947)
v. Anti-D16-BV510 (BD, Cat #: 360723)
vi. Anti-CD25-APC (BD, Cat #560987)

### Microfluidic Chip Preparation

a. Thaw protein stocks as needed from a −80° C. freezer, and dilute it to the coating concentrations with HBSS, as indicated in Table 2.
b. Use 15 μl of the diluted solution to pre-coat each microfluidic channel, at 4° C. overnight.
c. The first channel on the chip is left empty for auto-focusing on the InCell.
d. On the following day, the channels are washed with HBSS once before being used for LAFA.

### Blood Collection

a. 7-10 ml whole blood is collected via venepuncture, with 2 ml collected in an EDTA tube (for Full Blood Cell Counts) and 5 ml collected in lithium heparin tubes (for LAFA).
b. If a butterfly needle is used for blood collection, blood is collected in an EDTA tube and collected in a heparin tube, for example, 2 ml of blood is collected in the EDTA tube, and 5 ml of blood is collected in the heparin tube.
c. After collection, blood tubes are stored at room temperature (−20° C.) and used within 8 hours of collection.
d. Avoid vigorous shaking of the blood tubes, as it may activate the blood cells.

### Blood Pre-Treatment and Labelling

a. For each assay, 130 μl heparinised blood is required.
b. In some experiments, blood needs to be activated by 5 mM Mn for 5 min at room temperature (RT), before being used for the assay
c. The following markers may be added alone or in one or more of the following combinations to the whole blood, incubating for 5 min at RT:
   Anti CD4-Alexa488 (2 μl/100 μl whole blood)
   Anti CD8-PE (1.5 μl/100 μl whole blood)
   Anti CD15-APC (3 μl/100 μl whole blood)
   Anti CD19-BV510 (2 μl/100 μl whole blood)
   Anti CD16-BV510 (0.2 μl/100 μl whole blood)
   Anti CD25-APC (0.15 μl/100 μl whole blood)
d. If testing drug effects, the drug needs to be added to the blood and incubated before the assay. Depending on the nature of the drugs, the time and temperature required for the incubation may differ. In Mn experiments, the drug needs to be added at least 5 min after the Mn treatment.

### LAFA Assay

a. The chip is placed into the slide holder of the InCell 2200
b. Turn on both top and bottom heaters to 39° C. (which gives 35.5° C. to the slide)
c. Load the blood sample to the inlet of the chip
d. Connect the outlet of the chip to the microfluidic pump
e. Open up the protocol in the InCell software
f. Find the focus
g. Start the pump/blood perfusion at 0.6 ml/hour, using a 10 ml syringe with 16 gauge needle, providing a sheer stress of 1.5 dyn/cm².
h. Commence recording

### Video Analysis

a. Open Fiji open-source image analysis software. Download and install the version suitable for the operating system utilized prior to starting the analysis if Fiji has not been used before (https://imagej.net/Fiji/Downloads).
b. Add ‘TrackMate_-3.6.1-SNAPSHOT-sources.jar’, ‘TrackMate_-3.6.1-SNAPSHOT-tests.jar’ and ‘TrackMate_-3.6.1-SNAPSHOT.jar’ to the Fiji>Plugins folder prior to tracking analysis.
c. Open macro ‘Pre-process_Flow.ijm’ by dragging and dropping into Fiji.
d. Set number of channels and specify channels according to the file name orders.
e. Click ‘Run’, choose the directory of the folder.
f. When prompted, adjust ROI to include the channel centre and exclude channel edges. Press ‘OK’.
g. When the macro completes, a set of ‘TIF’ file from individual channel for each experiment may be generated.
h. For experiments requiring analysis with inclusion or exclusion criteria, run creating new channel macro.
   CD15+CD16+, CD15+CD16-, run macro
   Create_CD15+CD15+_CD15+CD16−_Channel.ijm’
   CD4+CD25+, run macro ‘Create_CD4-25_Channel.ijm’
i. To track cells, drag ‘TrackMate_Batch.py’ file into the Fiji.
j. Specify the directory of the tracking template file ‘Expt1_anti-flow.xmr in line 33 and image folder in line 69.
k. When the run complete, check the tracking information by going to Fiji>Plugins>Tracking>Load a TrackMate file. Then select the tracking file to make sure the macro completed correctly.
l. TrackMate .csv files may then be analysed in R.

### Secondary Data Analysis

a. Open R
b. Open “FlowAnalysis_GUI_v9.R”.
c. Go to the “FlowAnalysis” window
d. Choose “Batch” from the “Analysis Type” menu
e. Click on “Browse Working Directory” to choose the fold where the files to be analysed are located.
f. Click on the folder containing tracking data obtained from the previous steps, then press ‘OK’
g. Press “Batch” button.

The parameters may be generated in a summary table, and individual cell data for each parameter may be generated in a separate spreadsheet.

### Example 2. Microfluidic System for Leukocyte Adhesive Function Assay (LAFA)

The present example is directed to a microfluidic system for leukocyte adhesive function assay. LAFA is a self-contained microfluidic/fluorescent image capture and analysis system that mimics human blood microcirculation in vitro.

As shown in FIG. 1, microfluidic channels are pre-coated with adhesive substrates, such as endothelial adhesion molecules and chemokines. Leukocytes are labelled with different fluorophores conjugated antibodies against specific leukocyte membrane markers (e.g. CD4, CD8 and CD15), allowing simultaneous detection of these leukocyte subsets. Leukocyte are then perfused through the microfluidic channels at a defined flow rate, enabling the interaction between leukocytes and pre-coated adhesive substrates, which is digitally recorded by fluorescent video microscopy. The behaviours of interacting cells may be analysed utilizing a software that incorporates cell kinetic parameters and quantitatively characterises the leukocyte/ligand interaction kinetics

### Example 3. Workflow for Performing Image and Data Analysis for LAFA

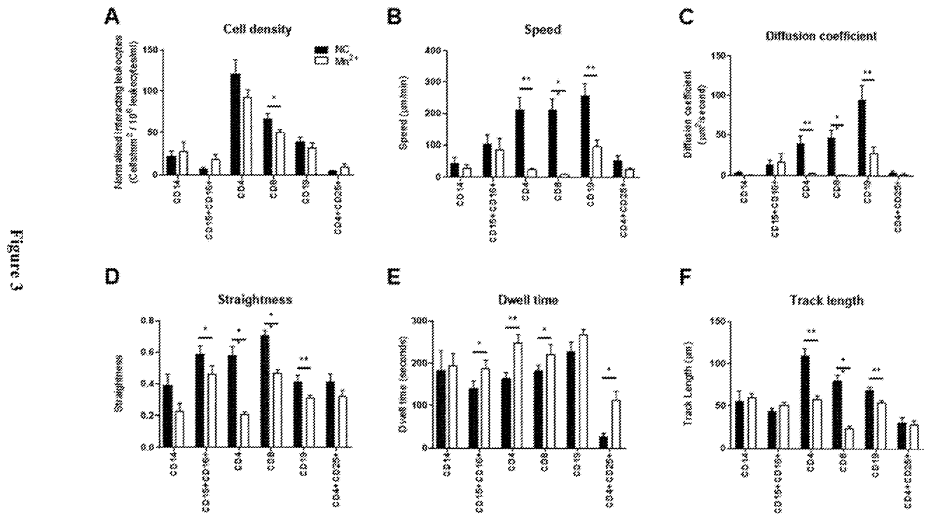

The present Example is directed to providing an example flow chart to perform image and data analysis for leukocyte adhesive function assay (LAFA). Images are recorded by a fluorescent microscope and may be then further analysed following the steps (FIG. 2).

Different analysis pipelines are in place depending on required output and speed. a) Conventional Analysis Pipeline (FIG. 2A)

Fiji image analysis software and R studio are used to process and analyse images generated during leukocyte adhesive function assay (LAFA), so that a range of cell kinetic parameters may be determined and used to characterise the cell migratory behaviours.

For example, the images and data analysis process may consist of the following steps:
1. Raw TIF images captured with the microscope are opened in Fiji image analysis software and reorganized into a time-lapse sequence.
2. Correct scaling information is applied. Flow channel edges are removed from images by cropping. An image flattening algorithm is applied to remove uneven background fluorescence.
3. Image sequence is split into individual channels for analysis.
4. TrackMate plugin from Fiji software is used to track individual cells with a set cell size and intensity threshold per channel.
5. The outputs from TrackMate are further analysed by R Statistical Software package to convert the data to the desired measuring units for a range of cell kinetic parameters, including but not limited to cell numbers, speed, straightness, dwell time, diffusion coefficient.
6. and to generate descriptive statistical graphs (e.g. box-and-whisker plots of kinetic parameters, speed distribution histogram, straightness distribution histogram, duration histogram, dwell time distribution histogram, motility curves, common origin graphs, appearance graphs).

In addition, other image software may be used to analyse the images and generate results.

Batch processing scrips are in place to automate image analysis in a set-and-forget approach; start the analysis with the push of a button and collect analysis results when the images have been processed and analysed.

b) Machine Learning trained on Images (FIG. 2B)

TensorFlow in Python was used to develop and train a machine learning algorithm on standard deviation projections of the raw image time-lapse sequence.

For example, the images and data analysis process may consist of the following steps:
1. Raw TIF images captured with the microscope are converted into standard deviation projections for each experiment.
2. Algorithm is trained on standard deviation projections of data with known disease state (‘basal’ or ‘abnormal’). Importantly, this step is only required the first time. After the algorithm has been trained, step 2 may be omitted and results (step 3) may be obtained immediately.
3. Algorithm predicts ‘basal’ or ‘abnormal’ status for unknown data sets.

c) Machine Learning trained on Tracking Results (FIG. 2C)

The RandomForest package in R was used to develop and train a machine learning algorithm on TrackMate tracking results.

For example, the images and data analysis process may consist of the following steps:
1. Raw TIF images captured are analysed with the Conventional Analysis Pipeline (a).
2. Algorithm is trained on tracking results (obtained in step 4a) of data with known disease state (‘basal’ or ‘abnormal’). Importantly, this step is only required the first time. After the algorithm has been trained, step 2 may be omitted and results (step 3) may be obtained immediately.

Algorithm predicts ‘basal’ or ‘abnormal’ status for unknown data sets.

### Example 4. Mn²⁺ Activates α4β1 Integrin Adhesive Functions on VCAM-1 Substrate

The present example is directed to testing the ability of leukocyte adhesive function assay (LAFA) to quantitatively assess the Mn²⁺-induced activation of leukocyte α4β1 integrin. Leukocytes in whole blood collected from healthy volunteers were treated with or without 5 mM MnCl₂, a pan integrin activator, before being used for LAFA using VCAM-1 as adhesive substrate. Unless otherwise stated the protocol used is set for in Methods and Materials.

The following criteria were used to define a healthy subject or healthy subjects:
1. Overtly healthy, as determined by medical evaluation including medical history
2. Women who are NOT pregnant or currently lactating
3. Not being diagnosed for autoimmune, inflammatory, hematologic and vascular disorders
4. Currently NOT taking prescribed medication, except for contraceptives
5. Currently NOT taking over-the-counter medications that may affect blood cell functions, including anti-histamine drugs, aspirin etc. Vitamin supplements are acceptable for this study
6. Currently do not have an upper respiratory tract infection (i.e. common cold), fever or known allergic reactions
7. No recent (last 5 years) smoking history

Mn²⁺, a pan integrin activator, activates α4β1 integrin on leukocyte membrane and, therefore leads to an increased α4β1 integrin binding activity to its endothelial ligand, VCAM-1. To test the ability of the system to detect Mn²⁺-induced α4 integrin activation, heprinised whole blood from healthy volunteers was treated with or without Mn²⁺ for approximately 5 minutes at room temperature (20° C.) before being used for LAFA on VCAM-1 substrate. To achieve concurrent detection of multiple specific leukocyte subsets, multi-colour fluorescently labelled antibodies against specific leukocyte membrane markers were added to human whole blood and incubated at room temperature for approximately 5 minutes, before performing the flow assay. The leukocytes were visualised by a fluorescent microscope and different subpopulations of leukocytes were distinguished, for example, based on the different wave length of their specific fluorescence.

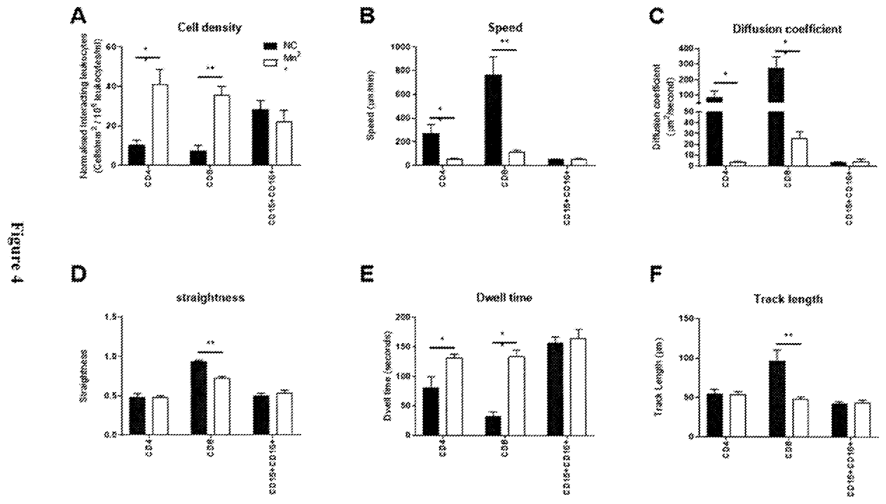

As indicated in FIG. 3A, comparing to untreated controls, Mn²⁺ did not significantly alter the number of interacting cells in the leukocyte subsets, except for a small decrease in CD8 cells (p<0.05), suggesting that cell density alone may not be enough to accurately assess Mn²⁺ effects on leukocyte adhesive function. On the other hand, the cell speed of CD4 (p<0.01), CD8 (p<0.01) and CD19 (p<0.01) was significantly reduced by the Mn²⁺ treatments, related to untreated controls. These findings show that Mn²⁺ enhances α4β1 integrin ligand binding ability and cell-VCAM-1 interaction, leading to a reduction of cell migration speed.

Diffusion coefficient, a cell kinetic parameter, is a measure for how fast cells displace from their start points during a random walk process, describing whether the cell motion is random (low diffusion coefficient value) or direct (high diffusion coefficient value) (Kucik, 1996; Beltman, 2009). As shown in FIG. 3C, compared to the controls, the diffusion coefficient values of CD4, CD8, CD19 and CD4+CD25+ cells were significantly reduced by Mn²⁺ treatments, showing a suppressive effect of Mn²⁺ on cell migration. Consistently, the cell straightness (defined as the ration between cell displacement and cell track length) of the leukocyte subsets except for CD14 and CD4+CD25+ cells was significantly decreased in the presence of Mn²⁺ (FIG. 3D). These findings indicated that Mn²⁺ treatments activated α4β1 integrin adhesive function, leading to a stronger cell interaction with VCAM-1.

In addition, the dwell time of CD15+CD16+, CD4, CD8 and CD4+CD25+ cells was increased by Mn²⁺ (FIG. 3E), supporting the notion that Mn²⁺ activates leukocyte α4β1 integrin, resulting in stronger cell binding to VCAM-1 substrate and then a suppression of cell motion. Consistently, the track length of CD4, CD8 and CD19 cell was reduced by Mn²⁺, related to untreated controls (FIG. 3F), suggesting an inhibited cell mobility. The fact that no effects of Mn²⁺ treatment on CD14 cells was detected in the cell kinetic parameters (FIGS. 3A-F) suggests a unique role of α4β1 integrin in the regulation of CD14 cell recruitment.

Together, these results demonstrated, for example, the ability of LAFA to quantitatively assess Mn-induced α4β1 integrin activation in a range of leukocyte subsets. The ability of LAFA to detect Mn²⁺ specific effects on specific cell kinetic parameters in different leukocyte sub-populations demonstrates a good sensitivity and specificity of LAFA. LAFA (VCAM-1) may then be used to assess the adhesive function of α4β1 integrin in Examples 6 to 11.

### Example 5. Mn²⁺ Activates α4β7 Integrin Adhesive Functions on MAdCAM-1 Substrate

The present example is directed to testing the ability of leukocyte adhesive function assay (LAFA) to quantitatively assess the Mn²⁺-induced activation of leukocyte α4β7 integrin. Leukocytes in whole blood collected from healthy volunteers were treated with or without 5 mM MnCl₂, a pan integrin activator, before being used for LAFA using MAdCAM-1 as adhesive substrate. Unless otherwise stated the protocol used is as outlined in the Methods and Materials.

To characterise the effect of Mn²⁺ on α4β7 integrin adhesive function, a range of cell kinetic parameters were utilised to determine cell migratory behaviours on MAdCAM-1 substrate. As shown in FIG. 4A, comparing to untreated controls, Mn²⁺ treatments significantly increased the number of CD4 and CD8 cells. These results suggested that Mn²⁺ activated α4β7 integrin on CD4 and CD8 cells, leading to an increased cell binding to MAdCAM-1 substrate, in line with known action of Mn²⁺ on α4β7 integrin.

Similarly, related to controls, the cell speed and diffusion coefficient of CD4 and CD8 cell were both significantly reduced, whereas the dwell time was significantly increased (FIGS. 4B, 4C and 4E) by Mn²⁺. These findings support notion that Mn²⁺ enhances α4β7 integrin adhesive function and cell-MAdCAM-1 binding, resulting in suppression of cell motion. It was also noted that the straightness and track length of CD8 cells only (not CD4 cells) was decreased by Mn²⁺ treatments, indicating that the role of α4β7 integrin in regulation of cell migration may differ between CD4 and CD8 cells (FIGS. 4D and 4F). The fact that no Mn²⁺ effect on CD15+CD16+ cell (neutrophil) migratory behaviour was detected (FIGS. 4A-4F), suggesting a divergent role of α4β7 integrin in the regulation of neutrophil recruitment, comparing to CD4 and CD8 cells. Together, these results demonstrated, for example, the ability of LAFA to quantitatively assess Mn-induced α4β7 integrin activation in several different leukocyte subsets. LAFA (VCAM-1) was then be used to assess the adhesive function of α4β1 integrin, as described in some of the following Examples.

### Example 6. Assessment of Leukocyte Adhesion Function in SIRS Patients by LAFA Using VCAM-1 as a Substrate

The present Example is directed to testing the ability of leukocyte adhesive function assay (LAFA) to detect an elevated immune response in patients with systematic inflammatory response syndrome (SIRS). Blood samples were collected from healthy volunteers and SIRS patients, and then analysed by LAFA on VCAM-1 substrate. Unless otherwise stated the protocol used is set for in Methods and Materials.

Fourteen SIRS patients were recruited for this study by screening patients who were newly admitted to Intensive Care Unit (ICU). Patients were qualified to the study if >2 of the following four criteria were met, regardless of the causes of inflammation:
1. Body temperature >38° C. or <36° C.
2. Heart rate >90 per minute
3. Respiration rate >20 breaths per minute or PaCO₂<32 mmHg
4. White blood cell count >12,000/mm³ or <4,000/mm³ or >10% bands.

The blood samples were collected with 48 hours after the first identification of the systemic inflammatory response.

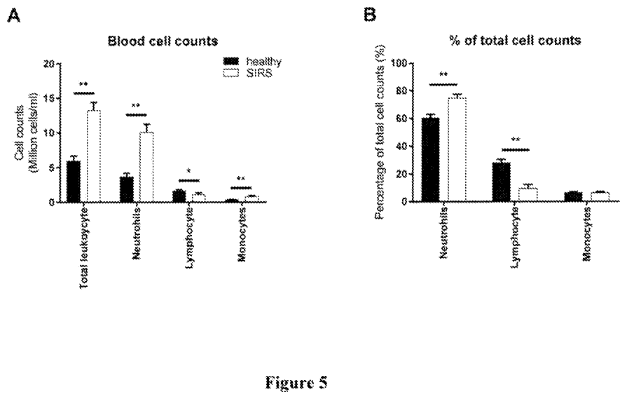

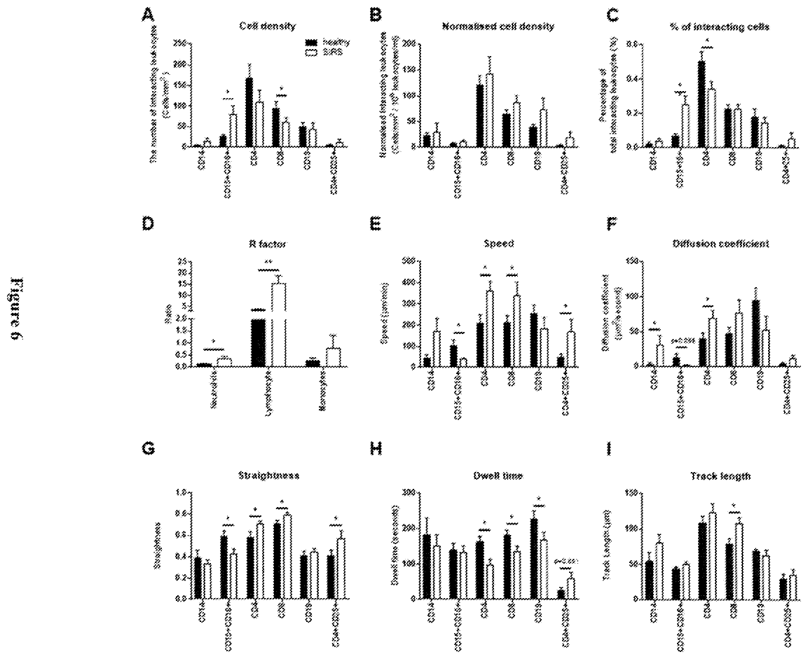

A full blood cell count was performed for the blood samples, using a Mindray BC5000 Haematology Analyser according to manufacturer's instructions. As shown in FIG. 5A, compared with healthy controls, an elevated total leukocyte count was observed in SIRS patients, which is mainly due to an elevated neutrophil count. Consistently, the percentage of neutrophil was also higher in SIRS patient than healthy controls (FIG. 5B). In addition, the cell count and percentage of lymphocytes were significantly lower in SIRS patients, related to healthy controls (FIGS. 5A and 5B). On the other hand, a significantly higher monocyte count was detected in SIRS patients (FIG. 5A), whereas no difference was observed in the percentage of monocytes between SIRS patients and healthy controls (FIG. 5B).

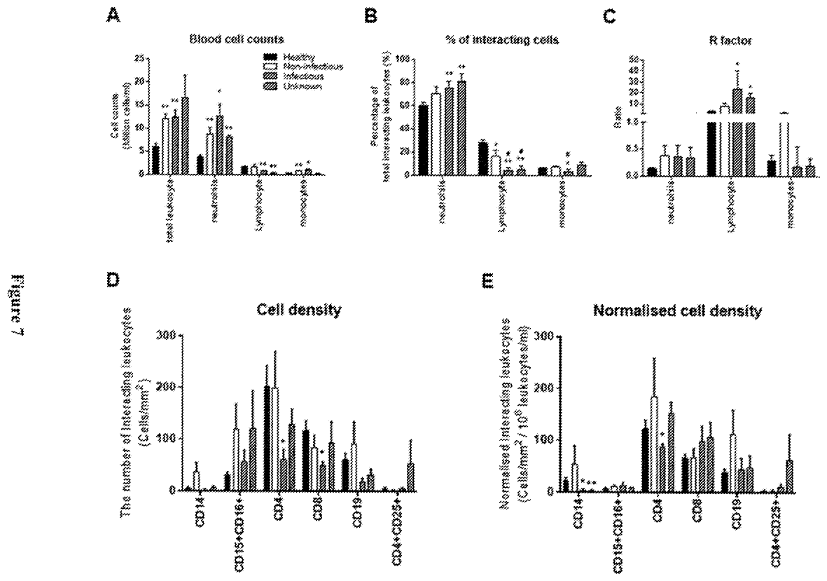

The blood samples were analysed by LAFA on VCAM-1 substrate. As shown in FIG. 6A, compared with healthy controls, the cell density of CD15+CD16+ neutrophil was significantly higher in SIRS patients, whereas a slight decrease of interacting CD8 cells was observed in SIRP patients. When the cell density was normalised using appropriate leukocyte counts, however, no difference was detected in the cell types between control and SIRP patients (FIG. 6B).

The percentage of specific leukocytes in total interacting cells was also determined. It was found that the percentage interacting neutrophils was significantly higher in SIR patients, while the percentage of CD4 cell was lower, compared with healthy controls (FIG. 6C). In addition, a recruitment factor (R factor), an indicator for the propensity of a specific leukocyte population to be recruited, is calculated as (% of cell type)/(% cell type in circulation) (Ibbotson, 2001). As indicated in FIG. 6D, the R factor values of neutrophils (CD15+CD16+) and lymphocytes (a sum of CD4, CD8 and CD19 cells) are both significantly higher in SIRS patients, related to healthy controls. These findings demonstrate, for example, an enhanced neutrophil α4β1 integrin adhesive function in SIRS patients, leading to an elevated neutrophil ability to interact with VCAM-1 substrate.

Next, a range of cell kinetic parameters were used to determine the elevated cell adhesive function in SIRS patients. As shown in FIG. 6E, the speed of CD15+CD16+ cell (neutrophils) was significantly lower in SIRS patients, related to healthy controls, suggesting an enhanced α4β1 integrin activity on SIRS neutrophils. This notion was further supported by the fact that the diffusion coefficient and straightness of neutrophils were decreased in SIRS patients (FIGS. 6F and 6G), indicate an inhibition of cell mobility. These results suggest that α4β1 integrin adhesive function is abnormally elevated on CD15+CD16+ cells from SIRS patients, which may be used a marker to determine an elevated immune response in these patients.

On the other hand, the speed of CD4, CD8 and CD4+CD25+ cells were higher in SIRS patients, suggesting a reduced adhesive function of α4β1 integrin in SIRS patient, compared with health controls (FIG. 6E). Consistently, the diffusion coefficient and straightness of CD4 and CD8 cells were found to be higher, whereas the dwell time was shorter in SIRS patient than healthy controls, suggesting a low activity of α4β1 integrin in SIRS patients (FIGS. 6F, 6G and 6H). These findings indicate that α4β1 integrin on CD4 and CD8 cells may play a different role in the regulation of the enhanced inflammatory response in SIRS patients.

Together, these results demonstrate the ability of LAFA to generate a range of cell kinetic markers, which may be used to identify α4β1 integrin activation in SIRS patients.

### Example 7. Distinguishing Non-Infectious SIRS from Infectious SIRS by LAFA Using VCAM-1 as a Substrate

The present example is directed to test the ability of leukocyte adhesive function assay (LAFA) to distinguish non-infectious SIRS from infectious SIRS and/or healthy subjects using VCAM-1 as a substrate. Based on their clinical records (detailed in Example 8), each of the 14 SIRS patients (the same cohort as in Example 6) were retrospectively assessed to determine if it represents either:
1. Group “Non-infectious”: Infection UNLIKELY or
2. Group “Infectious”: PROVEN infection (e.g. Positive microbiology result) or
3. Group “Unknown”: Infection possible BUT not proven.

Thus, the different α4β1 integrin activity in these 3 groups was then determined by LAFA on VCAM-1 substrate.

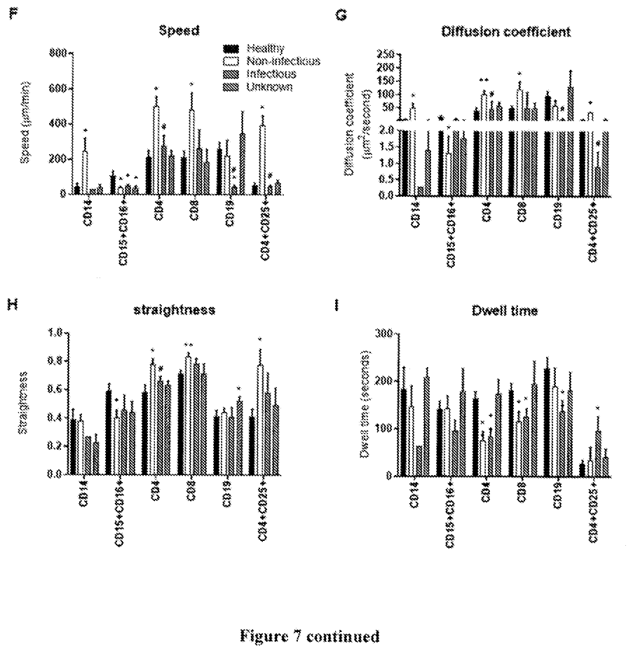

As shown in FIG. 7A, the total leukocyte counts were significantly higher in the three groups of SIRS patients, compared with healthy controls, mainly due to elevated neutrophil counts. The lymphocyte counts were lower in infections and unknown group, related to healthy controls, whereas no difference was found between non-infectious and healthy groups (FIG. 7A). On the other hand, monocyte counts were higher in non-infectious and infectious groups but not in unknown group than healthy controls (FIG. 7A).

After being analysed by LAFA, the total number of interacting cells were determined for multiple leukocyte sub-populations. As shown in FIG. 7B, the percentage of interacting neutrophils was significantly higher in infectious and unknown groups than healthy controls, but not in non-infectious group, suggesting that neutrophil α4β1 integrin was specifically activated in infectious SIRS patients. These results are in line with the previous finding showing an abnormally enhanced neutrophil recruitment by VCAM-1 in sepsis patients (Ibbotson, 2001), suggesting that α4β1 integrin activation on neutrophils may be used a marker to distinguish infections SIRS from non-infectious SIRS.

It was also noted that the percentage of interacting lymphocytes was significantly decreased in the three SIRS groups, compared with healthy controls (FIG. 7B). The lymphocyte percentage was also significantly lower in the infectious group than non-infectious group, which may also be used as a marker to separate infectious SIRS and non-infectious SIRS. Similarly, the cell density of CD4 cells, with or without being normalised by the lymphocyte counts, are both lower in infectious SIRS than healthy controls (FIGS. 7D and 7E).

In addition, the percentage of interacting monocytes was lower in infectious group than heathy and non-infectious groups (FIG. 7B). Similarly, the normalised CD14 cell density was found to be lower in infectious group, compared with healthy controls (FIG. 7E). These findings suggest a reduced VCAM-1 induced monocyte recruitment may also be used a mark to distinguish non-infectious SIRS from infectious SIRS.

Multiple cell kinetic parameters were also employed to determine different α4β1 integrin adhesive function between three SIRS groups. As shown in FIG. 7F, the cell speed of CD14, CD4, CD8 and CD4+CD25+ cells was significantly greater in non-infectious SIRS patients than healthy controls, and no such increase was observed in infectious SIRS group. The speed of CD4 and CD4+CD25+ cells was also significantly lower in infectious SIRS group than non-infectious SIRS group (FIG. 7F).

Consistently, the diffusion coefficient of CD14, CD4, CD8 and CD4+CD25+ cells was found to be higher in infectious SIRS group, but not in non-infectious group, compared with healthy controls (FIG. 7G). A significantly lower diffusion coefficient of CD4, CD19 and CD4+CD25+ cells was also observed in infectious SIRS group, related to non-infectious SIRS group (FIG. 7G). Similarly, the straightness of CD4, CD8 and CD4+CD25+ cells was significantly higher in non-infectious SIRS patients than healthy controls, but no such difference was seen infectious SIRS group (FIG. 7H).

In addition, the speed of CD19 cell was lower in infectious SIRS group than non-infectious SIRS group (FIG. 7F). Similarly, the diffusion coefficient and dwell time of CD19 cell was lower in infectious SIRS group than healthy controls, but no such difference was detected in non-infectious SIRS (FIGS. 7G and 71).

Together, these findings demonstrate the ability of LAFA to generate a range of new markers to assess α4β1 integrin adhesive in three SIRS groups. LAFA has identified a number of useful cell kinetic markers that may be used to distinguish non-infectious SIRS from infectious SIRS patients.

### Example 8. Use Single Cell Profiles to Distinguish Specific Cell Adhesion Function in Individual SIRS Patients by LAFA on VCAM-1 Substrate

The present Example is directed to the use of single cell profile generated by leukocyte adhesive function assay (LAFA) to determine specific cell adhesive function in individual SIRS patients. Blood samples were collected from healthy volunteers and SIRS patients, and then analysed by LAFA on VCAM-1 substrate. Unless otherwise stated the protocol used is set for in Methods and Materials.

For each blood samples collected from SIRS patients, standard microbiological testing was performed to determine potential positivity for infections. In combination of clinical records, potential causes of inflammation in individual SIRS patients was then determined by two experienced ICU specialists independently. As a result, the potential causes of systemic immune response in each SIRS patient are listed in Table 3. Accordingly, the 14 SIRS patients were divided into three groups: 1) Non-infectious, 2) Infectious and 3) Unknown, as detailed in Example 7.

TABLE 3
The causes of systemic inflammatory response in individual
SIRS patients. Blood samples were collected from each
SIRS patients and standard microbiological testing was
performed to determine potential infection. In combination
of clinical records, the causes of inflammation in individual
SIRS patients were then determined.

| Groups | Codes | Causes of inflammation |
|---|---|---|
| Healthy | H-01 | Healthy #01 |
| | H-02 | Healthy #02 |
| | H-03 | Healthy #03 |
| Non-infectious SIRS | SIRS-01 | Cardiac arrest #01 |
| | SIRS-02 | Cardiac arrest #02 |
| | SIRS-03 | Asthma |
| | SIRS-04 | Post-surgery |
| | SIRS-05 | Subarachnoid haemorrhage |
| | SIRS-06 | Hepatic art aneurysm |
| Infectious SIRS | SIRS-07 | Enteric bacteria and Pseudomonas |
| | SIRS-08 | E. coli |
| | SIRS-09 | Staphylococcus aureus |
| | SIRS-10 | Serratia, enterococcus, pseudomonas |
| | SIRS-11 | Streptococcus group G |
| Unknown | SIRS-12 | Post BEAM Autograft |
| | SIRS-13 | Possible asthma |
| | SIRS-14 | Aspiration pneumonitis |

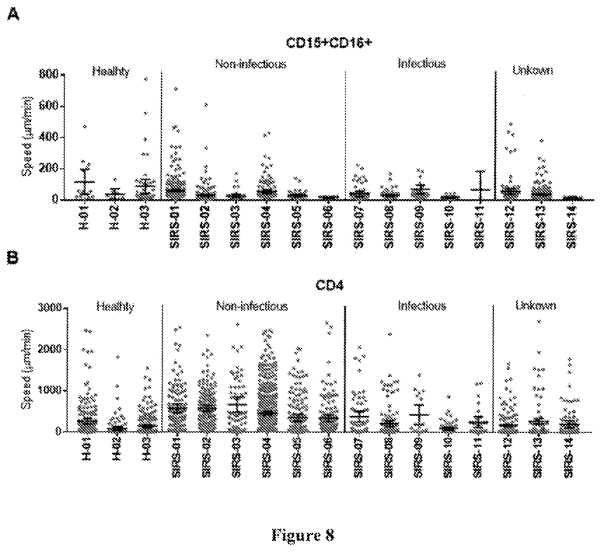

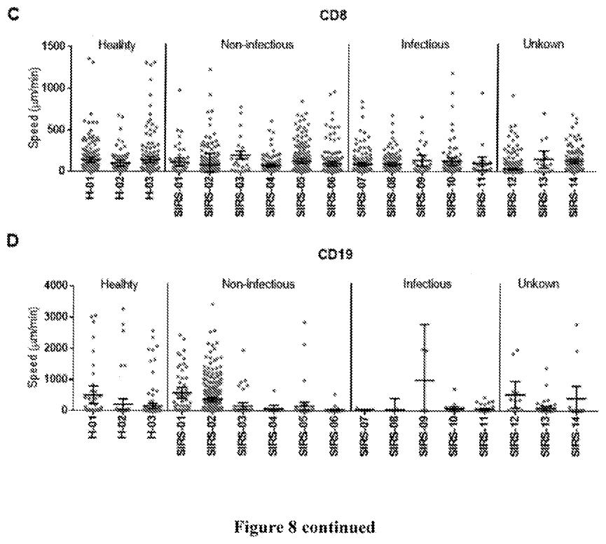

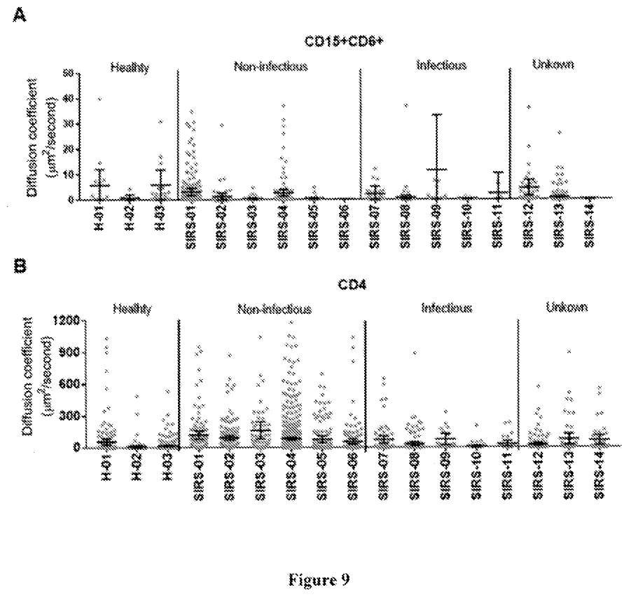

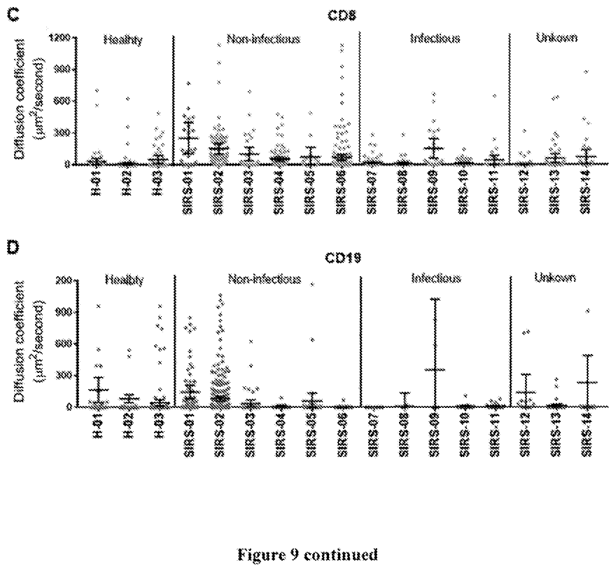

Each of the blood samples was analysed by LAFA on VCAM-1 substrate, and several cell kinetic parameters of each single interacting cell were then determined. As shown in FIG. 8A, the number of CD15+CD16+ cells are higher in all SIRS patients than the three healthy subjects, except for patient SIRS-11 whose inflammation was caused by Streptococcus group G infection. In addition, a high cell speed and diffusion coefficient of CD4 cells were seen in non-infectious SIRS patients (FIGS. 8B and 9B), while it was low in three healthy subjects, in line with the data presented in FIGS. 6E and 6F.

It is also noted that only a few CD19 interacting cells were detected in patient SIRS-04 (post-surgery) (FIG. 8D), while the CD4 cell density of is very high (FIG. 8B). In addition, the speed and diffusion coefficient of a large portion of these CD4 cells are both high, compared with other subjects (FIGS. 8B and 9B), suggesting an unique cell immune activation in this patient.

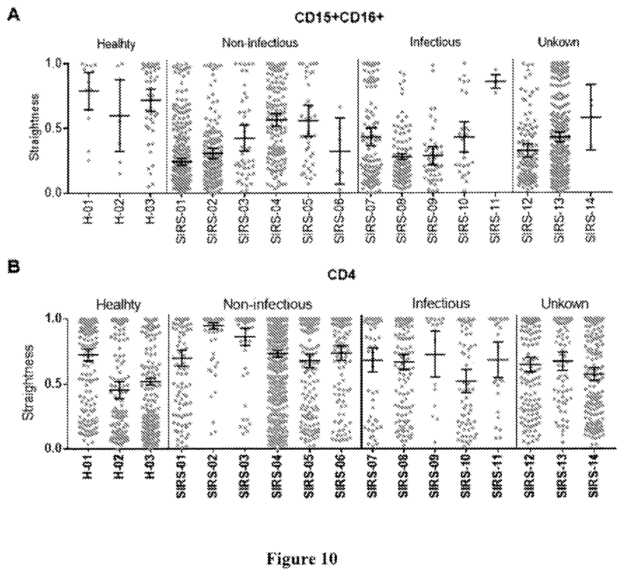

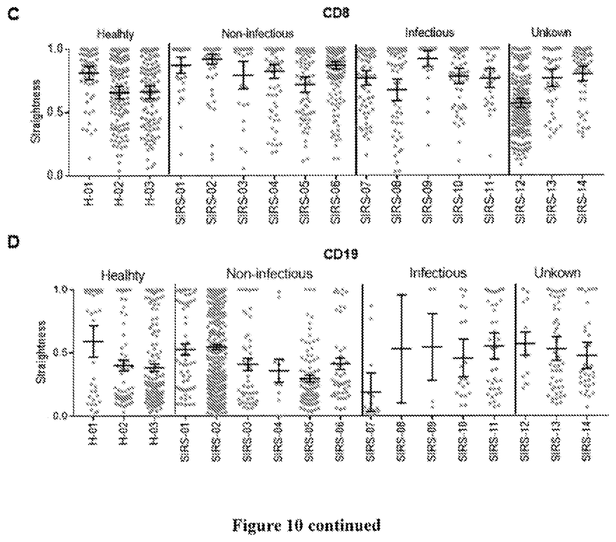

Interestingly, the CD19 cell density was consistently elevated in two SIRS patients with cardiac rest (SIRS-01 and SIRS-02), related to other non-infectious SIRS patients or healthy subjects, suggesting a specific CD19 response in these two SIRS patients (FIG. 8D). Similarly, the straightness of CD15+CD16+ cells of these two cardiac arrest patients was low, whereas these patients' CD19 cell straightness was high related to other non-infectious SIRS patients, suggesting similar cell immune status in patients with cardiac arrest (FIGS. 10A and 10D), which may be used to distinguish cardiac arrest-induced SIRS from other causes.

Together, these results show the ability of single cell profile to generated useful markers to assess cell adhesive function in individual patients. The results disclosed herein suggest that the single cell profiles may also be used to distinguish specific causes of inflammation in individual SIRS patients.

### Example 9. Assess Leukocyte Adhesion Function in SIRS Patients by LAFA Using VCAM-1 Plus IL-8 as Substrates

The present example is directed to test the ability of leukocyte adhesive function assay (LAFA) to detect an elevated immune response in patients with systematic inflammatory response syndrome (SIRS), using VCAM-1 plus IL-8 as substrates. The ability of LAFA (VCAM-1+IL-8) to distinguish non-infectious SIRS from infectious SIRS was also determined. Blood samples were collected from healthy volunteers and SIRS patients, and then analysed by LAFA on VCAM-1 plus IL-8 substrates. Unless otherwise stated the protocol used is set for in Methods and Materials.

IL-8 is a chemokine that may guide the migration of leukocytes by forming a concentration gradient, and IL-8 is shown to mainly induce neutrophil chemotaxis. CXCR1, receptors for IL-8, may be expressed on leukocyte membranes, and plays a role in the regulation of leukocyte functions and migratory behaviours. In this example, in combination with VCAM-1, IL-8 was used as adhesive substrates, so that the role of CXCR1 in regulation of leukocyte adhesive function in SIRS patients may be assessed using a range of cell kinetic parameters.

As shown in FIG. 11A, an increased number of interacting CD14, CD15+CD16+ and CD4+CD25+ cells was detected in SIRS patients, compared with healthy controls. Once being normalised with appropriate leukocyte cell counts, however, only an increase in CD4+CD25+ cell density in SIRS patients was observed (FIG. 11B).

The percentage of interacting CD4+CD25+ regulatory cells was significantly higher in SIRS patients than healthy controls (FIG. 11C), which was not found when using VCAM-1 alone as substrate (FIG. 6C), suggesting a functional role of CXCR1 in the regulation of CD4+CD25+ cell adhesive function.

As previously demonstrated in FIG. 6 in Example 6, the adhesive functions of CD15+CD16+ neutrophils was enhanced in SIRS patients, evidenced by an decrease of cell speed, diffusion coefficient and straightness. On VCAM-1 plus IL-8 substrate, however, no such difference in cell speed and diffusion coefficient was detected between healthy and SIRS patients (FIGS. 11E and 11F). These findings suggest that neutrophil CXCR1 may receive signals from IL-8 substrate, which may have direct inhibitory effects on cell mobility in both healthy and SIRS cells. As a result, the difference of neutrophil mobility between healthy and SIRS subjects was concealed in the presence of IL-8.

In addition, the speed, diffusion coefficient, dwell time and track length of CD8 interacting cells were found to be significantly higher in SIRS patients than healthy controls (FIGS. 11E, 11F, 11H and 11I). These results show different cell migratory behaviours between healthy and SIRS CD8 cells, which may be used to assess abnormal immune response in SIRS patients.

As detailed in Example 7, based on their clinical records, the 14 SIRS patients were divided into three groups: 1) Non-infectious, 2) Infectious and 3) Unknown. Thus, the ability of LAFA (VCAM-1+IL-8) to distinguish non-infectious SIRS from infectious SIRS was tested.

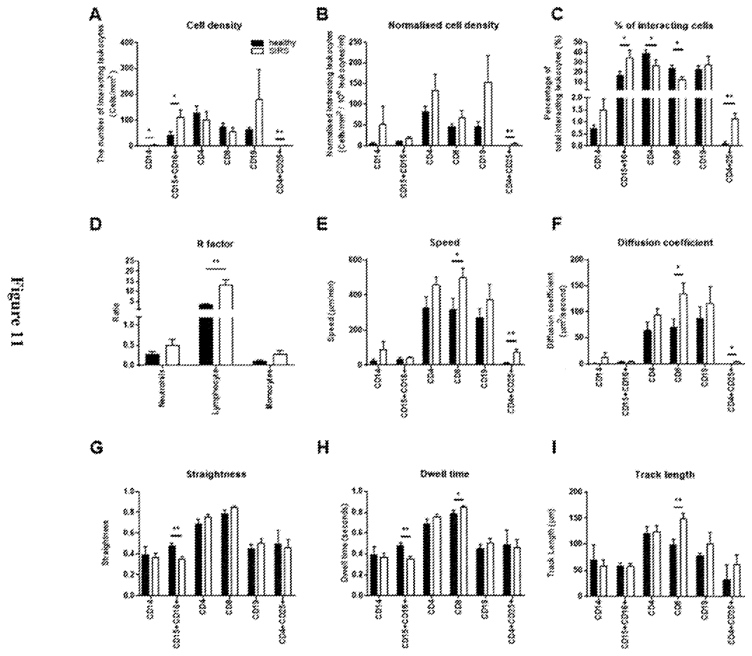

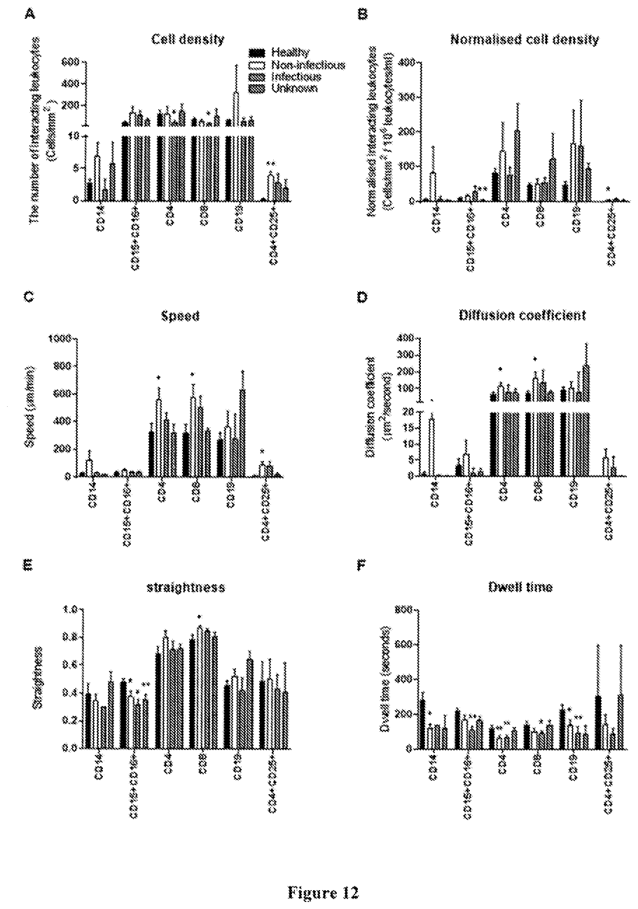

As shown in FIG. 12A, the number of interacting CD4 and CD8 cells was significantly lower in infectious SIRS group than healthy controls, whereas no such decrease was detected in non-infectious SIRS. In addition, compared with healthy controls, the dwell time of CD15+CD16+ neutrophils was lower in infectious SIRS group but not in non-infectious SIRS group. These results suggest that these new markers may be used to separate non-infectious SIRS from infectious SIRS.

Together, these results show a functional role of CXCR1 in regulation of leukocyte adhesive function in healthy and SIRS leukocytes. A range of new markers were generated by LAFA using VCAM-1 plus IL-8 substrates, which may be used to determine divergent leukocyte CXCR1 activity on healthy and SIRS leukocytes. Additionally, the results disclosed herein indicate that a number of new LAFA markers may be used to distinguish non-infectious SIRS from infections SIRS.

### Example 10. Assess Leukocyte Adhesion Function in SIRS Patients by LAFA Using VCAM-1 Plus SDF-La as Substrates

The present Example is directed to testing the ability of leukocyte adhesive function assay (LAFA) to detect an elevated immune response in patients with systematic inflammatory response syndrome (SIRS), using VCAM-1 plus SDF-1α as substrates. The ability of LAFA (VCAM-1+SDF-1α) to distinguish non-infectious SIRS from infectious SIRS was also determined. Blood samples were collected from healthy volunteers and SIRS patients, and then analysed by LAFA on VCAM-1 plus SDF-1α substrates. Unless otherwise stated the protocol used is set for in Methods and Materials.

SDF-1α, also known as CXCL12, is a chemokine that may guide the migration of leukocytes by forming a concentration gradient. SDF-1α is shown to mainly induce lymphocyte chemotaxis. CXCR4, receptors for SDF-1α, may be expressed on leukocyte membranes, and plays a role in the regulation of leukocyte functions and migratory behaviours. In this example, in combination with VCAM-1, SDF-1α was used as adhesive substrates, so that the role of CXCR4 in regulation of leukocyte adhesive function in SIRS patients may be assessed using a range of cell kinetic parameters.

As shown in FIGS. 13A and 13D, the cell density and R factor of CD14 monocytes is significantly higher in SIRS patients than healthy subjects. In addition, compared with healthy subjects, the straightness of CD14 cells was significantly lower in SIRS patients (FIG. 13D), whereas no such difference was observed in the absence of SDF-1α (FIG. 6G), suggesting an elevated activity of CXCR4 on SIRS CD14 cells than healthy cells.

It is also noted that >50% of the total interacting cells are neutrophils in SIRS patients, whereas the most dominant sub-population in healthy subjects is CD4 cells (>30%), suggesting an enhanced CXCR4 activity in SIRS neutrophils (FIG. 13C). Consistently, the cell speed of SIRS neutrophils is significantly higher (FIG. 13E), while the straightness and dwell time of SIRS neutrophils is lowered than healthy subjects (FIGS. 13G and 13H), suggesting an elevated responsiveness of SIRS neutrophils to SDF-1α, leading to an increased cell chemotaxis.

As detailed in Example 7, based on their clinical records, the 14 SIRS patients were divided into three groups: 1) Non-infectious, 2) Infectious and 3) Unknown. Thus, the ability of LAFA (VCAM-1+SDF-1α) to distinguish non-infectious SIRS from infectious SIRS was tested.

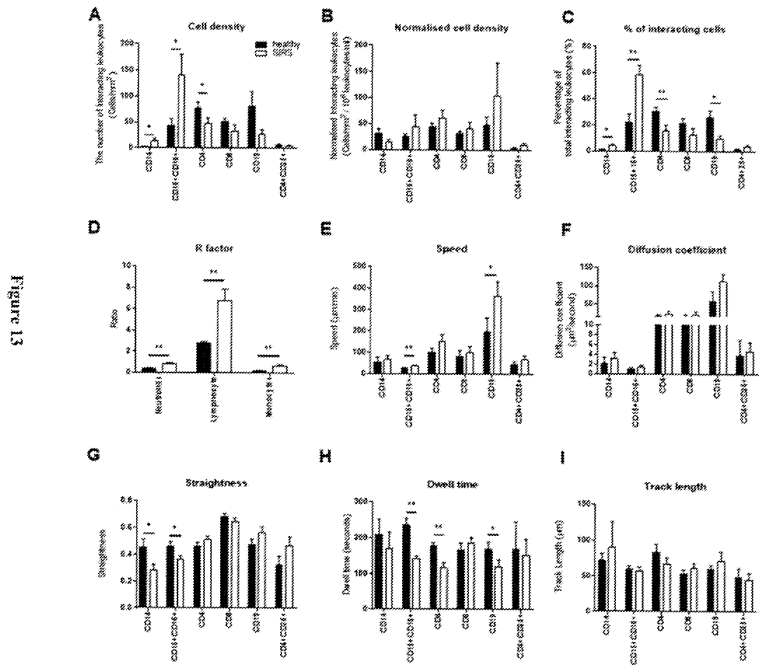

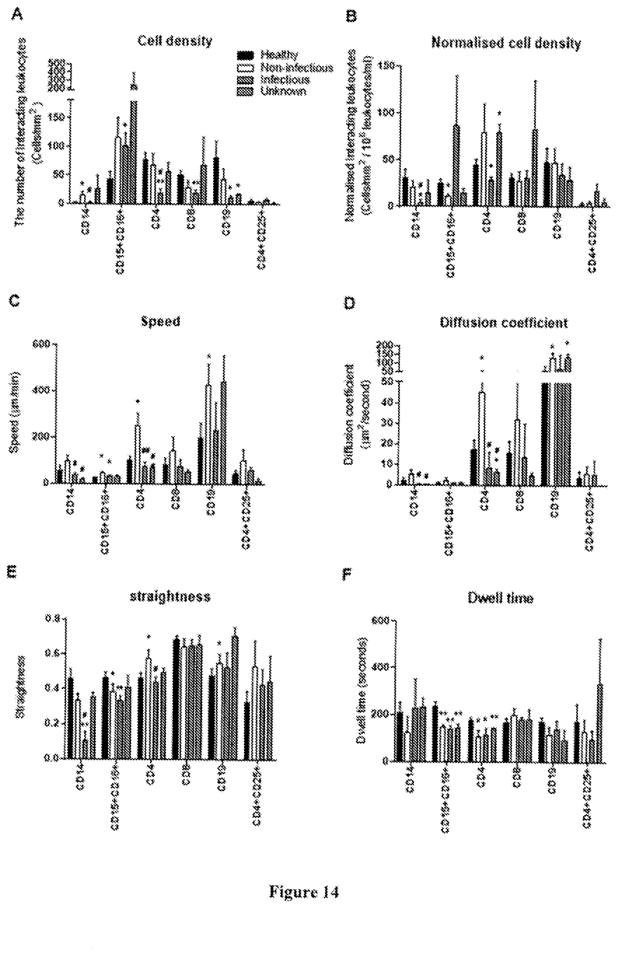

As shown in FIGS. 14A and 14B, the cell density (with or without normalization) of CD14 cells is significantly lower in infectious SIRS group than non-infectious SIRS group. On the other hand, the cell speed, diffusion coefficient and straightness of CD14 cells are significantly lower in infectious SIRS group than non-infectious SIRS group (FIGS. 14C, 14D and 14E). These results suggest that, despite a low cell recruitment, the mobility of interacting CD14 cells from infectious SIRS patients was lower than non-infectious SIRS patients, suggesting these markers may be used to separate non-infectious SIRS patients from infectious SIRS patients. Similarly, a lower CD4 cell density in infectious SIRS patients was also observed, compared with non-infectious SIRS group (FIG. 14A). It is also noted that the speed, diffusion coefficient and straightness of CD4 cells are lower in infectious SIRS patients, related to non-infectious SIRS (FIGS. 14C, 14D and 14E).

Together, these results show that a range of new markers were generated by LAFA using VCAM-1 plus SDF-1α substrates, which may be used to determine an elevated CXCR4 activity in SIRS patients. Additionally, the results disclosed herein indicate that a number of new LAFA markers may be used to distinguish non-infectious SIRS from infections SIRS.

### Example 11. Assess the Effects of Mn2+ on Leukocyte Adhesion Function in SIRS Patients Using LAFA on VCAM-1 Substrate

The present example is directed to use leukocyte adhesive function assay (LAFA) to assess Mn²⁺ effects on leukocyte adhesive function in SIRS patients on VCAM-1 substrate. The ability of LAFA to distinguish non-infectious SIRS from infectious SIRS in the presence of Mn²⁺ was also determined. Blood samples were collected from healthy volunteers and SIRS patients and treated with 5 mM of MnCl₂ for 5 min at room temperature, before being analysed by LAFA on VCAM-1 substrate. Unless otherwise stated the protocol used is set for in Methods and Materials.

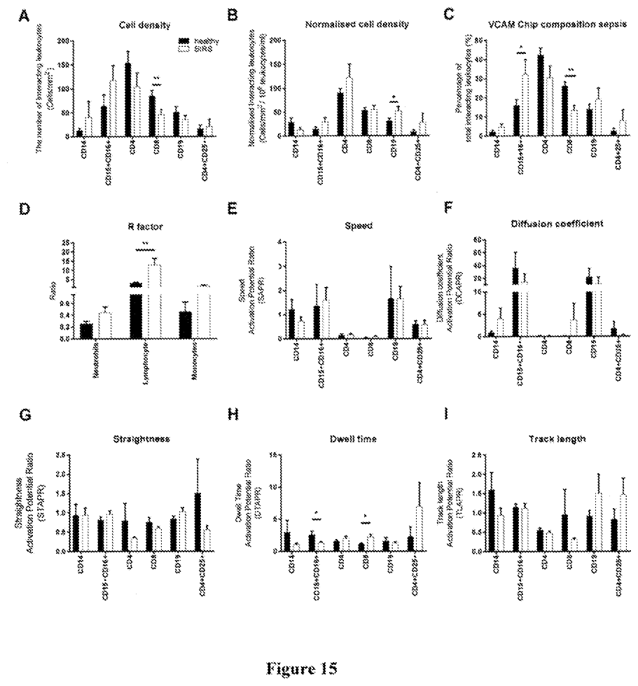

Mn²⁺, a pan integrin activator, activates α4β1 integrin on leukocyte membrane and, therefore leads to an increased α4β1 integrin binding activity to its endothelial ligand, VCAM-1. A shown in FIGS. 15A and 15C, the density and the percentage of interacting CD8 cells was both lower in SIRS patients than in healthy controls, suggesting a divergent activity of α4β1 integrin on CD8 cells between healthy and SIRS subjects in the presence of Mn²⁺.

To further characterise α4β1 integrin status, the difference of leukocyte ability to bind to VCAM-1 in the presence and absence of Mn²⁺ were used to generate “activation potential” of α4β1 integrin, showing how much α4β1 integrin activity may be induced by Mn²⁺. If cell α4β1 integrin is highly activated, the portion of activated α4β1 integrin may be high, which may indicate a low Mn²⁺ activation potential. Vice versus, a low cell α4β1 integrin activity may indicate a high Mn²⁺ activation potential. To assess the Mn²⁺ activation potential, activation potential ratio (APR) is introduced for a range of cell kinetic parameters. For example, if the average cell speed is Speed_{NC} and Speed_{Mn} (μm/min) in the absence and presence of Mn²⁺ respectively, the value of Speed Activation Potential Ratio (SAPR) is Speed_{Mn}/Speed_{NC}. In this case, the higher the SAPR value is, the less is the activation potential, meaning the higher portion of α4β1 integrin is in an activated form on the resting cells.

A Diffusion Coefficient Activation Potential Ratio (DCAPR) may be defined as the ratio between Diffusion Coefficient_{Mn} and Diffusion Coefficient_{NC} (Diffusion Coefficient_{Mn}/Diffusion Coefficient_{NC}). The higher the DCAPR value is, the less is the activation potential. The same formula (Straightness_{Mn}/Straightness_{NC}) may be used to determine Straightness Activation Potential Ratio (STAPR). The higher the STAPR value is, the less is the activation potential. Similarly, a Track Length Activation Potential Ratio (TLAPR) is a ratio of Track Length_{Mn}/Track Length_{NC}, and the higher TLAPR value is, the less is the activation potential.

Additionally, Dwell Time Activation Potential Ratio (DTAPR) may be defined as the ratio of Dwell Time_{Mn}/Dwell Time_{NC}. In this case, on the other hand, the higher the DTAPR value is, the higher is the activation potential.

Thus, SAPR, DCAPR, STAPR, TLAPR and DTAPR may be used to determine the portion of activated α4β1 and/or α4β7 integrins on a specific leukocyte population or populations, offering a semi-quantitative tool to assess the active status of leukocyte α4β1 and/or α4β7 integrins. Next, SAPR, DCAPR, STAPR TLARP and DTAPR may be used to assess the status of α4β1 integrins in healthy and SIRS subjects.

As shown in FIG. 15H, compared with healthy controls, the DTAPR value is significantly lower in CD15+CD16+ neutrophils of SIRS patients, suggesting a lower Mn²⁺ activation potential in SIRS neutrophils. On the other hand, the DTAPR is higher in SIRS CD8 cells than healthy subjects, suggesting a greater activation potential in SIRS CD8 cells.

As detailed in Example 7, based on their clinical records, the 14 SIRS patients were divided into three groups: 1) Non-infectious, 2) Infectious and 3) Unknown. Thus, the ability of SAPR, DCAPR, STAPR TLARP and DTAPR to distinguish non-infectious SIRS from infectious SIRS was tested.

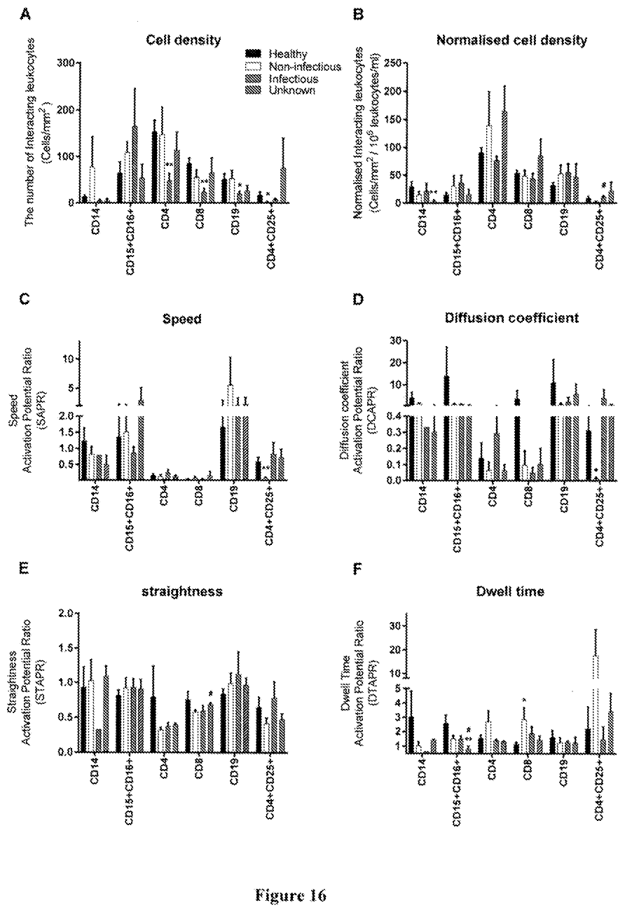

As shown in FIG. 16A, compared to healthy subjects, the cell density of CD4, CD8 and CD19 cells were significantly lower in infectious SIRS patients, while no such decrease was observed in non-infectious SIRS patients. A significantly greater DTARP value was detected in CD8 cells from non-infectious patients (but not in infectious group), related to healthy controls (FIG. 16F), suggesting a higher Mn²⁺ activation potential in non-infections CD8 cells.

Additionally, cell density of CD4+CD25+ cell was significantly higher in infectious SIRS group, compared with no-infectious SIRS group (FIG. 16B). The DCAPR value of CD4+CD25+ cells in non-infectious SIRS patients is significantly lower than healthy controls, while no such difference was observed in infectious SIRS patients. (FIG. 16D). These results suggest that these markers may be used to separate non-infectious SIRS from infectious SIRS.

Together, these results demonstrate the ability of LAFA to detect different activity status of α4β1 integrin in healthy and SIRS subjects. The results disclosed herein also show that SAPR, DCAPR, STAPR TLARP and DTAPR may be used as markers to distinguish non-infectious SIRS from infections SIRS.

### Example 12. Assess Leukocyte Adhesion Function in SIRS Patients by LAFA Using P-Selectin Plus E-Selectin as Substrates

The present Example is directed to testing the ability of leukocyte adhesive function assay (LAFA) to detect an elevated immune response in patients with systematic inflammatory response syndrome (SIRS) using P-selectin plus E-selectin as substrates. Blood samples were collected from healthy and SIRS subjects, and then analysed by LAFA on P-selectin plus E-selectin (ligands for leukocyte expressing PSGL-1) as substrates. The microfluidic channels were pre-coated with a combination of human P-selectin protein and human E-selectin protein, at concentrations of 10 μg/ml and 0.5 μg/ml, respectively. Unless otherwise stated the protocol used is set for in

Methods and Materials.

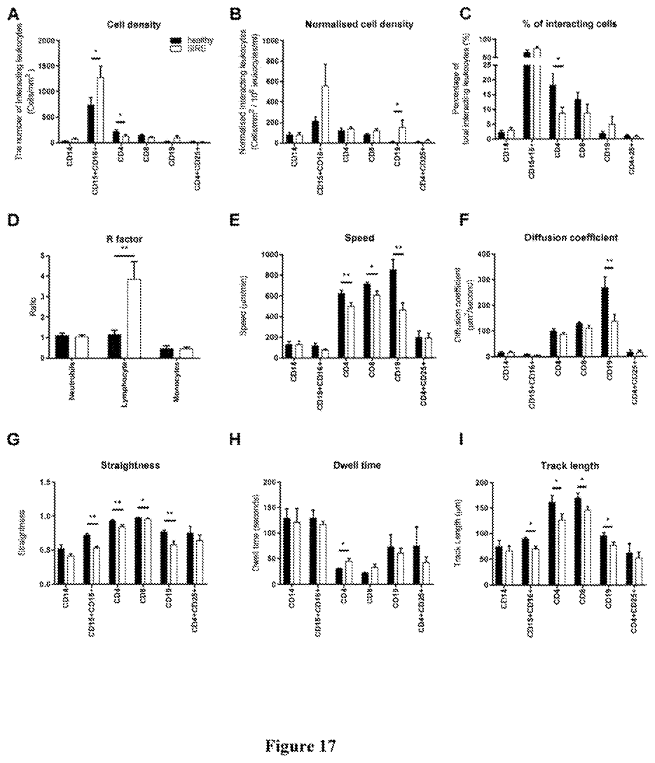

As shown in FIG. 17A, the cell density of CD15+CD16+ neutrophils is significantly higher in SIRS patients than healthy controls, suggesting an enhance PSGL-1 adhesive function on SIRS neutrophils. This notion is supported by the observation that the cell straightness and track length of CD15+CD16+ cells is lower in SIRS patients, compared with healthy controls (FIGS. 17G and 17I).

A higher lymphocyte R factor was observed in SIRS patients, compared to healthy controls, suggesting an enhanced PSGL-1 activity in SIRS lymphocytes (FIG. 17D). Consistently, the speed, straightness and track length of CD4, CD8 and CD19 lymphocytes were all significantly reduced in SIRS patients, related to healthy controls (FIGS. 17E, 17G and 17I), suggesting an stronger cell-selectin interaction and an inhibited cell mobility in SIRS lymphocytes.

As detailed in Example 7, based on their clinical records, the 14 SIRS patients were divided into three groups: 1) Non-infectious, 2) Infectious and 3) Unknown. Thus, the ability of LAFA using P-selectin plus E-selectin substrates to distinguish non-infectious SIRS from infectious SIRS was tested.

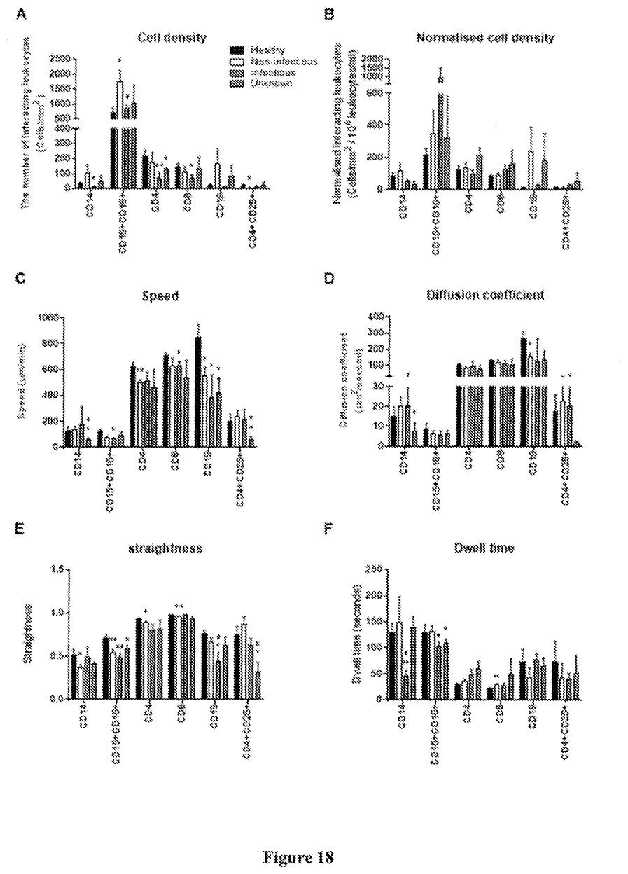

As shown in FIG. 18A, compared to healthy subjects, the cell density of CD15+CD16+ neutrophils was increased in non-infectious SIRS patients, but not in infectious SIRS group. Consistently, related to non-infectious SIRS group, the neutrophil density is significantly lower in infectious SIRS group (FIG. 18A). Similarly, the dwell time of infectious SIRS neutrophils is also lower, compared to non-infectious cells (FIG. 18F). In addition, a significantly lower dwell time was observed in infectious SIRS CD14 cells, compared with non-infectious SIRS CD14 cells, indicating a divergent cell adhesive function on CD 14 cells between non-infectious and infectious SIRS patients (FIG. 18F).

Together, these results show that a range of new markers were generated by LAFA using P-selectin plus E-selectin substrates, which may be used to determine an elevated PSGL-1 activity in SIRS patients. Additionally, the results disclosed herein indicate that a number of new LAFA markers may be used to distinguish non-infectious SIRS from infections SIRS.

### Example 13. Use Single Cell Profiles to Distinguish Different Leukocyte PSGL-1 Adhesive Function in Individual SIRS Patients by LAFA on P-Selectin Plus E-Selectin Substrates

The present Example is directed to using single cell profiles generated by leukocyte adhesive function assay (LAFA) to determine leukocyte PSGL-1 adhesive function in individual SIRS patients. Blood samples were collected from healthy volunteers and SIRS patients, and then analysed by LAFA on P-selectin plus E-selectin substrates. Unless otherwise stated the protocol used is set for in Methods and Materials.

For each blood samples collected from SIRS patients, standard microbiological testing was performed to determine potential positivity for infections. In combination of clinical records, potential causes of inflammation in individual SIRS patients was then determined by two experienced ICU specialists independently. As a result, the potential causes of systemic immune response in each SIRS patient are listed in Table 3. Accordingly, the 14 SIRS patients were divided into three groups: 1) Non-infectious, 2) Infectious and 3) Unknown, as detailed in Example 7.

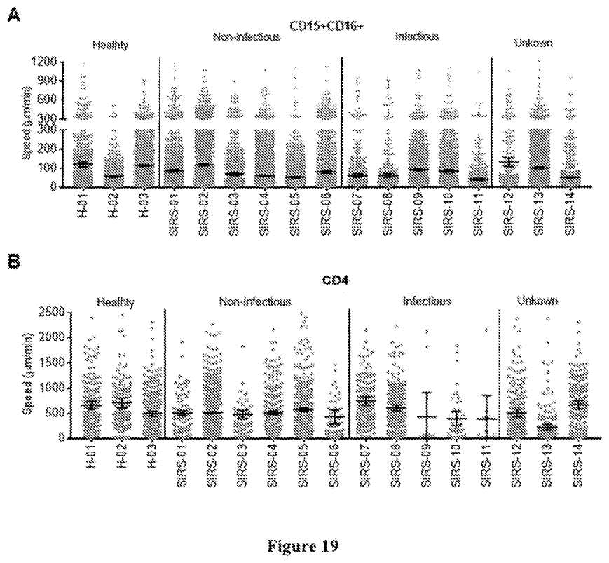

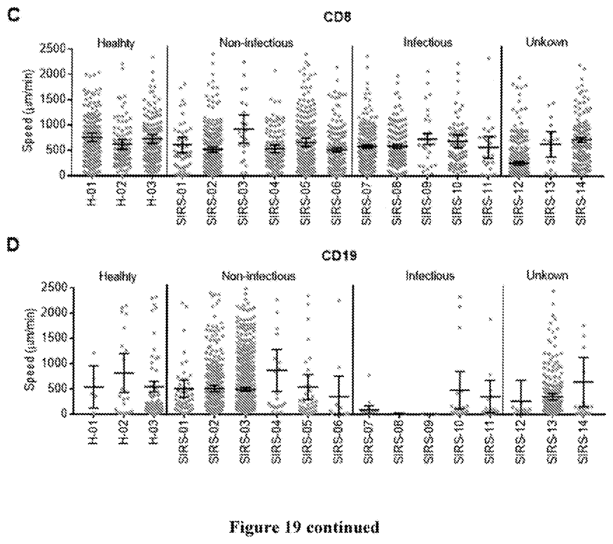

Each of the blood samples was analysed by LAFA on P-selectin and E-selectin substrates, and several cell kinetic parameters of each single interacting cell were then determined. As shown in FIG. 19D, a high density of CD19 cells was detected in patient SIRS-03 (asthma). On the other hand, only a hand full of CD19 cells was observed on VCAM-1 substrate in the same patient (FIG. 8D). These results suggest that PSGL-1 is highly activated on CD19 cells in the patient, while no obviously enhanced activity of α4β1 integrin on these cells, demonstrating divergent activation of specific adhesion molecules in this patient.

It is also noted that the number of CD4 and D8 interacting cells of patient SIRS-03 is low (FIGS. 19B and 19C). On the other hand, a large number of CD4 and CD8 cells were seen in patient SIRS-07 in this patient (enteric bacteria and Pseudomonas), whereas the CD19 cell density was low. These findings also suggest a different active status of specific cell sub-populations in individual SIRS patients.

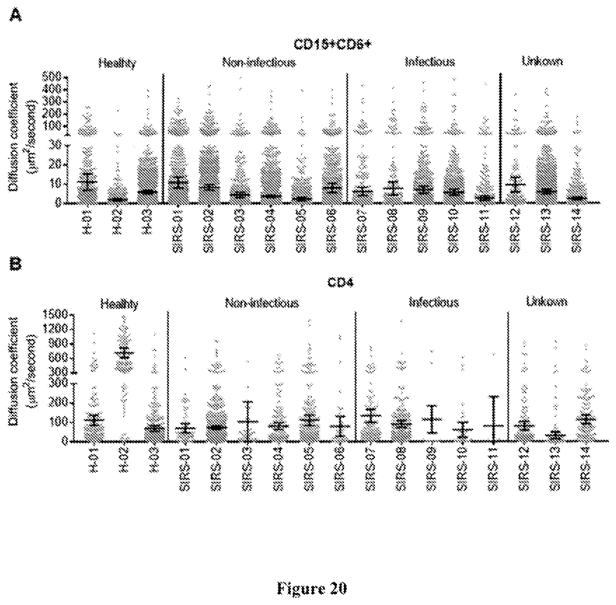

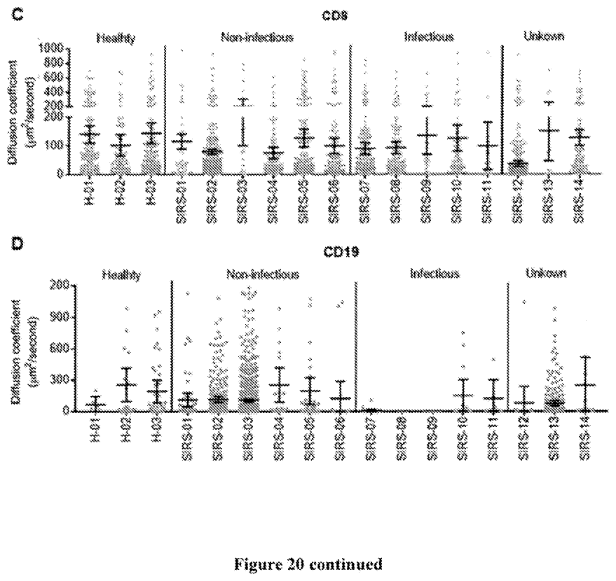

Interestingly, the CD4 cell diffusion coefficient of one of the healthy subjects (H-02) is unusually high, related to other 2 healthy subjects (FIG. 20B). In addition, the CD15+CD16+ cell diffusion coefficient in the same subject is the lowest within the healthy subjects (FIG. 20A). These findings suggest the adhesive function of different cell types may vary significantly between healthy individuals, which may be detected by LAFA.

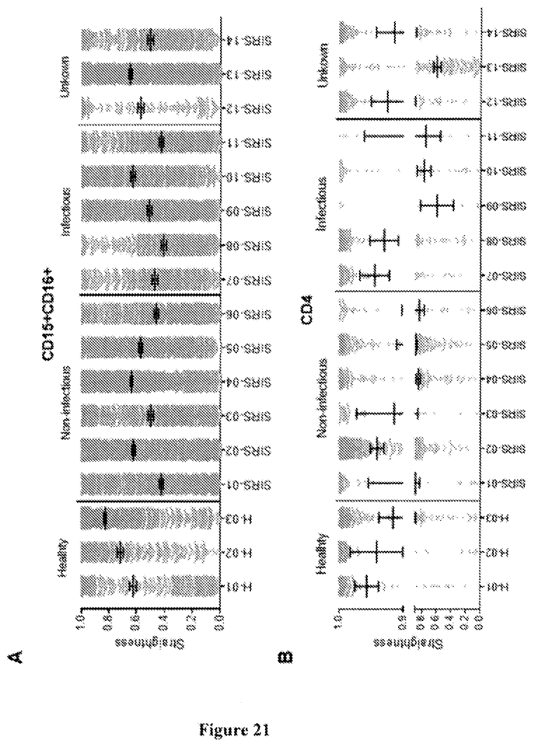

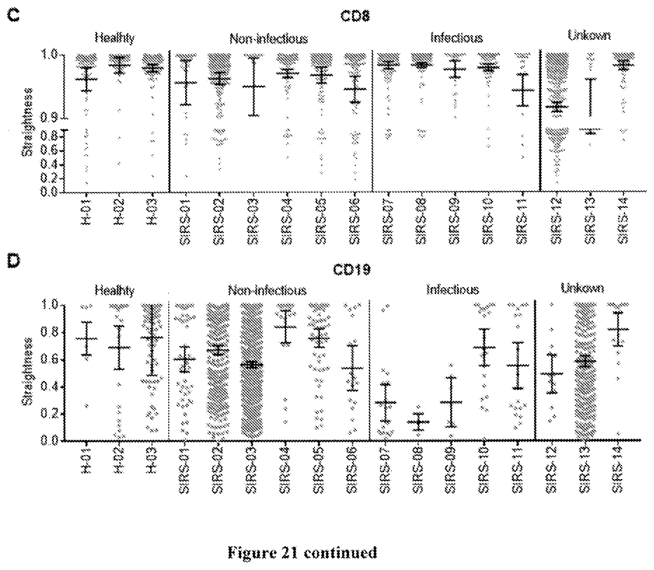

As also shown in FIG. 21A, the major portion of CD15+CD16+ neutrophil straightness in H-02 and H-03 subjects is between 0.5 and 1, whereas neutrophil straightness distributes almost evenly between 0-1 in the non-infectious SIRS patients. Moreover, the majority of the neutrophil straightness in patients SIRS-07, SIRS-08 and SIRS-11 (the infectious SIRS patients) falls between 0-0.5 (FIG. 21A), suggesting a high cell adhesive function. Similarly, a great portion of CD8 cell straightness below 0.9 was detected in patient SIRS-12, which was not seen in the other healthy and SIRS subject (FIG. 21C). These findings show different distribution patterns of cell straightness, which may be used to determine specific cell activity in individual subjects.

As also shown in FIG. 21A, the major portion of CD15+CD16+ neutrophil straightness in H-02 and H-03 subjects is between 0.5 and 1, whereas neutrophil straightness distributes almost evenly between 0-1 in the non-infectious SIRS patients. Moreover, majority of the neutrophil straightness in patients SIRS-07, SIRS-08 and SIRS-11 (the infectious SIRS patients) falls between 0-0.5 (FIG. 21A), suggesting a high cell adhesive function. Similarly, a great portion of CD8 cell straightness below 0.9 was detected in patient SIRS-12, which was not seen in the other healthy and SIRS subject (FIG. 21C). These findings show different distribution patterns of cell straightness, which may be used to determine specific cell activity in individual subjects.

### Example 14. Use Machine Learning Algorithm to Determine the Activation of Leukocyte Adhesive Functions

The present Example is directed to using a machine learning (ML) algorithm to determine the activation of leukocyte adhesive functions. Blood samples collected from healthy volunteers were treated with Mn²⁺ (activated) or without Mn²⁺ (control) for 5 minutes at room temperature, before being used for leukocyte adhesive function assay (LAFA) using VCAM-1 as substrate. The data from control and activated samples were used to train the machine learning algorithm, and the ability of the trained algorithm to distinguish blinded control and activated data was then determined.

ML is a computer science field that evolved from artificial intelligence and pattern recognition. ML algorithms enable computers to learn and make predictions from data without human input. One of ML's main applications is computer vision, a field in which computers are trained on digital images or videos to automate tasks of the human visual system.

Based on the existing LAFA images and data analysis results from healthy volunteers and patients with various diseases, a database was established. This database may keep expanding by integrating new LAFA data into the already existing database for continuous optimization of the ML algorithms.

In the first instance, a machine learning algorithm based on the convolutional neural network (CNN) method for image classification was developed. This algorithm was trained on LAFA images of Mn-activated and control blood samples of healthy donors. CNNs consist of input and output layers with a custom number of hidden layers in between. Each layer is mathematically correlated to the other layers with a weights matrix defining the mathematical relationship between the layers. For training this CNN, the raw images were processed into a standard deviation intensity projection color-coded for the different subpopulations. This CNN algorithm at present distinguishes between blinded control and Mn-activated samples with an accuracy of ˜80%. With increasing data base, the accuracy may increase to >99%.

In the second instance, a machine learning algorithm based on the Random Forest ensemble learning method was developed. This algorithm was trained on single cell tracking results (e.g. cell density, speed, diffusion coefficient, straightness, dwell time, track length etc.) of Mn-activated and control blood samples of healthy donors. Random Forest constructs a multitude of decision trees, each being trained on a different subset of the training data set. By averaging multiple independent decision trees, Random Forest lowers the risk of overfitting and thus increases the performance of the final model. This Random Forest algorithm at present distinguishes between control and Mn-activated samples with an accuracy of >80%. With an increasing data base, the accuracy may increase to >99.9%.

Comparing the two algorithms, currently, the accuracy is similar. The Random Forest algorithm may be more accurate than the CNN as it may learn on a wider range of data (tracking parameters, may also include images) but more time consuming to train and classify as tracking analysis may need to be performed beforehand. CNN may be quicker both for training and classification as little data pre-processing is required. Accuracy may be lower than that of Random Forest algorithm, however, a larger database is required to determine the limitations of each of the algorithms.

### Example 15. Assess the Effects of Natalizumab on VCAM-1 Dependent Leukocyte Recruitment Using LAFA on VCAM-1 Substrate in SIRS Patients

The present example is directed to using leukocyte adhesive function assay (LAFA) on VCAM-1 substrate to assess Natalizumab (Biogen, MA) effects on leukocyte recruitment in SIRS patients. Blood samples were collected from healthy volunteers and SIRS patients and treat with Natalizumab (30 μg/ml) for 5 minutes at room temperature, before being analysed by LAFA on VCAM-1 substrate. Unless otherwise stated the protocol used is set out in the Methods and Materials.

Natalizumab, marketed by Biogen as Tysabri, is a neutralising monoclonal anti-human α4β1 integrin antibody and is one of the most effective multiple sclerosis (MS) therapies. Natalizumab was originally developed to block α4β1 integrin functions and suppress leukocyte adhesive function in MS patients, leading to a reduced leukocyte infiltration across the blood brain barrier. As shown in FIG. 6 and Example 6, an enhanced activation of α4β1 integrin was detected in SIRS patients. Thus, the effect of Natalizumab on α4β1 function and VCAM-1 dependent leukocyte recruitment was then investigated in SIRS patients.

A shown in FIG. 22A, compared with untreated controls, Natalizumab treatments significantly reduced the number of interacting CD15+CD16+ neutrophils in healthy subject and in non-infectious SIRS patient, suggesting the ability of Natalizumab to suppress α4β1 integrin function in these cells. However, Natalizumab treatments failed to have such inhibitory effect in infectious SIRS patients (FIG. 22A). In addition, Natalizumab significantly inhibited CD4 and CD8 cell recruitment in healthy and the three SIRS groups, in line with the known action of Natalizumab (FIGS. 22B and 22C).

Together, these results suggest Natalizumab may inhibit leukocyte α4β1 integrin function in multiple cell sub-populations, resulting in suppression of leukocyte recruitment. These finding also demonstrate the ability of LAFA to test drug effects on leukocyte adhesive function in vitro. Based on the results from LAFA, potential responses of individual subjects to specific drugs/therapies may be projected, facilitating the development of optimised therapies for individual patients.

### Example 16: Assess Serum C-Reactive Protein in SIRS Patients

The present Example is directed to measuring serum C-reactive protein (CRP) levels in SIRS patients, and the ability of serum CRP levels to distinguish non-infectious SIRS from infectious SIRS may be determined. Blood samples were collected from healthy volunteers and SIRS patients. The serum from each blood sample was centrifuge at 2,000 g for 10 minutes at 4° C. so that the blood serum (supernatant) was then collected. The concentrations of CRP were determined by commercial ELISA kits, according to the manufacturer's instructions (ThermoFisher Scientific).

For each blood sample collected from SIRS patients, standard microbiological testing was performed to determine potential positivity for infections. In combination of clinical records, potential causes of inflammation in individual SIRS patients was then determined by two experienced ICU specialists independently. As a result, the potential causes of systemic immune response in each SIRS patient are listed in Table 3. Accordingly, the 14 SIRS patients were divided into three groups: 1) Non-infectious, 2) Infectious and 3) Unknown, as detailed in Example 7.

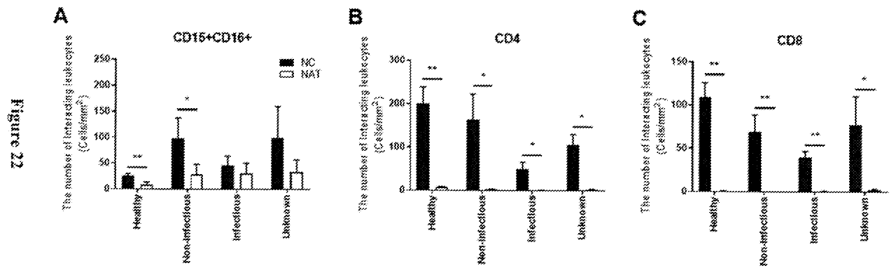

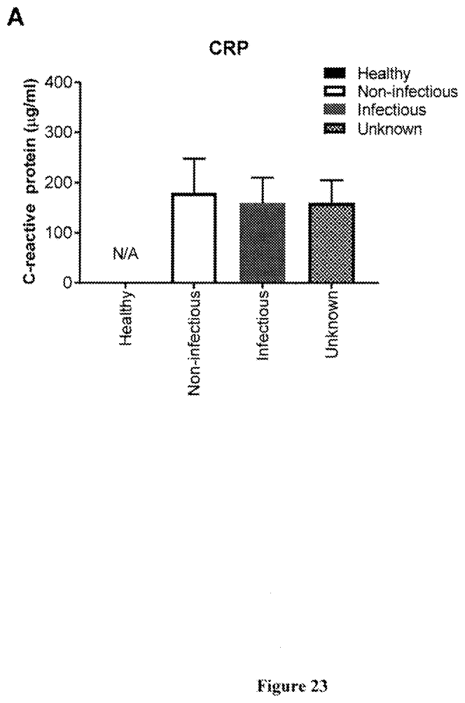

As shown in FIG. 23A, the CRP levels in the healthy subjects are below the detection limit of the ELISA kits. No differences in CRP levels between the three groups of SIRS patient was detected (FIG. 23A), suggesting CRP is not a suitable marker to distinguish non-infectious SIRS from infectious SIRS patients.

### Example 17. Assessing Leukocyte Adhesion Function in SIRS Patients by IAEA Using MAdCAM-1 as Substrate

The present Example is directed to testing the ability of leukocyte adhesive function assay (LAFA) to detect an elevated immune response in patients with systematic inflammatory response syndrome (SIRS) using MAdCAM-1 as a substrate. Blood samples were collected from healthy and SIRS subjects, and then analysed by LAFA on MAdCAM-1 substrate. The microfluidic channels were pre-coated with human MAdCAM-1 protein at concentrations of 14 μg/ml. Unless otherwise stated the protocol used is set for in Methods and Materials.

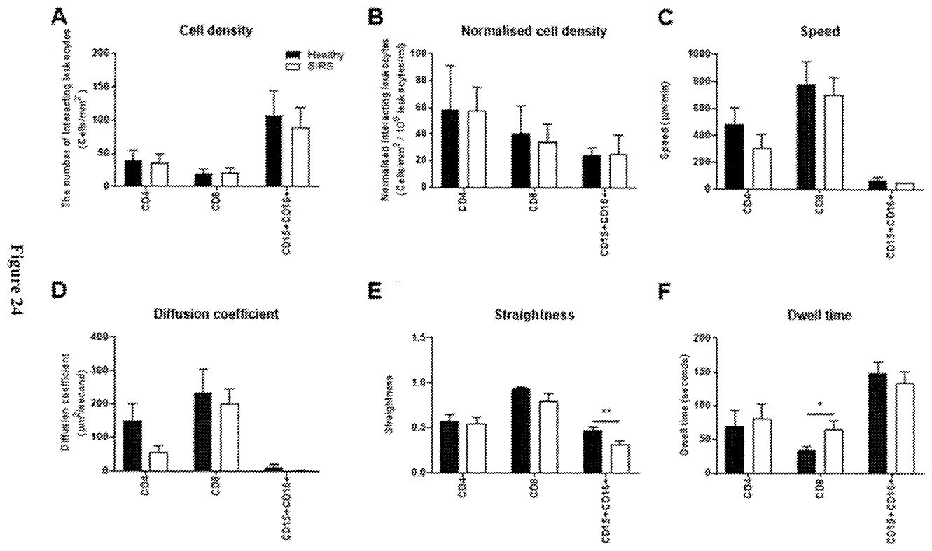

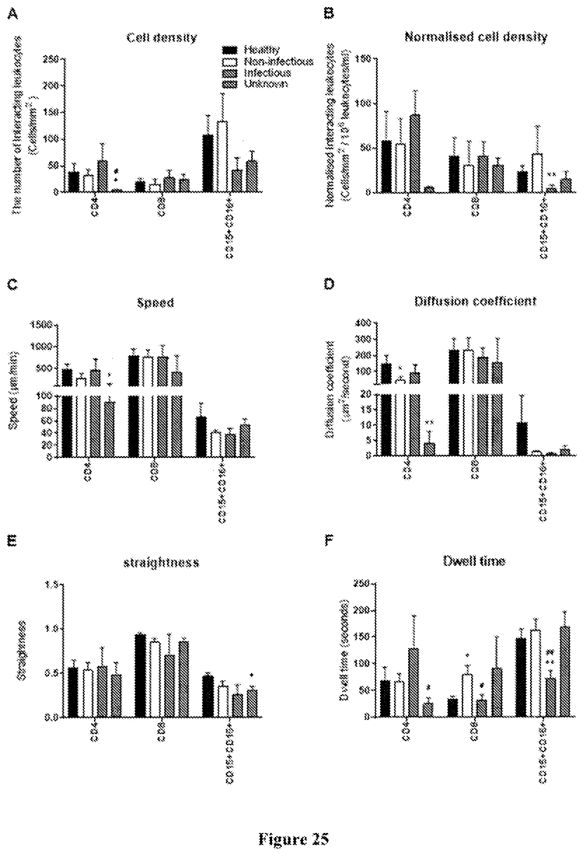

As shown in FIGS. 24A and 24B, no difference in cell density in CD4, CD8 and CD15+CD16+ interacting cells were detected with or without being normalised by appropriate cell counts. Compared to healthy subjects, on the other hand, a significantly lower straightness in CD15+CD16+ neutrophils was observed in SIRS patients (FIG. 24E), suggesting a enhance α4β7 integrin activity related to healthy subjects. In addition, the dwell time of CD8 cells was significantly higher in SIRS patients than healthy subject (FIG. 25F).

As detailed in Example 7, based on their clinical records, the 14 SIRS patients were divided into three groups: 1) Non-infectious, 2) Infectious and 3) Unknown. Thus, the ability of LAFA (MAdCAM-1) to distinguish non-infectious SIRS from infectious SIRS was tested.

A significantly lower normalised CD15+CD16+ neutrophil density was detected in infectious SIRS patients, related to healthy subjects (FIG. 25B), suggesting a reduced α4β7 integrin adhesive function in infectious SIRS patients. This notion is supported by the findings showing a significantly lower dwell time in infectious SIRS patients related to healthy controls and non-infectious SIRS patients (FIG. 25F). In addition, the CD8 cell dwell time was significantly lower in infectious SIRS patients compared with infectious SIRS patients (FIG. 25F). These suggest a potential to use these new markers to separate non-infectious SIRS from infectious SIRS.

Mn²⁺, a pan integrin activator, activates α4β7 integrin on leukocyte membrane and, therefore leads to an increased α4β7 integrin binding activity to its endothelial ligand, MAdCAM-1. To determine different effects of Mn²⁺ on α4β7 integrin activation in healthy and SIRS subjects, the blood samples were treated with 5 mM MnCl₂ for 5 minutes at room temperature before being used for LAFA on MAdCAM-1 substrate.

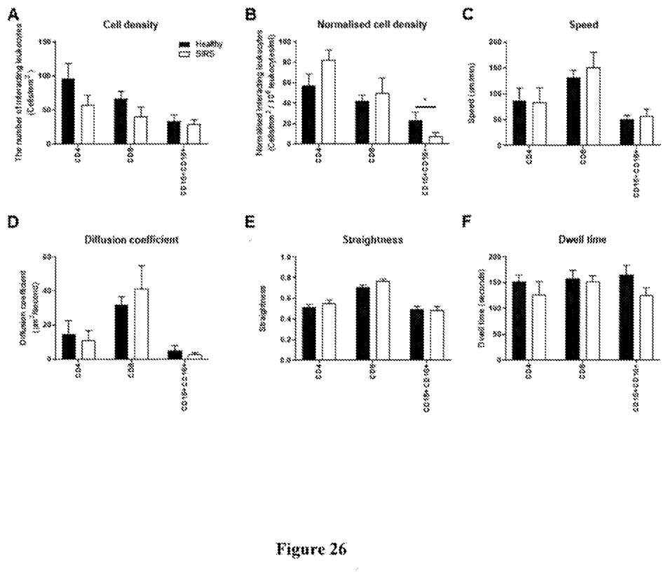

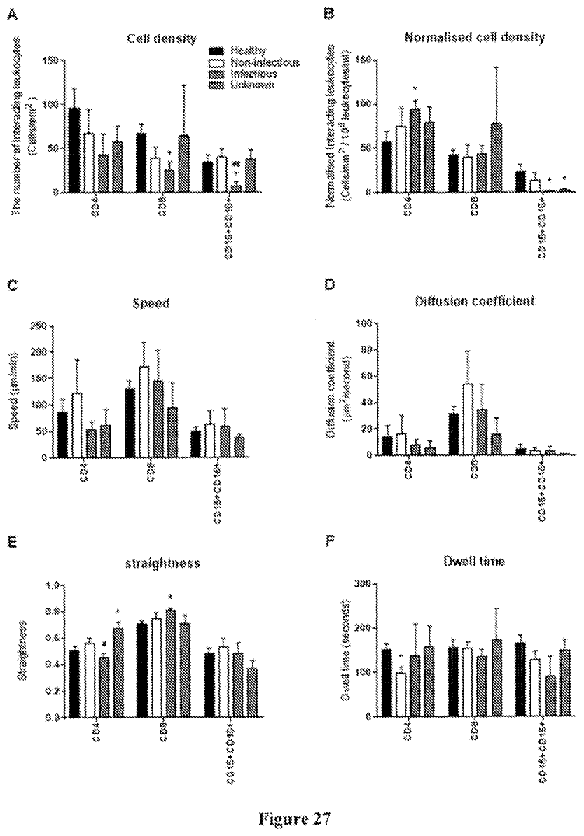

In the presence of Mn²⁺, a lower normalised cell density of CD15+CD16+ neutrophils was detected in SIRS patients than healthy subjects (FIG. 26B). After being divided into three groups, it was found that the neutrophil cell density was significantly lower in infectious SIRS patient than non-infectious patients (FIG. 27A). In addition, the straightness of CD4 cells in infectious SIRS was determined to be significantly lower in infectious SIRS than non-infectious SIRS (FIG. 27E).

Together, these findings show, for example, the ability of LAFA on MAdCAM-1 substrate to generate a range of new markers to determine different α4β7 integrin response to Mn²⁺ in healthy and three SIRS groups. LAFA has identified a number of useful cell kinetic markers that may be used to distinguish non-infectious SIRS patients from infectious SIRS patients.

### Example 18, Pre-Symptomatic Detection of Influenza Infection

A healthy adult subject (CIN-001) was recruited to the study for the purpose of providing a healthy control sample. At the time of presenting for collection of a first blood sample on a Thursday (Day 0), the subject reported as being healthy and had no symptoms of viral infection. In the evening of the same day, after the initial blood sample was taken, the subject developed a sore throat (pharyngitis). On the next day (Day 1), the subject started to have typical symptoms of influenza infection, including coughing and difficulty breathing. A second blood sample was collected on Day 5 (Tuesday) and used for the LAFA assay when the influenza symptoms were more severe. On the same day (Day 5), the subject was diagnosed with suspected influenza by a general practitioner (GP). The influenza symptoms lasted about two weeks. After this, the subject was fully recovered and remained healthy. A third blood sample was collected and analysed by LAFA on a Tuesday eleven weeks after the second blood sample was taken. Thus, the third blood sample was used as a healthy base line sample.

As shown in Table 4, an incremental increase of total white blood cells counts (WBC) were detected and WBC reached the highest in the third blood sample when the flu symptoms were severe. A similar increase was seen in a number of leukocyte subsets except for lymphocytes. The highest lymphocyte count was detected at the incubation time (the second blood sample) and the lymphocyte counts of the first and the third blood samples are comparable.

TABLE 4
The total white blood cell counts of three blood samples, base
line, incubation time and viral induced flu. Blood samples
were collected and full blood cell counts were performed.

| | Cell counts (million cells/ml) | | | | |
|---|---|---|---|---|---|
| | Total | Neu | Lym | Mono | Eos |
| Healthy basal | 6.26 | 3.91 | 1.53 | 0.46 | 0.33 |
| Incubation | 7.41 | 4.03 | 2.19 | 0.65 | 0.52 |
| Viral flu | 8.05 | 5.09 | 1.31 | 0.75 | 0.89 |

Neu: neutrophils, Lym: lymphocytes, Mono: monocytes, Eos: eosinophils.

When the blood samples were analysed by LAFA on P+E selectin substrates, the speed and diffusion coefficient (FIGS. 28A and 28B) of CD4 and CD8 interacting cells were both reduced in the second and third blood samples compared to the basal healthy blood cells, indicating a virus-induced selectin P ligand (PSGL-1) activation in these adaptive immune cells. A similar activation of PSGL-1 on CD14 and CD15+CD16+ cells were also detected in the second and the third blood samples, as a decrease of cell speed, diffusion coefficient, straightness track length and displacement was detected (FIG. 28). These findings show an activation of innate and adaptive immune cells during the incubation period and the symptomatic influenza period.

Compared to basal cells, the cell straightness of CD15+CD16+ neutrophils was significantly lower during incubation time (the second blood sample), which was not detected when the influenza symptoms were severe (the third blood sample) (FIG. 28C). These data show CD15+CD16+ straightness may be used as a unique LAFA marker to identify potential viral infection in people who have been infected, but have not yet had obvious influenza symptoms.

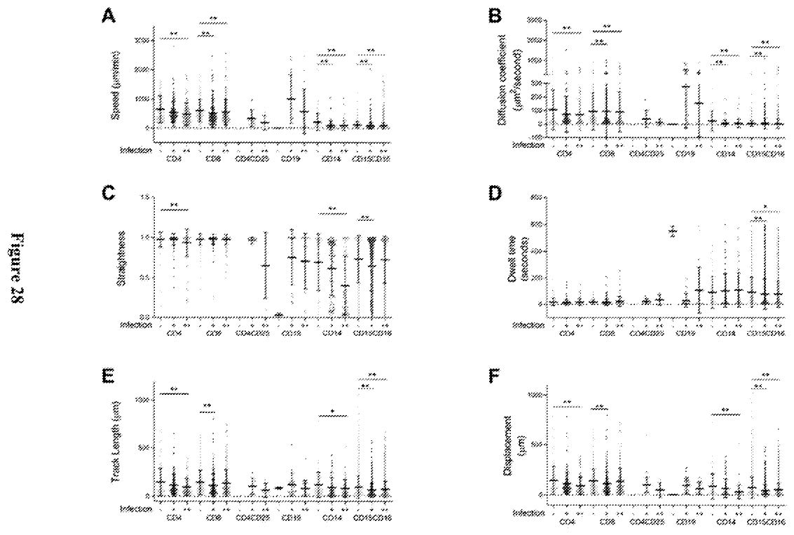

When the blood samples were analysed by LAFA using VCAM-1 as substrate, a decreased speed, diffusion coefficient, straightness and displacement (FIG. 29) of CD4 and CD8 interacting cells were detected in the second blood sample (incubation period) compared to the basal cells, suggesting an activation of α4β1 integrin due to the viral infection. In the third blood sample where the influenza symptoms were severe, the number of interacting CD4 and CD8 cells decreased to almost zero (FIG. 29) even though the lymphocyte counts were comparable between the first and the third blood samples. These results show that the CD4 and CD8 cells in the third blood sample almost completely lost α4β1 integrin function, possibly due to an anti-inflammatory response induced by the immune system aiming to attenuate virus-induced immune activation. The fact that the loss of α4β1 integrin did not occur during the incubation time shows the status of the immune system significantly differs between incubation period with no influenza symptoms and the time when influenza symptoms were observed.

Together, these data demonstrate the ability of LAFA to detect not only the effects of viral infection on the immune system, but also the different responses of the immune system to the pathogen at different stages of infection. Thus, the LAFA biomarkers may be used to detect early signs of infections during the incubation period, which may not usually be detected by other routine blood tests. Moreover, the same individual may respond to different pathogens differently, while other individuals may also respond to the same foreign pathogen differently. LAFA is ideal to detect such differences, allowing early detection of infection and facilitating the development of optimal treatments based on the divergent immune status in individual patients.

### Example 19. Evaluating Systemic Inflammatory Response Syndrome (SIRS) by LAFA Measurement of Leukocyte Adhesive Function on P+E Selectin as Adhesive Substrates

LAFA measurement of leukocyte adhesive function on P+E selectin adhesive substrate was used to evaluate SIRS from patient samples. The ability of LAFA to distinguish infectious and non-infectious SIRS was also assessed. In Example 12, blood samples from 14 SIRS patients were analysed by LAFA on P+E selectin substrates. In the present example, samples from an additional 14 SIRS patients were analysed and included in the data analysis. The data presented in this example includes 28 SIRS patients. Based on patients' clinical records (Table 5), each of the new 14 SIRS patients were retrospectively assessed to determine if it represents either:
1. Group “Non-infectious”: Infection UNLIKELY or
2. Group “Infectious”: PROVEN infection (e.g. Positive microbiology result) or
3. Group “Unknown”: Infection possible BUT not proven.

TABLE 5
The causes of systemic inflammatory response in the additional
14 SIRS patients. Blood samples were collected from each of
the SIRS patients and standard microbiological testing was
performed to determine potential infection. In combination
with clinical records, the causes of inflammation in individual
SIRS patients were then determined as mentioned above.

| Groups | Codes | Causes of inflammation |
|---|---|---|
| Healthy | H-04 | Healthy #04 |
| | H-05 | Healthy #05 |
| | H-06 | Healthy #06 |
| Non-infectious SIRS | SIRS-15 | Trauma |
| | SIRS-16 | Cancer |
| | SIRS-17 | Surgery |
| | SIRS-18 | Surgery |
| Infectious SIRS | SIRS-19 | Endocarditis |
| | SIRS-20 | Tissue infection |
| | SIRS-21 | Gram-negative |
| | SIRS-22 | Chest infection |
| | SIRS-23 | E. coli in peritoneum |
| Unknown | SIRS-24 | Unknown |
| | SIRS-25 | Possible infection |
| | SIRS-26 | Possible surgery |
| | SIRS-27 | Possible surgery |
| | SIRS-28 | Possible trauma |

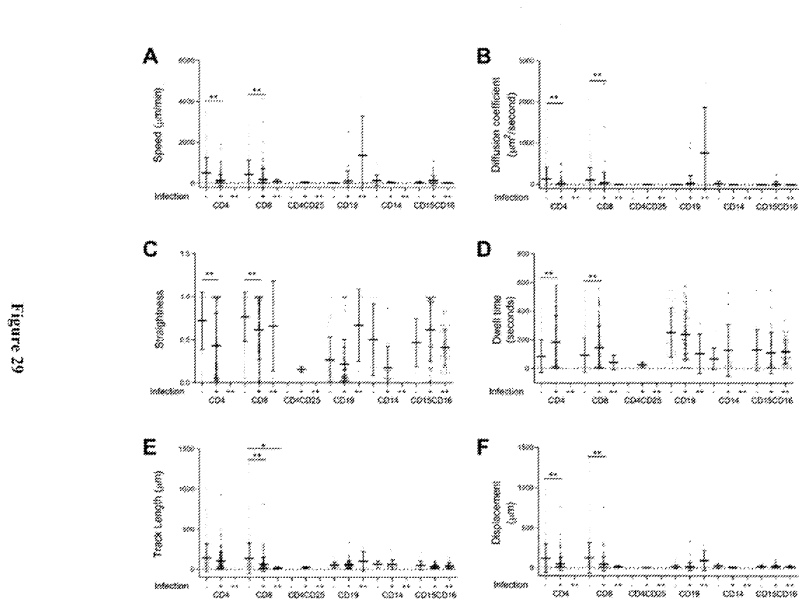

As shown in FIG. 30I, SIRS patients had a significantly higher total white blood cell counts related to healthy subjects (n=14), which is predominantly due to an enhanced neutrophil count. The lymphocyte counts are significantly lower in infectious SIRS patients, comparing to both healthy subjects and non-infectious patients. The monocyte counts in non-infectious patients was higher than healthy subjects.

The number of interacting CD4 and CD8 cells was significant lower in infectious SIRS patients (FIG. 30A), compared to both healthy subjects and non-infectious patients, possibly due to the lower lymphocyte counts (FIG. 30I). The straightness and displacement of CD4 interacting cells in infectious SIRS patients were significantly lower than healthy subjects and non-infectious patients, showing an activated PSGL-1 in infectious patients. Consistently, the dwell time of infectious CD4 cells is significantly increased related to healthy subjects and non-infectious patients. These findings show that PSGL-1 activation is a unique marker in infectious SIRS patients, which may be used to distinguish infectious SIRS patients from non-infectious. Additionally, a significantly greater CD4+CD25+ interacting cells were found in infectious patients related to other groups, while the cell migratory behaviours are similar in all groups.

The straightness (FIG. 30E), track length (FIG. 30G) and displacement (FIG. 30H) of interacting CD15+CD16+ cells (neutrophils) were all reduced in all SIRS patient groups (including non-infectious, infectious and unknown), compared to healthy subjects. These results suggest that an increased PSGL-1 activity in the SIRS patients, which may be used as a marker to detect systemic inflammatory responses.

For each of the blood samples collected from the additional 14 SIRS patients, standard microbiological testing was performed to determine potential positivity for infections. In combination with clinical records, potential causes of inflammation in individual SIRS patients was then determined by two experienced ICU specialists independently. As a result, the potential causes of systemic immune response in each SIRS patient are listed in Table 5. Accordingly, the 14 SIRS patients were divided into three groups: 1) Non-infectious, 2) Infectious and 3) Unknown, as detailed above.

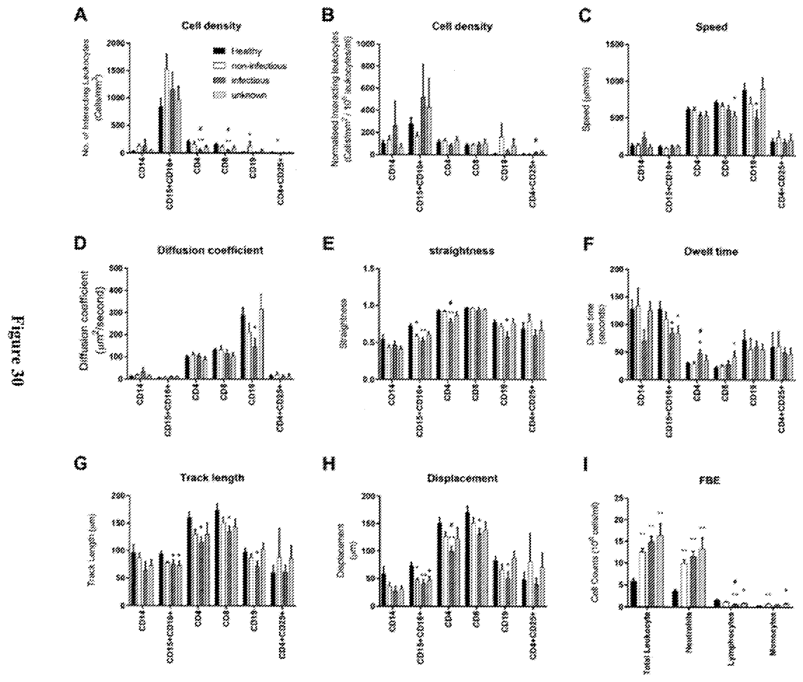

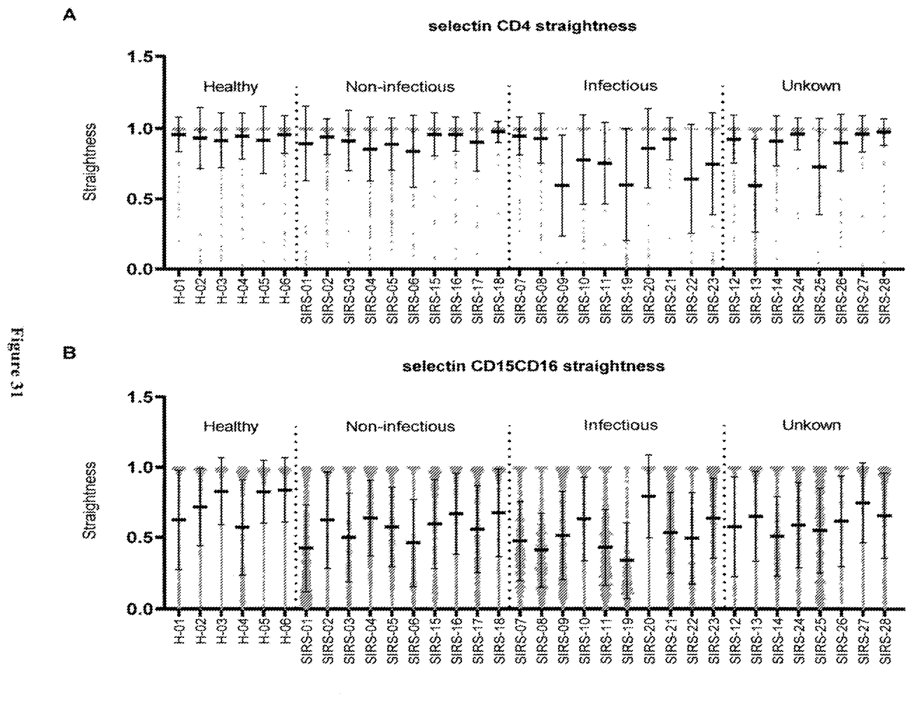

For these blood samples (including 6 new healthy subjects and 28 SIRS patients), a number of cell kinetic parameters (e.g. cell density, speed, diffusion coefficient, straightness, dwell time, track length and displacement) were determined for single interacting cells so that single cell profiles were generated for specific leukocyte subsets. As shown in FIG. 31A, the average of CD4 cell straightness is high (close to 1) in the healthy subjects and non-infectious SIRS patients, whereas the CD4 straightness values of most infectious patients are low, providing a useful LAFA marker to distinguish infectious patients from non-infectious. For SIRS patient SIRS-25, both ICU specialists scored this patient as possible infection even though no evidence of infectious pathogens was found. Given a low CD4 straightness was detected in patient SIRS-25 (FIG. 31A), patient SIRS-25 is likely to be an infectious patient. For the same reason, patient SIRS-13 may also be an infectious patient.

Additionally, as shown in FIG. 31B, the number of interacting CD15+CD16+ cells in healthy blood samples is lower than SIRS patient samples. The cell the average values of CD15+CD16+ cell straightness of healthy subjects are higher than the values of SIRS patients. These results suggest these LAFA marker may be used to distinguish healthy subjects from SIRS patients.

Together, these results demonstrate the ability of LAFA to generate useful LAFA markers on selectin substrate to not only detect systemic inflammatory responses, but also distinguish infectious SIRS patients from non-infectious patients. A combination of these LAFA markers may increase the accuracy and sensitivity of the LAFA assays as a diagnostic test for SIRS or sepsis. The accurate assessment of immune system activation by LAFA may provide useful information on how immune system respond to inflammatory stimuli on a personal basis, facilitating the development of optimised treatments.

### Example 20. Evaluating Systemic Inflammatory Response Syndrome (SIRS) by LAFA Measurement of Leukocyte Adhesive Function on VCAM-1 as Adhesive Substrate

In Example 6, blood samples from 14 SIRS patients were analysed by LAFA on VCAM-1 substrate (the ligand for leukocyte α4β1 integrin). In the present Example, 14 additional new SIRS patients were recruited to the analysis. The ability of LAFA to distinguish infectious and non-infectious SIRS was then assessed. The data presented in this example includes all 28 SIRS patients (14 initial and 14 additional patients). Based on patients' clinical records (Table 5), the 14 additional SIRS patients were retrospectively assessed to determine if it the SIRS was either 1) non-infectious, 2) infectious or 3) unknown.

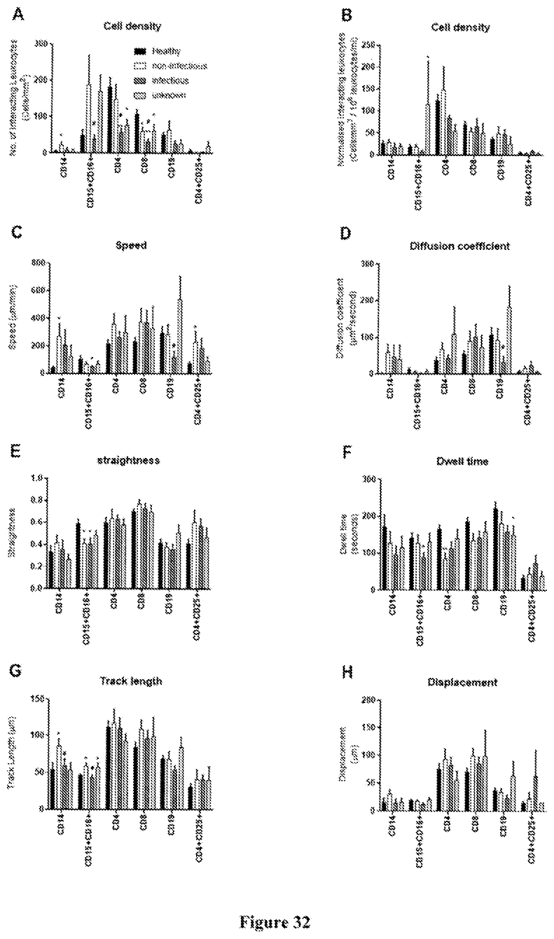

As shown in FIG. 32A, the number of interacting CD4 and CD8 cells on VCMA-1 substrate was significantly lower in infectious SIRS patients, comparing to both healthy subjects and non-infectious SIRS patients. However, no such difference was detected after the cell density was normalised by correspondent leukocyte counts (FIG. 32B).

The speed (FIG. 32C) and diffusion coefficient (FIG. 32D) of interacting CD19 cells is significantly lower in infectious SIRS patients related to both healthy subjects and non-infectious SIRS patients, suggesting a specific α4β1 integrin activation on CD19 cells from infectious patients. The speed (FIG. 32C) and straightness (FIG. 32D) of CD15+CD16+ cells were reduced in both non-infectious and infectious SIRS patients, comparing to healthy subjects. The track length of CD15+CD16+ cells were comparable between healthy subjects and infectious patients, whereas the track length of CD15+CD16+ cells was significantly longer than healthy subjects and infectious patients (FIG. 32G) Similarly, a significantly longer track length of CD14 cells was detected in non-infectious SIRS patients, comparing to healthy subjects and infectious patients (FIG. 32G). These findings show that these LAFA markers may be used in combination to either identify patient with SIRS or distinguish infectious SIRS from non-infectious. Other combinations of LAFA markers may also be useful to either identify patient with SIRS or distinguish infectious SIRS from non-infectious.

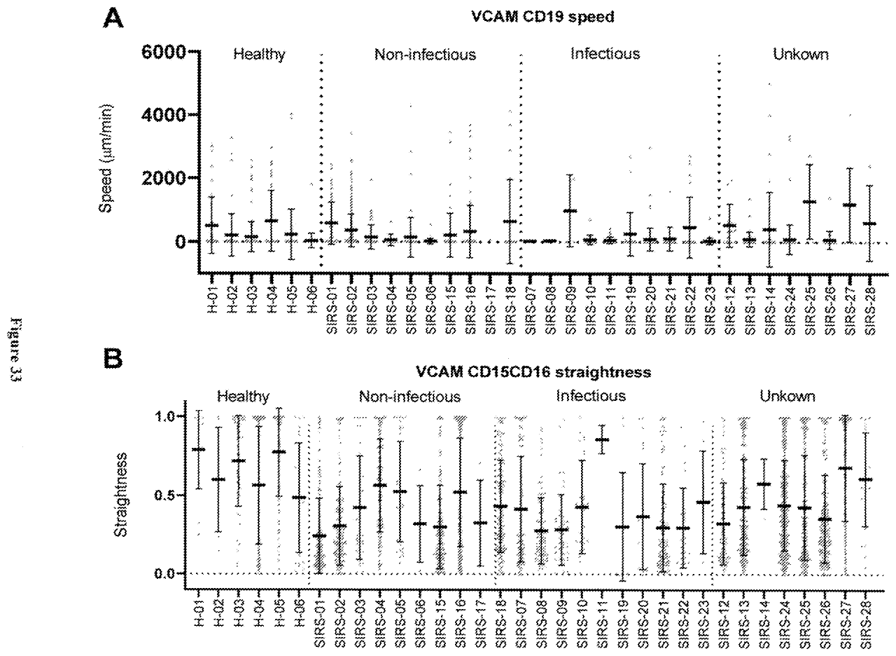

Single cell profiles for the leukocyte subsets in the blood samples were also produced. As shown in FIG. 33A, the majority of CD19 cell speed in infectious SIRS patients is low, compared to healthy and non-infectious groups. Additionally, the CD15+CD16+ cell straightness of healthy subject is mostly higher (closer to 1) compared to the SIRS patients (FIG. 33B).

Together, these results demonstrate the ability of LAFA to generate useful LAFA markers on VCAM-1 substrate to not only detect systemic inflammatory responses, but also distinguish infectious SIRS patients from non-infectious patients. A combination of these LAFA markers may increase the accuracy and sensitivity of the LAFA assays as a diagnostic test for SIRS or sepsis. The accurate assessment of immune system activation by LAFA may provide useful information on how immune system respond to inflammatory stimuli on personal basis, facilitating the development of optimised treatments.

### Example 21. the Effects of SIRS on Leukocyte Adhesive Function as Measured by LAFA Using VCAM-1 Plus IL-8 and VAM-1 Plus SDF-1α as Adhesive Substrates

The present example is directed to detecting SIRS effects on leukocyte adhesive function as measured by LAFA using VCAM-1 plus IL-8 or VCAM-1 plus SDF-1α substrates. In Examples 9 and 10, 14 SIRS patients were analysed by LAFA using VCAM-1 plus IL-8 and VCAM-1 plus SDF-1α, respectively. In the present example, 14 additional SIRS patients were recruited to the analysis. The ability of LAFA to distinguish infectious and non-infectious SIRS was then assessed. The data presented in this example includes the 28 SIRS patients (14 original and 14 additional patients).

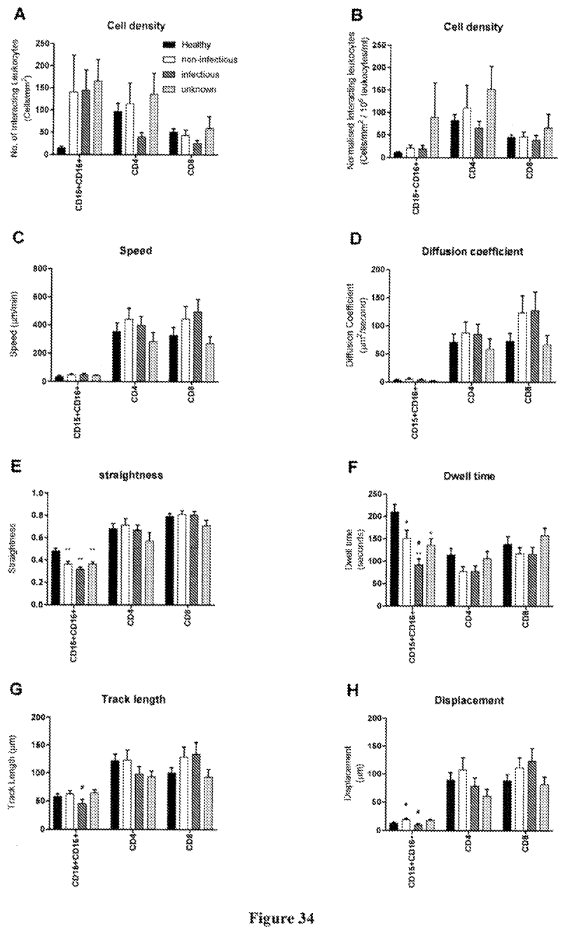

When the blood samples were analysed by LAFA on VCAM-1 plus IL-8 substrates, the straightness of CD15+CD16+ interacting cells in the SIRS patients was significantly lower than the healthy subjects (FIG. 34E), indicating an activation of CXCR1 in SIRS CD15+CD16+ cells. Consistently, the dwell time of CD15+CD16+ cells in the SIRS patients was significantly lower than healthy subjects, while the CD15+CD16+ cell dwell in infectious SIRS patients is also significantly lower than non-infections patients (FIG. 34F).

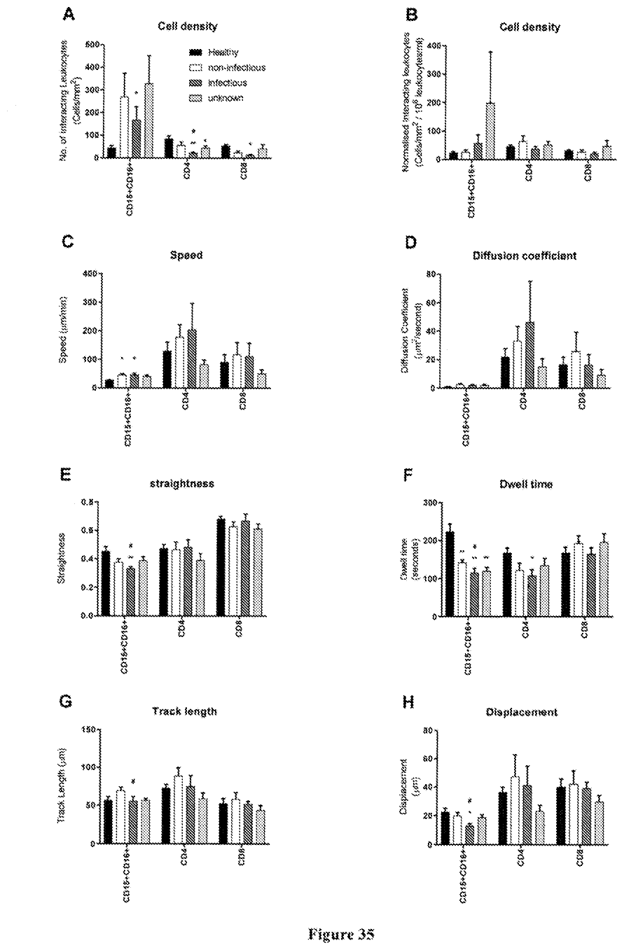

When the blood samples were analysed by LAFA on VCAM-1 plus SDF-1α substrates, the number of interacting CD4 cells was significantly lower in infectious patients compared to healthy subjects and non-infectious patients (FIG. 35A). The dwell time of infectious CD4 cells was significantly lower than healthy subjects, (Figure F) showing a greater activity of CXCR4 in infectious CD4 cells. Additionally, the speed of CD15+CD16+ cells from infectious and non-infectious SIRS patients was significantly higher than healthy cells, whereas the straightness of CD15+CD16+ cells from infectious SIRS patients was significantly lower than healthy and non-infectious cells (Figure E). These results show a divergent activity of CXCR4 on infectious CD15+CD16+ cells, leading to reduced cell mobility, related to healthy and non-infectious cells. Similarly, the dwell time, and displacement of infectious CD15+CD16+ cells were significantly lower than healthy and non-infectious cells (FIGS. 35F and 35H).

Together, these findings show different activities of CXCR1 and CXCR4 in healthy subjects, non-infectious and infectious SIRS patients. Thus, these LAFA markers may be used to not only identify systemic inflammation in SIRS patients, but also distinguish infectious SIRS patients from non-infectious patients. A combination of these LAFA markers may increase the accuracy and sensitivity of the LAFA assays as a diagnostic test for SIRS or sepsis. The accurate assessment of immune system activation by LAFA may provide vital information on how immune system respond to inflammatory stimuli on personal basis, facilitating the development of optimised treatments.

### Example 22. LAFA Markers

The present example provides a non-exclusive list of LAFA markers that may be generated from the analysis of LAFA assays.

One or more images were analysed by the image analysis software, as described in Example 3. The positions of a cell were then determined in one or more frames by the software (Example 3). Using this cell position data, the LAFA markers are calculated.

In this example, the LAFA marker is categorised by adhesive substrates (as described in Table 2 Example 1), activation status (e.g. with or without Mn²⁺, Examples 4 and 5) and measurement category (Table 6). All LAFA cell markers are listed in Tables 7 and 8. All LAFA markers may be determined for each leukocyte subsets (as described in Table 1 in Example 1).

The markers listed in Table 8 are derived from cell instantaneous speeds. In certain embodiments, instantaneous speed may be defined as where each movement distance recorded between one frame to the next of a particular cell. For example, a cell that is recorded for 100 frames will result in 99 instantaneous speeds. Apart from cell number and normalised cell number, a mean of a marker from all cells in a specific leukocyte population may be calculated for all the parameters listed in Table 7 and Table 8.

TABLE 6
List of measurement categories.

| Measurement categories | Explanation |
|---|---|
| General | Refer to all the cells detected within the |
| | channel regardless of the cell duration |
| Short durations | Refer to cells that stayed for short period |
| | of time, for example 3-30 frames |
| Medium durations | Refer to cells that stayed for medium period |
| | of time, for example 31-150 frames |
| Long durations | Refer to cells that stayed for long period of |
| | time, for example 150-300 frames plus |

TABLE 7
List of LAFA markers of measurement in regarding the overall behaviour of
each recorded cell.

| Markers | Explanation |
|---|---|
| Cell number | The number of cells detected as a valid interaction (Example 3) |
| Normalised cell | Number of cells normalised to the blood count of per 1 ml of blood (e.g. |
| number | 10⁶ of lymphocytes or neutrophils or monocytes) |
| General speed | The distant over time of at least a recorded cell |
| Dwell time | The total duration of at least a recorded cell |
| Displacement | The distant between the beginning position and the end position of at |
| | least a recorded cell |
| Track length | The total length travelled of at least a recorded cell |
| Straightness | The ratio of displacement over track length of at least a recorded cell |
| Diffusion coefficient | The diffusion coefficient is calculated as mean square displacement |
| | divided by 4 times of the time the cell travelled of at least a recorded |
| | cell |
| Stickiness index | A function that ranges between 0 and 100, and 100 refers to the |
| | maximum stickiness of a cell. The value of the function depends on the |
| | interaction of three variables: displacement, duration, and maximum |
| | speed. The interactions are displacement-duration, and displacement- |
| | maximum speed. |
| | $Index = 75 + 25 ( \frac{Duration}{Displacement + Duration} ) - 75 ( \frac{MaxSpeed + Displacement}{550} )$ |
| | This means the stickiness index increases with increased Duration and |
| | decreases with increased Displacement and MaxSpeed. |

TABLE 8
LAFA markers derived from instantaneous speeds.

| Markers | Explanation |
|---|---|
| Individual mean | The mean of instantaneous speeds of a |
| speed | recorded cell. |
| Individual median | The median of instantaneous speeds of |
| speed | a recorded cell. |
| Individual maximum | The maximum instantaneous speed of a |
| speed | recorded cell |
| Individual minimum | The minimum instantaneous speed of a |
| speed. | recorded cell |
| High speed | The percentage of instantaneous speeds |
| | that are greater than the median |
| | instantaneous speed of a recorded cell. |
| High+ speed | The percentage of instantaneous speeds |
| | that are greater than the third quartile |
| | instantaneous speed of a recorded cell. |
| High log10 speed | The percentage of instantaneous speeds |
| | of a recorded cell that are positive in |
| | logarithmic scale base 10. |
| Speed change | The sum of the instantaneous speeds in |
| | logarithmic scale base 10 divided by the |
| | time that a recorded cell was recorded |
| | (numbers of frames where it is detected). |
| Fluctuation | The number of times that the speeds in |
| | logarithmic scale base 10 change from |
| | positive to negative divided by the time |
| | that a recorded cell was recorded (numbers |
| | of frames where it is detected). |

### Example 23. Use of Machine Learning to Distinguish Infectious SIRS Patients from Non-Infectious SIRS Patients

The present example is directed to the use of machine learning (ML) to determine whether a systemic inflammatory response syndrome (SIRS) in a patient is due to infectious or non-infectious causes. Blood samples from infectious and non-infectious SIRS patients were collected and used for leukocyte adhesive function assay (LAFA) using VCAM-1 as substrate, as described in Example 7 and Example 20. The data from infectious and non-infectious blood samples were used to train the machine learning algorithm, and the ability of the trained algorithm to predict unknown samples was then determined.

ML is a computer science field that evolved from artificial intelligence and pattern recognition. ML algorithms enable computers to learn and make predictions from data without human input.

The present machine learning algorithm is based on the Random Forest ensemble learning method. This algorithm was trained using a number of LAFA markers (as described in Example 22) from infectious and non-infectious SIRS patient blood samples. Random Forest constructs a multitude of decision trees, each being trained on a different subset of the training data set. By averaging multiple independent decision trees, Random Forest lowers the risk of overfitting and thus increases the performance of the final model.

The current Random Forest algorithm was trained using data from three infectious SIRS blood samples and three non-infectious SIRS blood samples, all of which were determined by two ICU specialists based on patients' clinical records and blood culture results (Example 7). After this, the ability of the trained algorithm to distinguish infectious SIRS from non-infectious SIRS was tested.

The LAFA data from two infectious and two non-infectious SIRS patient blood samples were used as “unknown” samples to test the trained algorithm. The pathological causes (infectious or non-infectious) of all four samples used for testing were pre-determined by the ICU specialists (Example 7). The algorithm successfully predicted the pathological causes of all four blood samples, providing a 100% accuracy. With an increased number of patient samples for training, the most discriminating LAFA markers that are required to maintain the accuracy of the ML algorithm may be identified. Thus, the existing ML algorithm may be optimised and constructed by using the most useful LAFA markers.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

All publications discussed and/or referenced herein are incorporated herein in their entirety.

The present application claims priority to AU 2018901305, the entire contents of which are incorporated herein in their entirety.

Any discussion of documents, acts, materials, devices, articles, or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### REFERENCES

Beltman and de Boer (2009). Nature reviews. Immunology, 9: 789-798.
Ibbotson et al. (2001) Nature Medicine, 7(4):465-470
Kim and. Herr (2013) Biomicrofluidics, 7: 41501
Kucik et al. (1996) The Journal of clinical investigation, 97: 2139-2144
Sun et al. (2012) Solid-State Device Research Conferernce (ESSDERC) DOI:10.1109/ESSDERC.2012.6343324
Thomas et al. (2002) Clin Immunol, 105(3): 259-272
Turtle and Riddell (2010) Cancer J, 16: 374-381
Vaidyanatahn et al. (2014) Anal Chem, 86(4):2042-2049.

1. A method to discriminate between an infectious and non-infectious inflammatory immune response in a subject, the method comprising:
subjecting a blood sample from the subject to at least one leukocyte function assay (LAFA), wherein the LAFA assesses leukocyte recruitment, adhesion and/or migration to at least one endothelial cell molecule; and
based at least in part on one or more results of the at least one LAFA, determine whether the subject has an infectious inflammatory immune response or a non-infectious inflammatory immune response.

2. The method of claim 1, wherein the at least one LAFA quantitatively and/or semi-quantitatively assesses leukocyte recruitment, adhesion and/or migration.

3. (canceled)

4. The method of claim 1, wherein the at least one endothelial cell molecule is selected from VCAM-1, MadCAM-1, IL-8, SDF-1α, E-Selectin, P-Selectin and ICAM-1.

5. (canceled)

6. The method of claim 1, wherein the at least one LAFA measures one or more of the following parameters: a quantification of rolling leukocyte cells detected, a quantification of adhesion leukocyte cells detected, a quantification of crawling cells detected, an average speed of individual leukocyte cells detected, an average straightness of individual leukocyte cells detected, an average displacement of individual leukocyte cells detected and an average dwell time of individual cells detected.

7. The method of claim 1, wherein the results of the at least one LAFA from the blood sample from the subject is used as a reference level for generating one or more parameters that are used for generating one or more indexes.

8. The method of claim 8, wherein the results of the at least one LAFA from at least one healthy blood sample is used as a reference level for generating one or more parameters that are used of generating one or more indexes.

9. The method of claim 1, wherein an activation potential ratio of the subject's blood is generated based on the results of at least one LAFA from the blood of the subject divided by the results of at least one LAFA from a Mn2+ treated blood sample of the subject.

10. The method of claim 1, wherein the method further comprises detecting one or more leukocyte cell surface markers.

11.-17. (canceled)

18. The method of claim 1, wherein a LAFA result comprising:
i) a higher or lower level of recruited and/or adhesive leukocytes;
ii) a higher or lower percentage of recruited and/or adhesive neutrophils; and/or
iii) a higher or lower level of recruited and/or adhesive monocytes,
as compared to the reference level is indicative of sepsis, wherein the reference level is derived from a population of subjects known to have non-infectious SIRS.

19. The method of claim 1, wherein the method comprises determining that the subject has an infectious inflammatory immune response and administering an antimicrobial or antiviral composition to the subject.

20. The method of claim 1, wherein the method comprises determining that the subject has a non-infectious inflammatory immune response and administering an anti-inflammatory composition to the subject.

21. method of claim 1, wherein the method comprises determining that the subject has a non-infectious inflammatory response and administering to the subject a drug capable of altering leukocyte recruitment, adhesion and/or migration.

22. The method of claim 21, wherein the drug is an antibody that interferes with the binding of a leukocyte adhesion molecule to an endothelial cell molecule.

23. A method of treating an infectious inflammatory immune response in a subject, the method comprising performing the method according to claim 1 and determining that the subject has an infectious inflammatory immune response, and treating the subject for the infectious inflammatory immune response.

24. The method of claim 23, wherein the subject has sepsis.

25. The method of claim 24, wherein treating the subject for sepsis comprises treating the patient with one or more of an antibiotic, vasopressor and corticosteroid.

26. A method to assess a subject's response, or potential response, to a drug suitable for treating an infectious disease, the method comprising:
subjecting a blood sample from the subject to at least one leukocyte function assay (LAFA), wherein the LAFA assesses leukocyte recruitment, adhesion and/or migration to at least one endothelial cell molecule; and
based at least in part on one or more results of the at least one LAFA, assess a patient's response, or potential response, to the drug for treating the infectious disease.

27. A method of detecting a subset of leukocytes in a subject having an inflammatory immune response, the method comprising subjecting a blood sample from the subject to at least one leukocyte function assay (LAFA), wherein the LAFA assesses leukocyte recruitment, adhesion and/or migration to at least one endothelial cell molecule;
detecting one or more leukocyte cell surface markers, and
based at least in part on one or more results of the at least one LAFA and detection of one or more leukocyte cell surface markers, determining a subset of leukocytes associated with the inflammatory immune response.

28. The method of claim 27, wherein the subject has an inflammatory condition or infectious disease.

29. The method of claim 27, wherein the subject has, or is suspected of having, SIRS or sepsis.

30.-50. (canceled)
